(19) United States
(12) Patent Application Publication
(10) Pub. No.: US 2022/0389422 A1
(43) Pub. Date: Dec. 8, 2022

(54) IONIZABLE CATIONIC LIPIDS FOR RNA DELIVERY

(71) Applicant: Arcturus Therapeutics, Inc., San Diego, CA (US)

(72) Inventors: Kumar RAJAPPAN, San Diego, CA (US); Steven TANIS, San Diego, CA (US); Amit SAGI, San Diego, CA (US); Priya Prakash KARMALI, San Diego, CA (US)

(21) Appl. No.: 17/737,690

(22) Filed: May 5, 2022

Related U.S. Application Data
(60) Provisional application No. 63/185,303, filed on May 6, 2021.

(51) Int. Cl. C12N 15/113; C07C 327/34; C07C 229/24; A61K 9/127; A61K 9/19; A61K 9/51; A61K 47/14; A61K 9/00; A61P 37/02

(52) U.S. Cl. CPC C12N 15/113; C07C 327/34; C07C 229/24; A61K 9/1272; A61K 9/19; A61K 9/5123; A61K 9/5146; A61K 47/14; A61K 9/0021; A61P 37/02

(57) ABSTRACT

The present disclosure describes compounds of Formula (I) or a pharmaceutically acceptable salt thereof:

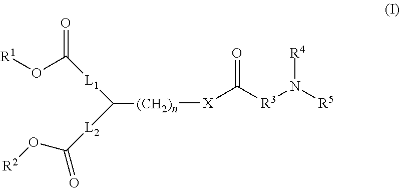

wherein: R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11; L¹ and L² are each independently absent, a linear C₁₋₅ alkylene, or (CH₂)ₚ—O—(CH₂)_{q}, wherein p and q are each independently 1-3; R³ is a linear C₂₋₅ alkylene optionally substituted with one or two methyl groups; R⁴ and R⁵ are each independently H or C₁₋₆ alkyl; X is O or S; and n is 0-2.

### CROSS-REFERENCE TO RELATED APPLICATIONS

This application claims priority to U.S. Provisional Application No. 63/185,303, filed May 6, 2021, which is hereby incorporated by reference in its entirety.

### TECHNICAL FIELD

Embodiments herein relate generally to lipids. In particular, embodiments herein relate to new lipids and lipid compositions that facilitate the intracellular delivery of biologically active and therapeutic molecules.

### BACKGROUND

The variety of nucleic acid-based therapeutics for targeted delivery creates a challenge for lipid-based delivery vehicles. For example, nucleic acids are structurally diverse in size and type. Examples include DNA used in gene therapy, plasmids, small interfering nucleic acids (siNA), and microRNA (miRNA) for use in RNA interference (RNAi), antisense molecules, ribozymes, antagomirs, and aptamers.

The design and use of cationic lipids and ionizable cationic lipids for inclusion in such lipid-based delivery vehicles has shown great advantages. However, use of these lipids can contribute to significant side effects when administered in vivo. One problem that has been observed includes low biodegrability and clearance from target tissues, thus creating an in vivo build up of the lipid. Another problem is that large amounts of the lipid may cause an adverse immunogenic effect, which can result in discomfort in the subject and a decrease in the therapeutics effect of the active ingredient. A third problem associated with many cationic lipids is a low percentage of effective delivery to the target, thus resulting in a relatively low therapeutic effect or low potency. Finally, it is important that the cationic lipid in the delivery vehicle have a specially tuned pH so it can formulate with the active and protect it from degradation during administration, but be able to release the active once the vehicle has reached its target. Thus, there is a need in the art for the development of new lipids that can meet the special needs of lipid-nucleic acid delivery systems.

### SUMMARY

The present disclosure provides lipids of Formula (I) as described herein useful for lipid-based delivery of nucleic acids and other therapeutic agents for treating diseases. These and other uses will be apparent to those skilled in the art. Additional features and advantages of the subject technology will be set forth in the description below, and in part will be apparent from the description, or may be learned by practice of the subject technology. The advantages of the subject technology will be realized and attained by the structures particularly pointed out in the written description and embodiments hereof as well as the appended drawings.

It is to be understood that both the foregoing general description and the following detailed description are exemplary and explanatory and are intended to provide further explanation of the subject technology.

In some embodiments, the present disclosure provides a compound of Formula I, or a pharmaceutically acceptable salt thereof:

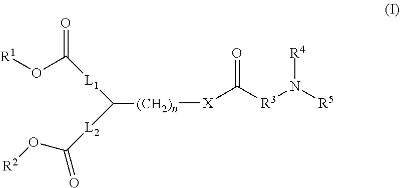

wherein: R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11; L¹ and L² are each independently absent, a linear C₁₋₅ alkylene, or (CH₂)ₚ—O—(CH₂)_{q}, wherein p and q are each independently 1-3; R³ is a linear C₂₋₅ alkylene optionally substituted with one or two methyl groups; R⁴ and R⁵ are each independently H or C₁₋₆ alkyl; X is O or S; and n is 0-2.

In some embodiments, the present disclosure provides a lipid nanoparticle, comprising a plurality of ligands, wherein each ligand is independently a compound described herein, wherein the plurality of ligands self-assembles to form the lipid nanoparticle comprising an interior and exterior.

In some embodiments, the present disclosure provides a pharmaceutical composition comprising the compound described herein or the lipid nanoparticle described herein, and a pharmaceutically acceptable excipient.

In some embodiments, the present disclosure provides a method of treating a disease in a subject in need thereof, comprising administering a therapeutically effective amount to the subject the compound described herein, the lipid nanoparticle described herein, or the pharmaceutical composition described herein.

In some embodiments, the present disclosure provides a method of delivering a nucleic acid to a subject in needed thereof, comprising encapsulating a therapeutically effective amount of the a nucleic acid in the the lipid nanoparticle described herein, and administering the lipid nanoparticle to the subject.

### DETAILED DESCRIPTION

### I. General

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

### II. Definitions

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

The term “lower alkyl” means a group having one to six carbons in the chain which chain may be straight or branched. Non-limiting examples of suitable alkyl groups include methyl, ethyl, n-propyl, isopropyl, n-butyl, t-butyl, n-pentyl, and hexyl.

The term “amino,” as used herein, represents N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkylcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkylheterocyclyl (e.g., alkylheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the disclosure can be an unsubstituted amino (i.e., —NH₂) or a substituted amino (i.e., —N(R′)₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₁₋₁₀ aryl.

The term “anionic lipid” means a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The terms “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The term “cationic lipid” means amphiphilic lipids and salts thereof having a positive, hydrophilic head group; one, two, three, or more hydrophobic fatty acid or fatty alkyl chains; and a connector between these two domains. An ionizable or protonatable cationic lipid is typically protonated (i.e., positively charged) at a pH below its pKa and is substantially neutral at a pH above the pKa. Preferred ionizable cationic lipids are those having a pKa that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, 7-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA)(also known as 1-Bl 1).

The term “comprising” is intended to be open and permits but does not require the inclusion of additional elements or steps. When the term “comprising” is used herein, the term “consisting of” is thus also encompassed and disclosed.

The term “in combination with” means the administration of a lipid formulated mRNA of the present disclosure with other medicaments in the methods of treatment of this disclosure, means-that the lipid formulated mRNA of the present disclosure and the other medicaments are administered sequentially or concurrently in separate dosage forms, or are administered concurrently in the same dosage form.

The term “commercially available chemicals” and the chemicals used in the Examples set forth herein may be obtained from standard commercial sources, where such sources include, for example, Acros Organics (Pittsburgh, Pa.), Sigma-Adrich Chemical (Milwaukee, Wis.), Avocado Research (Lancashire, U.K.), Bionet (Cornwall, U.K.), Boron Molecular (Research Triangle Park, N.C.), Combi-Blocks (San Diego, Calif.), Eastman Organic Chemicals, Eastman Kodak Company (Rochester, N.Y.), Fisher Scientific Co. (Pittsburgh, Pa.), Frontier Scientific (Logan, Utah), ICN Biomedicals, Inc. (Costa Mesa, Calif.), Lancaster Synthesis (Windham, N.H.), Maybridge Chemical Co. (Cornwall, U.K.), Pierce Chemical Co. (Rockford, Ill.), Riedel de Haen (Hannover, Germany), Spectrum Quality Product, Inc. (New Brunswick, N.J.), TCI America (Portland, Or.), and Wako Chemicals USA, Inc. (Richmond, Va.).

The phrase “compounds described in the chemical literature” may be identified through reference books and databases directed to chemical compounds and chemical reactions, as known to one of ordinary skill in the art. Suitable reference books and treatise that detail the synthesis of reactants useful in the preparation of compounds disclosed herein, or provide references to articles that describe the preparation of compounds disclosed herein, include for example, “Synthetic Organic Chemistry”, John Wiley and Sons, Inc. New York; S. R. Sandler et al, “Organic Functional Group Preparations,” 2nd Ed., Academic Press, New York, 1983; H. O. House, “Modern Synthetic Reactions,” 2nd Ed., W. A. Benjamin, Inc. Menlo Park, Calif., 1972; T. L. Glichrist, “Heterocyclic Chemistry,” 2nd Ed. John Wiley and Sons, New York, 1992; J. March, “Advanced Organic Chemistry: reactions, Mechanisms and Structure,” 5th Ed., Wiley Interscience, New York, 2001; Specific and analogous reactants may also be identified through the indices of known chemicals prepared by the Chemical Abstract Service of the American Chemical Society, which are available in most public and university libraries, as well as through online databases (the American Chemical Society, Washington, D.C. may be contacted for more details). Chemicals that are known but not commercially available in catalogs may be prepared by custom chemical synthesis houses, where many of the standard chemical supply houses (such as those listed above) provide custom synthesis services.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “fully encapsulated” means that the nucleic acid (e.g., mRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

The term “compound,” is meant to include all stereoisomers, geometric isomers, tautomers, and isotopes of the structures depicted.

The term “delivery” refers to the act or manner of delivering a compound, substance, entity, moiety, cargo or payload.

The term “feature” refers to a characteristic, a property, or a distinctive element.

The term “fragment,” as used herein, refers to a portion. For example, fragments of proteins may comprise polypeptides obtained by digesting full-length protein isolated from cultured cells.

The term “hydrophobic lipids” means compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

The term “lipid delivery vehicle” means a lipid formulation that can be used to deliver a therapeutic nucleic acid (e.g., mRNA) to a target site of interest (e.g., cell, tissue, organ, and the like). The lipid delivery vehicle can be a nucleic acid-lipid particle, which can be formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

The term “lipid encapsulated” means a lipid particle that provides a therapeutic nucleic acid such as an mRNA with full encapsulation, partial encapsulation, or both. In a preferred embodiment, the nucleic acid (e.g., mRNA) is fully encapsulated in the lipid particle.

The term “amphipathic lipid” or “amphiphilic lipid” means the material in which the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

The term “linker” or “linking moiety” refers to a group of atoms, e.g., 10-100 atoms, and can be comprised of the atoms or groups such as, but not limited to, carbon, amino, alkylamino, oxygen, sulfur, sulfoxide, sulfonyl, carbonyl, and imine. The linker may be of sufficient length as to not interfere with incorporation into an amino acid sequence. Examples of chemical groups that can be incorporated into the linker include, but are not limited to, alkyl, alkenyl, alkynyl, amido, amino, ether, thioether, ester, alkyl, heteroalkyl, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond, which can be cleaved for example by the use of tris(2-carboxyethyl)phosphine (TCEP), or other reducing agents, and/or photolysis, as well as an ester bond, which can be cleaved for example by acidic or basic hydrolysis.

The term “mammal” means a human or other mammal or means a human being.

The term “messenger RNA” (mRNA) refers to any polynucleotide which encodes a protein or polypeptide of interest and which is capable of being translated to produce the encoded protein or polypeptide of interest in vitro, in vivo, in situ or ex vivo.

The term “modified” refers to a changed state or structure of a molecule of the disclosure. Molecules may be modified in many ways including chemically, structurally, and functionally. In one embodiment, nucleic acid active ingredients are modified by the introduction of non-natural nucleosides and/or nucleotides, e.g., as it relates to the natural ribonucleotides A, U, G, and C. Noncanonical nucleotides such as the cap structures are not considered “modified” although they may differ from the chemical structure of the A, C, G, U ribonucleotides.

The term “naturally occurring” means existing in nature without artificial aid.

The term “nonhuman vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

The term “patient” refers to a subject who may seek or be in need of treatment, requires treatment, is receiving treatment, will receive treatment, or a subject who is under care by a trained professional for a particular disease or condition.

The phrase “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “physicochemical” means of or relating to a physical and/or chemical property.

The term “phosphate” is used in its ordinary sense as understood by those skilled in the art and includes its protonated forms, for example

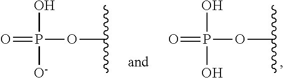 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

The term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

The terms “significant” or “significantly” are used synonymously with the term “substantially.”

The phrase “single unit dose” is a dose of any therapeutic administered in one dose/at one time/single route/single point of contact, i.e., single administration event.

The term “siRNA” or small interfering RNA, sometimes known as short interfering RNA or silencing RNA, refers to a class of double-stranded RNA non-coding RNA molecules, typically 18-27 base pairs in length, similar to miRNA, and operating within the RNA interference (RNAi) pathway. It interferes with the expression of specific genes with complementary nucleotide sequences by degrading mRNA after transcription, thereby preventing translation.

The term “solvate” means a physical association of a compound of this disclosure with one or more solvent molecules. This physical association involves varying degrees of ionic bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The term “split dose” is the division of single unit dose or total daily dose into two or more doses.

The term “stable” refers to a compound that is sufficiently robust to survive isolation to a useful degree of purity from a reaction mixture, and preferably capable of formulation into an efficacious therapeutic agent.

The terms “stabilize”, “stabilized,” “stabilized region” means to make or become stable.

The term “substituted” means substitution with specified groups other than hydrogen, or with one or more groups, moieties, or radicals which can be the same or different, with each, for example, being independently selected.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The phrase “Substantially equal” relates to time differences between doses, the term means plus/minus 2%.

The phrase “substantially simultaneously” relates to plurality of doses, the term means within 2 seconds.

The phrase “suffering from” relates to an individual who is “suffering from” a disease, disorder, and/or condition has been diagnosed with or displays one or more symptoms of a disease, disorder, and/or condition.

The phrase “susceptible to” relates to an individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or polypeptides or other molecules of the present disclosure may be chemical or enzymatic.

The term “therapeutic agent” refers to any agent that, when administered to a subject, has a therapeutic, diagnostic, and/or prophylactic effect and/or elicits a desired biological and/or pharmacological effect.

The term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “total daily dose” is an amount given or prescribed in 24 hour period. It may be administered as a single unit dose.

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by enantio selective and/or stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The term “half-life” is the time required for a quantity such as nucleic acid or protein concentration or activity to fall to half of its value as measured at the beginning of a time period.

The term “in vitro” refers to events that occur in an artificial environment, e.g., in a test tube or reaction vessel, in cell culture, in a Petri dish, etc., rather than within an organism (e.g., animal, plant, or microbe).

The term “in vivo” refers to events that occur within an organism (e.g., animal, plant, or microbe or cell or tissue thereof).

The term “monomer” refers to a single unit, e.g., a single nucleic acid, which may be joined with another molecule of the same or different type to form an oligomer. In some embodiments, a monomer may be an unlocked nucleic acid, i.e., a UNA monomer.

The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” means an amphipathic lipid or a neutral lipid or anionic lipid and is described herein.

The terms “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “translatable” may be used interchangeably with the term “expressible” and refers to the ability of polynucleotide, or a portion thereof, to be converted to a polypeptide by a host cell. As is understood in the art, translation is the process in which ribosomes in a cell's cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications, and equivalents. In particular, this disclosure includes any combination of the features, terms, or elements of the various illustrative components and examples.

II. COMPOUNDS

In some embodiments, the present disclosure provides a compound of Formula I, or a pharmaceutically acceptable salt thereof:

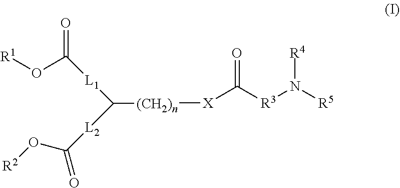

wherein: R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11; L¹ and L² are each independently absent, a linear C₁₋₅ alkylene, or (CH₂)ₚ—O—(CH₂)_{q}, wherein p and q are each independently 1-3; R³ is a linear C₂₋₅ alkylene optionally substituted with one or two methyl groups; R⁴ and R⁵ are each independently H or C₁₋₆ alkyl; X is O or S; and n is 0-2.

In some embodiments, R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—. In some embodiments, R¹ and R² are each independently (CH₃(CH₂)ₘ)₂CH—, (CH₃(CH₂)ₘ)(CH₃(CH₂)ₘ₋₁)CH, (CH₃(CH₂)ₘ)₂CHCH₂—, or (CH₃(CH₂)ₘ)(CH₃(CH₂)ₘ₋₁)CHCH₂—. In some embodiments, R¹ and R² are each independently selected from (CH₃(CH₂)ₘ)₂CH—, and (CH₃(CH₂)ₘ)₂CHCH₂—. In some embodiments, R¹ and R² are each independently (CH₃(CH₂)ₘ)₂CH—. In some embodiments, R¹ and R² are each independently (CH₃(CH₂)ₘ)₂CHCH₂—. In some embodiments, R¹ and R² are each independently selected from (CH₃(CH₂)ₘ)(CH₃(CH₂)ₘ₋₁)CH, (CH₃(CH₂)ₘ)(CH₃(CH₂)ₘ₋₂)CH, and (CH₃(CH₂)ₘ)(CH₃(CH₂)ₘ₋₁)CHCH₂—. In some embodiments, R¹ is (CH₃(CH₂)ₘ)₂CH— or (CH₃(CH₂)ₘ)₂CHCH₂— and R² is selected from (CH₃(CH₂)ₘ)(CH₃(CH₂)ₘ₋₁)CH, (CH₃(CH₂)ₘ)(CH₃(CH₂)ₘ₋₂)CH, and (CH₃(CH₂)ₘ)(CH₃(CH₂)ₘ₋₁)CHCH₂—.

In some embodiments, m is 4 to 11. In some embodiments, m is 4 to 9. In some embodiments, m is 4 to 8. In some embodiments, m is 5 to 7. In some embodiments, m is 5. In some embodiments, m is 6. In some embodiments, m is 7.

In some embodiments, L¹ and L² are each independently absent, a linear C₁₋₅ alkylene, or (CH₂)ₚ—O—(CH₂)_{q}. In some embodiments, L¹ and L² are each independently C₁₋₅ alkylene or (CH₂)ₚ—O—(CH₂)_{q}. In some embodiments, L¹ and L² are each independently C₂₋₅ alkylene or (CH₂)ₚ—O—(CH₂)_{q}. In some embodiments, L¹ and L² are each independently C₂₋₅ alkylene. In some embodiments, L¹ and L² are each propylene. In some embodiments, L¹ and L² are each independently C₂₋₅ alkylene. In some embodiments, L¹ and L² are each independently (CH₂)ₚ—O—(CH₂)_{q}. In some embodiments, L¹ and L² are each independently absent.

In some embodiments, p and q are each independently 1-3. In some embodiments, p and q are each independently 1-2. In some embodiments, p and q are each independently 1. In some embodiments, p and q are each independently 2. In some embodiments, p and q are each independently 3.

In some embodiments, R³ is a linear C₂₋₅ alkylene optionally substituted with one or two methyl groups. In some embodiments, R³ is a linear C₂₋₅ alkylene. In some embodiments, R³ is C₃₋₅ alkylene. In some embodiments, R³ is C₁₋₃ alkylene. In some embodiments, R³ is propylene.

In some embodiments, R⁴ and R⁵ are each independently H or C₁₋₆ alkyl. In some embodiments, R⁴ and R⁵ are each independently C₁₋₆ alkyl. In some embodiments, R⁴ and R⁵ are each independently C1.3 alkyl. In some embodiments, R⁴ and R⁵ are each independently methyl. In some embodiments, R⁴ and R⁵ are each independently H.

In some embodiments, X is O or S. In some embodiments, X is O. In some embodiments, X is S.

In some embodiments, n is 0-2. In some embodiments, n is 0-1. In some embodiments, n is 0. In some embodiments, n is 1. In some embodiments, n is 2.

In some embodiments, the compound is selected from the group consisting of:

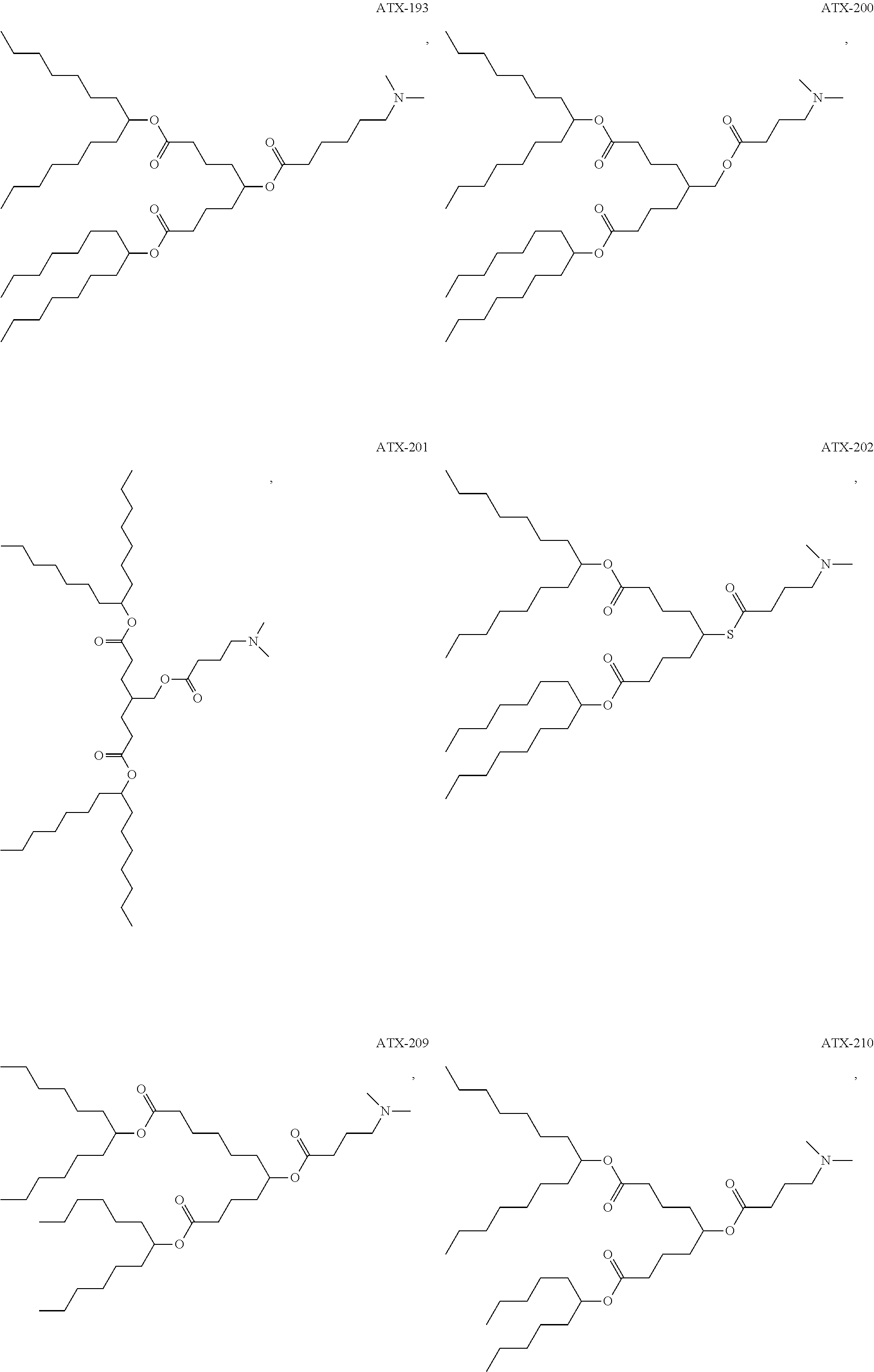 [SMILES: CCCCCCCC(CCCCCCC)OC(=O)CCCC(CCCC(=O)OC(CCCCCCC)CCCCCCC)OC(=O)CCCCCN(C)C ; CCCCCCCC(CCCCCCC)OC(=O)CCCC(CCCC(=O)OC(CCCCCCC)CCCCCCC)COC(=O)CCCN(C)C ; CCCCCCCC(CCCCCCC)OC(=O)CCC(CCC(=O)OC(CCCCCCC)CCCCCCC)COC(=O)CCCN(C)C ; CCCCCCCC(CCCCCCC)OC(=O)CCCC(CCCC(=O)OC(CCCCCCC)CCCCCCC)SC(=O)CCCN(C)C ; CCCCCCC(CCCCCC)OC(=O)CCCCCC(CCCC(=O)OC(CCCCCC)CCCCCC)OC(=O)CCCN(C)C ; CCCCCCCC(CCCCCCC)OC(=O)CCCC(CCCC(=O)OC(CCCCC)CCCCC)OC(=O)CCCN(C)C]

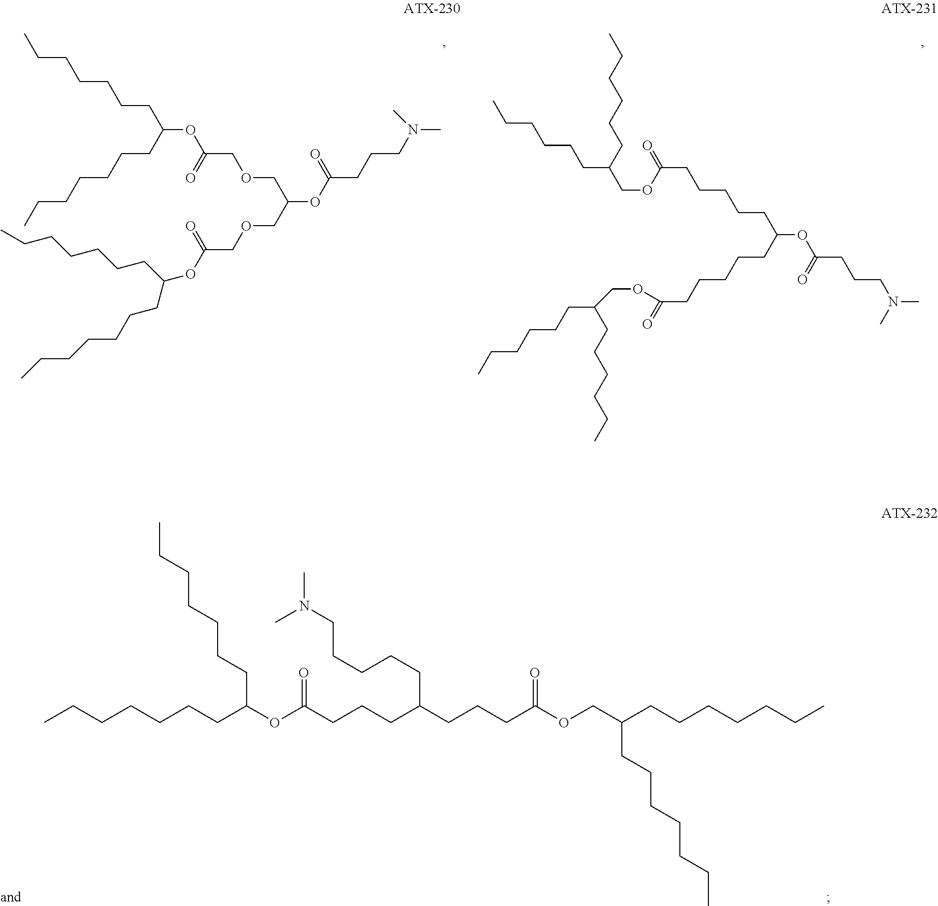 [SMILES: CCCCCCCC(CCCCCCC)OC(=O)COCC(COCC(=O)OC(CCCCCCC)CCCCCCC)OC(=O)CCCN(C)C ; CCCCCCC(CCCCCC)COC(=O)CCCCCC(CCCCCC(=O)OCC(CCCCCC)CCCCCC)OC(=O)CCCN(C)C ; CCCCCCCC(CCCCCCC)COC(=O)CCCC(CCCCCN(C)C)CCCC(=O)OC(CCCCCCC)CCCCCCC]

or a pharmaceutically acceptable salt thereof.

In some embodiments, the compound is ATX-193. In some embodiments, the compound is ATX-200. In some embodiments, the compound is ATX-201. In some embodiments, the compound is ATX-202. In some embodiments, the compound is ATX-209. In some embodiments, the compound is ATX-210. In some embodiments, the compound is ATX-230. In some embodiments, the compound is ATX-231. In some embodiments, the compound is ATX-232.

In some embodiments, the present invention provides a lipid composition comprising a nucleic acid and a compound of the present invention. In some embodiments, the nucleic acid is selected from an siRNA, an mRNA, a self-replicating RNA, a DNA plasmid, and an antisense oligonucleotide. In some embodiments, the nucleic acid is a mRNA or a self-replicating RNA comprising a coding region that encodes a therapeutic protein of interest. In some embodiments, the therapeutic protein of interest is an enzyme, and antibody, an antigen, a receptor, or a transporter. In some embodiments, the therapeutic protein of interest is a gene-editing enzyme. In some embodiments, the gene-editing enzyme is selected from a TALEN, a CRISPR, a meganuclease, or a zinc finger nuclease. In some embodiments, the lipid composition comprises liposomes, lipoplexes, or lipid nanoparticles.

### IV. Lipid Formulations and Nanoparticles

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), and release at the cytoplasm (for RNA). Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still undergoing development. The use of mRNA in lipid delivery vehicles quickly rose to prominence as a result of the COVID-19 pandemic with several vaccines delivering mRNA encoding the spike protein of COVID-19 showing strong protective capabilities. Such lipid-based mRNA vaccines include Pfizer and BioNtech's BNT162b2 and Moderna's mRNA-1273, which have received emergency use authorization around the world.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and are within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicles (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int. J. Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid is contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed hereinbelow.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell can be formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation.

In some embodiments, the lipid nanoparticle comprises a lipid of Formula I.

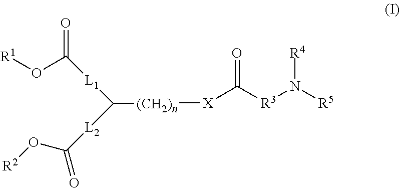

or a pharmaceutically acceptable salt or solvate thereof, wherein: R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11; L¹ and L² are each independently absent, a linear C₁₋₅ alkylene, or (CH₂)ₚ—O—(CH₂)_{q}, wherein p and q are each independently 1-3; R³ is a linear C₂₋₅ alkylene optionally substituted with one or two methyl groups; R⁴ and R⁵ are each independently H or C1.6 alkyl; X is O or S; and n is 0-2.

In some embodiments, any one or more lipids recited herein may be expressly excluded.

In some embodiments, the present disclosure provides a lipid nanoparticle, comprising a plurality of ligands, wherein each ligand is independently a compound described herein, wherein the plurality of ligands self-assembles to form the lipid nanoparticle comprising an interior and exterior.

In some embodiments, the average size of the lipid nanoparticle is about 100 nm. In some embodiments, the average size of the lipid nanoparticle is less than about 100 nm. In some embodiments, the average particle size of the lipid nanoparticle is about 40 nm to about 100 nm. In some embodiments, the average particle size of the lipid nanoparticle is about 50 nm to about 90 nm. In some embodiments, the average particle size of the lipid nanoparticle is about 55 nm to about 85 nm.

In some embodiments, the lipid nanoparticle further comprises nucleic acids in the interior. In some embodiments, the nucleic acid is selected from an siRNA, an mRNA, a self-replicating RNA, a DNA plasmid, and an antisense oligonucleotide. In some embodiments, the nucleic acid is a mRNA or a self-replicating RNA comprising a coding region that encodes a therapeutic protein of interest. In some embodiments, the therapeutic protein of interest is an enzyme, and antibody, an antigen, a receptor, or a transporter. In some embodiments, the therapeutic protein of interest is a gene-editing enzyme. In some embodiments, the gene-editing enzyme is selected from a TALEN, a CRISPR, a meganuclease, or a zinc finger nuclease.

In some embodiments, the lipid nanoparticle further comprises siRNA or mRNA in the interior. In some embodiments, the the lipid nanoparticle further comprises mRNA in the interior.

In some embodiments, the lipid nanoparticle further comprises a helper lipid as described below. In some embodiments, the lipid nanoparticle further comprises PEG-lipid conjugates as described herein.

In some embodiments, the lipid nanoparticle comprises about 45 mol % to 65 mol % of the compound of the present invention, about 2 mol % to about 15 mol % of a helper lipid, about 20 mol % to about 42 mol % of cholesterol, and about 0.5 mol % to about 3 mol % of a PEG-lipid conjugate. In some embodiments, the lipid nanoparticle comprises about 50 mol % to about 61 mol % of the compound of the present invention, about 5 mol % to about 9 mol % of the helper lipid, about 29 mol % to about 38 mol % of cholesterol, and about 1 mol % to about 2 mol % of the PEG-lipid conjugate. In some embodiments, the lipid nanoparticle comprises about 56 mol % to about 58 mol % of the compound of the present invention, about 6 mol % to about 8 mol % of DSPC, about 31 mol % to about 34 mol % of cholesterol, and about 1.25 mol % to about 1.75 mol % of the PEG-lipid conjugate.

In some embodiments, the lipid nanoparticle comprises about 50 mol % to 61 mol % of the compound of the present invention, about 2 mol % to about 12 mol % of DSPC, about 25 mol % to about 42 mol % of cholesterol, and about 0.5 mol % to a bout 3 mol % of PEG2000-DMG. In some embodiments, the lipid nanoparticle comprises about 50 mol % to about 61 mol % of the compound of the present invention, about 5 mol % to about 9 mol % of DSPC, about 29 mol % to about 38 mol % of cholesterol, and about 1 mol % to about 2 mol % of PEG2000-DMG. In some embodiments, the lipid nanoparticle comprises about 56 mol % to about 58 mol % of the compound of the present invention, about 6 mol % to about 8 mol % of DSPC, about 31 mol % to about 34 mol % of cholesterol, and about 1.25 mol % to about 1.75 mol % of PEG2000-DMG.

In some embodiments, the lipid nanoparticle has a total lipid:nucleic acid weight ratio of about 50:1 to about 10:1. In some embodiments, the lipid nanoparticle has a total lipid:nucleic acid weight ratio of about 40:1 to about 20:1. In some embodiments, the lipid nanoparticle has a total lipid:nucleic acid weight ratio of about 35:1 to about 25:1. In some embodiments, the lipid nanoparticle has a total lipid:nucleic acid weight ratio of about 32:1 to about 28:1. In some embodiments, the lipid nanoparticle has a total lipid:nucleic acid weight ratio of about 31:1 to about 29:1.

In some embodiments, the lipid nanoparticle has a total lipid:mRNA weight ratio of about 50:1 to about 10:1. In some embodiments, the lipid nanoparticle has a total lipid:mRNA weight ratio of about 40:1 to about 20:1. In some embodiments, the lipid nanoparticle has a total lipid:mRNA weight ratio of about 35:1 to about 25:1. In some embodiments, the lipid nanoparticle has a total lipid:mRNA weight ratio of about 32:1 to about 28:1. In some embodiments, the lipid nanoparticle has a total lipid:mRNA weight ratio of about 31:1 to about 29:1.

In some embodiments, the lipid nanoparticle nanoparticle comprises a HEPES buffer at a pH of about 7.4. In some embodiments, the HEPES buffer is at a concentration of about 7 mg/mL to about 15 mg/mL. In some embodiments, the lipid nanoparticle further comprises about 2.0 mg/mL to about 4.0 mg/mL of NaCl.

In some embodiments, the lipid nanoparticle further comprises one or more cryoprotectants. In some embodiments, the one or more cryoprotectants are selected from sucrose, glycerol, or a combination of sucrose and glycerol. In some embodiments, the lipid nanoparticle comprises a combination of sucrose at a concentration of about 70 mg/mL to about 110 mg/mL and glycerol at a concentration of about 50 mg/mL to about 70 mg/mL.

### Lipid-Nucleic Acid Formulations

A nucleic acid or a pharmaceutically acceptable salt thereof can be incorporated into a lipid formulation (i.e., a lipid-based delivery vehicle).

In the context of the present disclosure, a lipid-based delivery vehicle typically serves to transport a desired nucleic acid (siRNA, plasmid DNA, mRNA, self-replicating RNA, etc.) to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing a nucleic acid. In some embodiments, the lipid-based delivery vehicle comprises a nanoparticle or a bilayer of lipid molecules and a nucleic acid. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic nucleic acid molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the nucleic acid is fully encapsulated within the lipid portion of the lipid formulation such that the nucleic acid in the lipid formulation is resistant in aqueous solution to nuclease degradation. In other embodiments, the lipid formulations described herein are substantially non-toxic to mammals such as humans.

The lipid formulations of the disclosure also typically have a total lipid:nucleic acid ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1. In some preferred embodiments, the total lipid:nucleic acid ratio (mass/mass ratio) is from about 10:1 to about 25:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise a nucleic acid, a cationic lipid (e.g., one or more cationic lipids or salts thereof described herein), a phospholipid, and a conjugated lipid that inhibits aggregation of the particles (e.g., one or more PEG-lipid conjugate and/or other lipid conjugate of the disclosure). The lipid formulations can also include cholesterol.

In some embodiments, the lipid nanoparticle further comprises a PEG-lipid conjugate. In some embodiments, the PEG-lipid conjugate is PEG-DMG. IN some embodiments, the PEG-DMG is PEG2000-DMG.

In the nucleic acid-lipid formulations, the nucleic acid may be fully encapsulated within the lipid portion of the formulation, thereby protecting the nucleic acid from nuclease degradation. In preferred embodiments, a lipid formulation comprising a nucleic acid is fully encapsulated within the lipid portion of the lipid formulation, thereby protecting the nucleic acid from nuclease degradation. In certain instances, the nucleic acid in the lipid formulation is not substantially degraded after exposure of the particle to a nuclease at 37° C. for at least 20, 30, 45, or 60 minutes. In certain other instances, the nucleic acid in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the nucleic acid is complexed with the lipid portion of the formulation.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where I and I0 refer to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of nucleic acid-liposomes. In some embodiments, the nucleic acid-lipid composition comprises a plurality of nucleic acid-cationic liposomes. In some embodiments, the nucleic acid-lipid composition comprises a plurality of nucleic acid-lipid nanoparticles.

In some embodiments, the lipid formulations comprise a nucleic acid that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the nucleic acid encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, expressible polynucleotides, nucleic acid active agents, and mRNA constructs can be lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) a nucleic acid (mRNA, siRNA, etc.),
(b) a lipid of the present disclosure, which may be cationic
(c) optionally a non-cationic lipid (such as a neutral lipid), and
(d) optionally, a sterol.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may include, for example, N,N-dimethyl-N,N-di-9-cis-octadecenylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR, 5s, 6aS)-N,N-dimethyl-2,2-di((9Z, 12Z)-octadeca-9,12-dienyl)tetrahydro-3aH-cyclopenta[d][1,3]dioxol-5-amine, (6Z, 9Z, 28Z, 31Z)-heptatriaconta-6,9,28,31-tetraen-19-yl-4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), 3-((6Z, 9Z, 28Z, 31Z)-heptatriaconta-6,9,28,3 1-tetraen-19-yloxy)-N,N-dimethylpropan-1-amine (MC3 Ether), 4-((6Z, 9Z, 28Z, 31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated alkyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In some embodiments, cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises a lipid of Formula I:

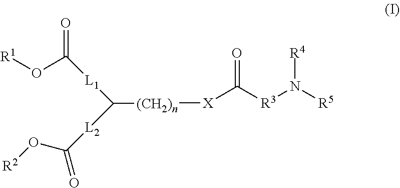

or a pharmaceutically acceptable salt or solvate thereof, wherein: R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11; L¹ and L² are each independently absent, a linear C₁₋₅ alkylene, or (CH₂)ₚ—O—(CH₂)_{q}, wherein p and q are each independently 1-3; R³ is a linear C₂₋₅ alkylene optionally substituted with one or two methyl groups; R⁴ and R⁵ are each independently H or C1.6 alkyl; X is O or S; and n is 0-2.

In some embodiments, any one or more lipids recited herein may be expressly excluded.

### Helper Lipids and Sterols

The mRNA-lipid formulations of the present disclosure can comprise a helper lipid, which can be referred to as a neutral lipid, a neutral helper lipid, non-cationic lipid, non-cationic helper lipid, anionic lipid, anionic helper lipid, or a zwitterionic lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

In some embodiments, the helper lipid is selected from: dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), and phosphatidylcholine (PC). In some embodiments, the helper lipid is distearoylphosphatidylcholine (DSPC).

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g., a cholesterol-free lipid formulation. In yet other embodiments, the helper lipid present in the lipid formulation comprises or consists of cholesterol or a derivative thereof, e.g., a phospholipid-free lipid formulation. In some embodiments, the lipid nanoparticle further comprises cholesterol.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 1 mol % to about 50 mol %, from about 5 mol % to about 48 mol %, from about 5 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation. In some embodiments, the helper lipid comprises from about 1 mol % to about 20 mol %, about 2 mol % to about 12 mol %, about 5 mol % to about 9 mol % or about 6 mol % to about 8 mol %.

In some embodiments, the total of helper lipid in the formulation comprises two or more helper lipids and the total amount of helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation. In some embodiments, the helper lipids are a combination of DSPC and DOTAP. In some embodiments, the helper lipids are a combination of DSPC and DOTMA.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 30 mol % to about 40 mol %, or about 35 mol %, about 36 mol %, about 37 mol %, about 38 mol %, about 39 mol %, or about 40 mol % of the total lipid present in the lipid formulation.

The percentage of helper lipid present in the lipid formulation is a target amount, and the actual amount of helper lipid present in the formulation may vary, for example, by +5 mol %.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a nucleic acid-lipid formulations described herein, the lipid formulation enters cells through receptor mediated endocytosis. Prior to endocytosis, functionalized ligands such as a the lipid conjugate of the disclosure at the surface of the lipid delivery vehicle can be shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA or self-replicating RNA payloads, the cell's own internal translation processes will then translate the RNA into the encoded protein. The encoded protein can further undergo post-translational processing, including transportation to a targeted organelle or location within the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Deterrent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### V. Pharmaceutical Compositions and Delivery Methods

To facilitate nucleic acid activity (e.g., mRNA expression, or knockdown by an ASO or siRNA) in vivo, the lipid formulation delivery vehicles described herein can be combined with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

The lipid formulations and pharmaceutical compositions of the present disclosure may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

The pharmaceutical compositions described herein can be an inhalable composition. Suitable routes of administration include, for example, intratracheal, inhaled, or intranasal. In some embodiments, the administration results in delivery of the nucleic acid to a lung epithelial cell. In some embodiments, the administration shows a selectivity towards lung epithelial cells over other types of lung cells and cells of the airways.

The pharmaceutical compositions disclosed herein can be formulated using one or more excipients to: (1) increase stability; (2) increase cell transfection; (3) permit a sustained or delayed release (e.g., from a depot formulation of the nucleic acid); (4) alter the biodistribution (e.g., target the nucleic acid to specific tissues or cell types); (5) increase the activity of the nucleic acid or a protein expressed therefrom in vivo; and/or (6) alter the release profile of the nucleic acid or an encoded protein in vivo.

Preferably, the lipid formulations may be administered in a local rather than systemic manner. Local delivery can be affected in various ways, depending on the tissue to be targeted. For example, aerosols containing compositions of the present disclosure can be inhaled (for nasal, tracheal, or bronchial delivery).

Pharmaceutical compositions may be administered to any desired tissue. In some embodiments, the nucleic acid delivered by a lipid formulation or composition of the present disclosure is active in the tissue in which the lipid formulation and/or composition was administered. In some embodiments, the nucleic acid is active in a tissue different from the tissue in which the lipid formulation and/or composition was administered. Example tissues in which the nucleic acid may be delivered include, but are not limited to the lung, trachea, and/or nasal passages, muscle, liver, eye, or the central nervous system.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with a primary DNA construct, or mRNA (e.g., for transplantation into a subject), hyaluronidase, nanoparticle mimics and combinations thereof.

Accordingly, the formulations described herein can include one or more excipients, each in an amount that together increases the stability of the nucleic acid in the lipid formulation, increases cell transfection by the nucleic acid (e.g., mRNA or siRNA), increases the expression of an encoded protein, and/or alters the release profile of the encoded protein, or increases knockdown of a target native nucleic acid. Further, a nucleic acid may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

A dosage form of the composition of this disclosure can be solid, which can be reconstituted in a liquid prior to administration. The solid can be administered as a powder. In some embodiments, the pharmaceutical composition comprises a nucleic acid lipid formulation that has been lyophilized.

In a preferred embodiment, the dosage form of the pharmaceutical compositions described herein can be a liquid suspension of nucleic acid-lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about −70° C. In some embodiments, the suspension is diluted with sterile water prior to inhalable administration. In some embodiments, an inhalable administration comprises diluting the suspension with about 1 volume to about 4 volumes of sterile water. In some embodiments, a lyophilized nucleic acid-lipid nanoparticle formulation can be resuspended in a buffer as described herein.

The compositions and methods of the disclosure may be administered to subjects by a variety of mucosal administration modes, including intranasal and/or intrapulmonary. In some aspects of this disclosure, the mucosal tissue layer includes an epithelial cell layer. The epithelial cell can be pulmonary, tracheal, bronchial, alveolar, nasal, and/or buccal. Compositions of this disclosure can be administered using conventional actuators such as mechanical spray devices, as well as pressurized, electrically activated, or other types of actuators.

The compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form, for example, a lyophilized lipid formulation. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the nucleic acid-lipid formulation or suspended in a pharmaceutical solvent, e.g., water, ethanol, or mixtures thereof.

Nasal and pulmonary spray solutions of the present disclosure typically comprise the nucleic acid, optionally formulated with a surface-active agent, such as a nonionic surfactant (e.g., polysorbate-80), and one or more buffers, provided that the inclusion of the surfactant does not disrupt the structure of the lipid formulation. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure provides a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To formulate compositions for pulmonary delivery within the present disclosure, the nucleic acid-lipid formulation can be combined with various pharmaceutically acceptable additives, as well as a base or carrier for dispersion of the nucleic acid-lipid formulation(s). Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The nucleic acid-lipid formulation may be dispersed in a base or vehicle, which may comprise a hydrophilic compound having a capacity to disperse the nucleic acid-lipid formulation and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the nucleic acid-lipid formulation.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the nucleic acid-lipid formulation may be administered in a time release formulation, for example in a composition which includes a slow release polymer. The nucleic acid-lipid formulation can be prepared with carriers that will protect against rapid release, for example a controlled release vehicle such as a polymer, microencapsulated delivery system, or a bioadhesive gel. Prolonged delivery of the nucleic acid-lipid formulation, in various compositions of the disclosure can be brought about by including in the composition agents that delay absorption, for example, aluminum monostearate hydrogels and gelatin.

It has been demonstrated that nucleic acids can be delivered to the lungs by intratracheal administration of a liquid suspension of the nucleic acid composition and inhalation of an aerosol mist produced by a liquid nebulizer or the use of a dry powder apparatus such as that described in U.S. Pat. No. 5,780,014, incorporated herein by reference.

In certain embodiments, the compositions of the disclosure may be formulated such that they may be aerosolized or otherwise delivered as a particulate liquid or solid prior to or upon administration to the subject. Such compositions may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.010 to about 0.5 mg/kg of nucleic acid per dose) to the subject. For example, in certain embodiments, the compositions of the disclosure are administered to a subject using a metered dose inhaler containing a suspension or solution comprising the composition and a suitable propellant. In certain embodiments, the compositions of the disclosure may be formulated as a particulate powder (e.g., respirable dry particles) intended for inhalation. In certain embodiments, compositions of the disclosure formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 m or smaller). In yet other embodiments, the compositions of the disclosure are formulated to include one or more pulmonary surfactants (e.g., lamellar bodies). In some embodiments, the compositions of the disclosure are administered to a subject such that a concentration of at least 0.010 mg/kg, at least 0.015 mg/kg, at least 0.020 mg/kg, at least 0.025 mg/kg, at least 0.030 mg/kg, at least 0.035 mg/kg, at least 0.040 mg/kg, at least 0.045 mg/kg, at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compositions of the disclosure are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg nucleic acid is administered in one or more doses.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

According to the present disclosure, a therapeutically effective dose of the provided composition, when administered regularly, results in an increased nucleic acid activity level in a subject as compared to a baseline activity level before treatment. Typically, the activity level is measured in a biological sample obtained from the subject such as blood, plasma or serum, urine, or solid tissue extracts. The baseline level can be measured immediately before treatment. In some embodiments, administering a pharmaceutical composition described herein results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment. In some embodiments, administering the provided composition results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment for at least about 24 hours, at least about 48 hours, at least about 72 hours, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

In some embodiments, the present disclosure provides a pharmaceutical compostion comprising the compounds described herein, or the lipid nanoparticle described herein, and a pharmaceutically acceptable excipient.

In some embodiments, the present disclosure provides a method of delivering a nucleic acid to a subject in needed thereof, comprising encapsulating a therapeutically effective amount of the a nucleic acid in the the lipid nanoparticle described herein, and administering the lipid nanoparticle to the subject.

In some embodiments, the present disclosure provides a method of delivering mRNA to a subject in needed thereof, comprising encapsulating a therapeutically effective amount of the mRNA in the the lipid nanoparticle described herein, and administering the lipid nanoparticle to the subject.

### VI. Method of Treatment

In some embodiments, the present disclosure provides a method of treating a disease in a subject in need thereof, comprising administering a therapeutically effective amount to the subject the compound described herein, the lipid nanoparticle described herein, or the pharmaceutical composition described herein. In some embodiments, the compound or lipid nanoparticle is administered intravenously or intramuscularly. In some embodiments, the compound or lipid nanoparticle is administered intravenously. In some embodiments, the compound or lipid nanoparticle is administered intramuscularly.

In some embodiments, a method of treating a disease in a subject in need thereof is provided comprising administering to the subject a lipid composition described herein. In some embodiments, the lipid composition is administered intravenously or intramuscularly. In some embodiments, the lipid composition is administered intravenously. In some embodiments, the lipid composition is administered intramuscularly.

In some embodiments, there are provided a methods of treating a disease or disorder in a mammalian subject. A therapeutically effective amount of a composition comprising a lipid, as disclosed herein, specifically a cationic lipid, a nucleic, an amphiphile, a phospholipid, cholesterol, and a PEG-linked cholesterol may be administered to a subject having a disease or disorder associated with expression or overexpression of a gene that can be reduced, decreased, downregulated, or silenced by the composition. The compositions described herein can be used in a methods for treating cancer or inflammatory disease. The disease may be one selected from the group consisting of central nervous system disorders, peripheral nervous system disorders, muscle atrophies, muscle dystrophies, immune disorder, cancer, renal disease, fibrotic disease, genetic abnormality, inflammation, and cardiovascular disorder.

In some embodiments, the present disclosure provides a method of expressing a protein or polypeptide in a target cell, comprising contacting the target cell with a lipid nanoparticle described herein, or the pharmaceutical composition described herein. In some embodiments, the protein or polypeptide is an antigen, and expression of the antigen provides an in vivo immunogenic response.

### VII. Examples

### Example 1. Synthesis of ATX-193

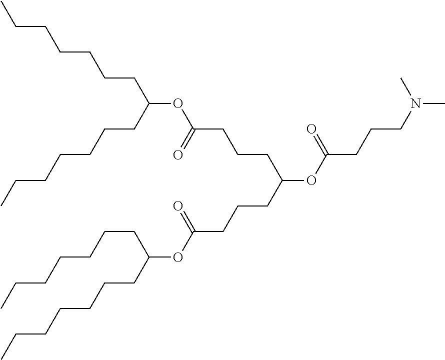 [SMILES: CCCCCCCC(CCCCCCC)OC(=O)CCCC(CCCC(=O)OC(CCCCCCC)CCCCCCC)OC(=O)CCCN(C)C]

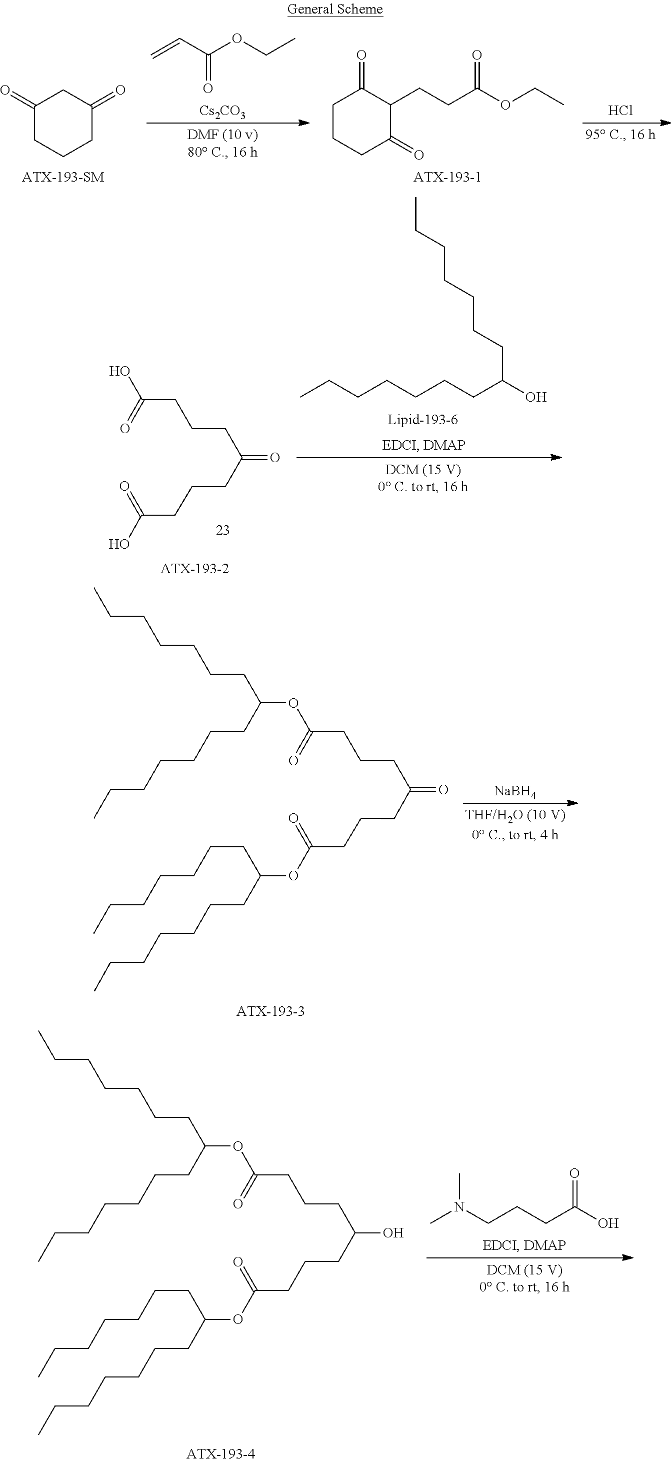

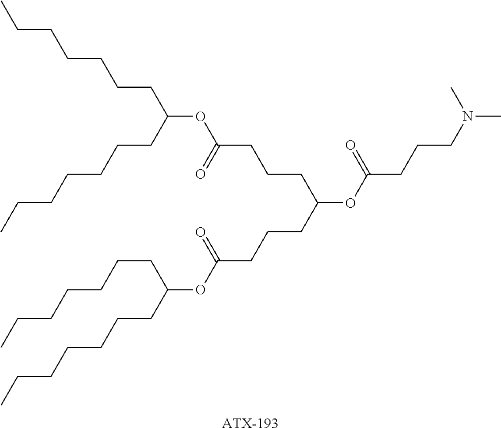

### Synthesis of ATX-193-1

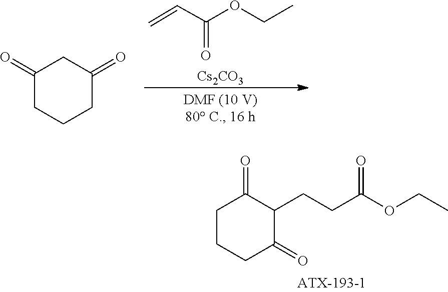

Into a 1-L 3-necked round-bottom flask purged and maintained with an inert atmosphere of nitrogen, was placed 1,3 cyclohexanedione (20 g, 1.00 equiv) and dimethylformamide (200 mL). Ethyl acrylate (21.45 g, 1.20 equiv) and Cs₂CO₃ (35.00 g, 0.60 equiv) were added and the resulting solution was stirred for 16 h at 80° C. The reaction was then quenched by the addition of 600 mL of water/ice. The pH value of the solution was adjusted to 6 with HCl (1 mol/L). The resulting solution was extracted with 2×1 L of ethyl acetate and the organic layers were combined. The combined organic layer was washed with 2 xl L of brine. The organic layer was dried over anhydrous MgSO₄, filtered and concentrated under vacuum. This resulted in 29 g (78%) of ethyl 3-(2,6-dioxocyclohexyl)propanoate as a yellow solid. LCMS (Schimadzu 2020; ELSD A:water/0.05% TFA:B:CH₃CN/0.05% TFA 95:5 to 5:95 A/B at 2.00 min., hold 0.7 min): RT 1.01 min, m/z (Calcd.) 212.10, (found) 213.10 (M+H).

### Synthesis of ATX-193-2

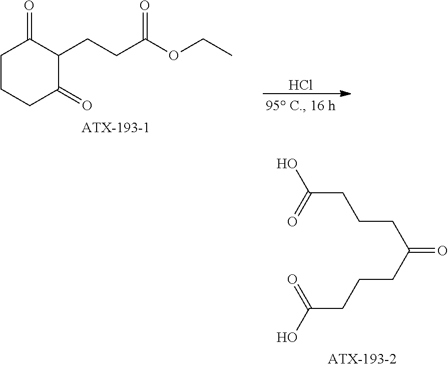

Into a 1-L 3-necked round-bottom flask purged and maintained with an inert atmosphere of nitrogen, was placed ethyl 3-(2,6-dioxocyclohexyl)propanoate (29 g, 1.00 equiv) and HCl (1 mol/L, 300 mL aq). The resulting solution was stirred for 16 h at 95° C. The resulting mixture was concentrated under vacuum. The residue was dissolved in 1 L of EtOAc. The undissolved solids were filtered out. The resulting EA phase was concentrated under vacuum to dry. This resulted in 23 g (crude) of 5-oxononanedioic acid as a yellow solid.

### Synthesis of ATX-193-3

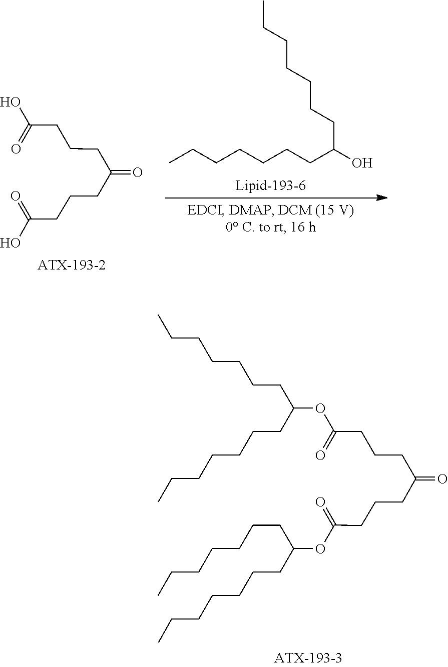

Into a 1-L 3-necked round-bottom flask purged and maintained with an inert atmosphere of nitrogen, was placed 5-oxononanedioic acid (23 g, 1.00 equiv) and DCM (345 mL) at room temperature. This was followed by the addition of pentadecan-8-ol (62 g, 2.2 equiv) and DMAP (13 g, 1.00 equiv) at room temperature, then EDCI (52 g, 2.20 equiv) was added at 0° C. The resulting solution was stirred for 16 h at room temperature. The reaction was then quenched by the addition of 75 mL of HCl aq (1 mol/L). The resulting solution was extracted with 2×1 L of DCM and the organic layers were combined. The organic layer was washed with 2×1 L brine and dried with MgSO₄, filtered and concentrated under vacuum to ˜ 500 mL. To this 100 g silica gel (type: ZCX-2, 100-200 mesh) was added and the mixture was concentrated under vacuum. This silica gel was applied onto a silica gel column (1 Kg, type: ZCX-2, 100-200 mesh) and the product was eluted with PE/EA, gradient from I/O to 80/1. Fractions were collected and the product fraction was concentrated under vacuum. This resulted in 26 g (38%) of 1,9-bis(pentadecan-8-yl) 5-oxononanedioate as a yellow oil. LCMS (Schimadzu 2020; ELSD A:water/0.05% TFA:B:CH₃CN/0.05% TFA 95:5 to 5:95 A/B at 2.00 min., hold 0.7 min): RT 2.99 min, m/z (Calcd.) 623.02, (found) 645.35 (M+Na).

### Synthesis of ATX-193-4

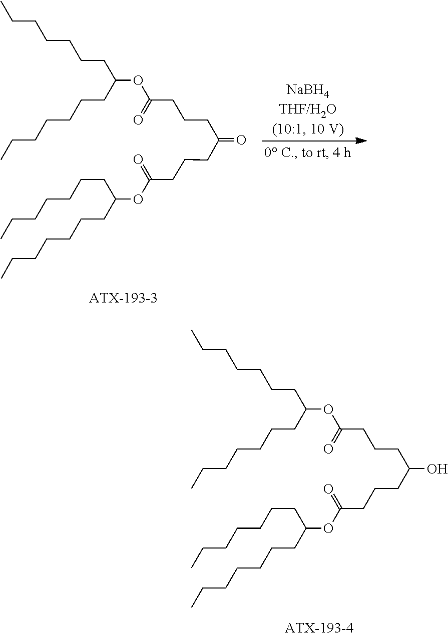 [SMILES: CCCCCCCC(CCCCCCC)OC(=O)CCCC(=O)CCCC(=O)OC(CCCCCCC)CCCCCCC ; "NaBH4THF/H2O(10:1, 10 V)" ; CCCCCCCC(CCCCCCC)OC(=O)CCCC(O)CCCC(=O)OC(CCCCCCC)CCCCCCC]

Into a 500-mL 3-necked round-bottom flask was purged and maintained with an inert atmosphere of nitrogen, 1,9-bis(pentadecan-8-yl) 5-oxononanedioate (18 g, 1.00 equiv) and THF/H₂O (10:1, 180 mL) was added. This was followed by the addition of NaBH₄ (1.08 g, 1.00 equiv) at 0° C. The resulting solution was stirred for 4 h at room temperature. The reaction was then quenched by the addition of 200 mL of water/ice. The resulting solution was extracted with 3×300 mL of ethyl acetate and the organic layers were combined. The organic layer was dried over anhydrous MgSO₄, filtered and concentrated under vacuum. This resulted in 17.3 g (95%) of 1,9-bis(pentadecan-8-yl) 5-hydroxynonanedioate as a light-yellow oil.

### Synthesis of ATX-193-6

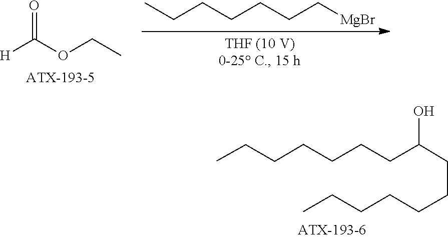

To a 1-L four-neck round-bottle flask with mechanical agitation under N₂, was charged 486 mL of bromo (heptyl)magnesium (1 mol/L) in THF (180 mL) at 25° C. Charge ethyl formate (18.00 g, 1.00 equiv) dropwise with stirring at 0° C. in 30 mins. The resulting solution was stirred for 15 h at room temperature. The reaction was then quenched by the addition of 500 mL saturated NH₄Cl aq. The phases were separated, and the aqueous layer was extracted with 2×500 mL of ethyl acetate. Then combined organic layers were dried over anhydrous MgSO₄, filtered, and concentrated under vacuum. The solid residue was slurry in 60 mL of CH₃CN. The solids were collected by filtration and vacuum dried. This resulted in 50 g (78%) of pentadecan-8-ol as a white powder. This was used as such in the next reaction step.

### Synthesis of ATX-193

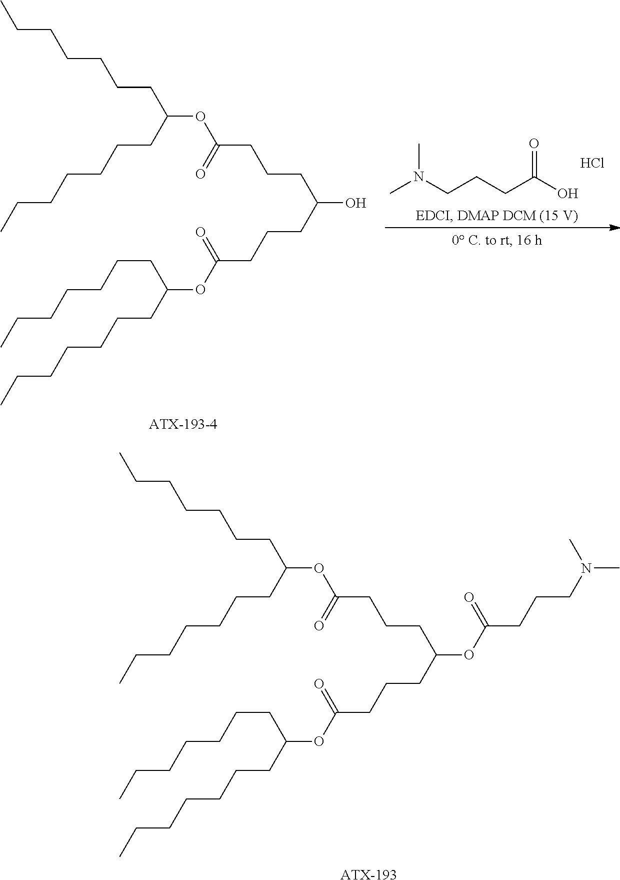

Into a 250-mL 3-necked round-bottom flask purged and maintained with an inert atmosphere of nitrogen, was placed a solution of 1,9-bis(pentadecan-8-yl) 5-hydroxynonanedioate (17.3 g, 1.00 equiv) in DCM (180 mL) at room temperature. 4-(dimethylamino)butanoic acid (5.58 g, 1.20 equiv) and DMAP (0.69 g, 0.20 equiv) were added at room temperature, followed by the addition of EDCI (6.39 g, 1.20 equiv) in portions at 0° C. The resulting solution was stirred for 16 h at room temperature. The reaction was then quenched by the addition of 300 mL of HCl (1 mol/L). The resulting solution was extracted with 2×500 mL of DCM and the organic layers were combined. The organic layer was washed with 2×500 mL brine. The resulting organic layer was concentrated under vacuum and the 40 g crude product obtained was adsorbed on 80 g Silica gel. The residue was purified on a silica gel column (800 g, type: ZCX-2, 100-200 mesh) with DCM/ME, gradient from 100:0 to 90:10. The product containing fractions were concentrated under vacuum. Then the product was dissolved in heptane (300 mL, 20 V), the organic layer was then washed with MeOH/H₂O (3:1) 300 mL (20 V). The heptane phase was concentrated under vacuum. This resulted in 10.5 g (49%) of 1,9-bis(pentadecan-8-yl)₅-[[4-(dimethylamino)butanoyl]oxy] nonanedioate as a colorless oil. ELSD A:water/0.05% TFA:B:CH₃CN/0.05% TFA 95:5 to 5:95 A/B at 2.00 min., hold 0.7 min): RT 2.76 min, m/z (Calcd.) 737.6, (found) 738.5 (M+H); H-NMR: (300 MHz, Chloroform-d, ppm): δ 4.881 (h, 3H), 2.332 (dt, 8H), 2.241 (s, 6H), 1.812 (m, 2H), 1.710-1.413 (m, 16H), 1.282 (s, 40H), 0.952-0.844 (m, 12H).

### Example 2. Synthesis of ATX-200

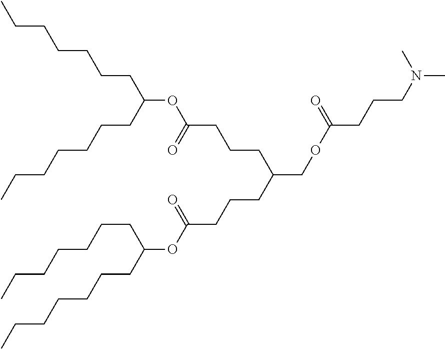 [SMILES: CCCCCCCC(CCCCCCC)OC(=O)CCCC(CCCC(=O)OC(CCCCCCC)CCCCCCC)COC(=O)CCCN(C)C]

General Scheme:

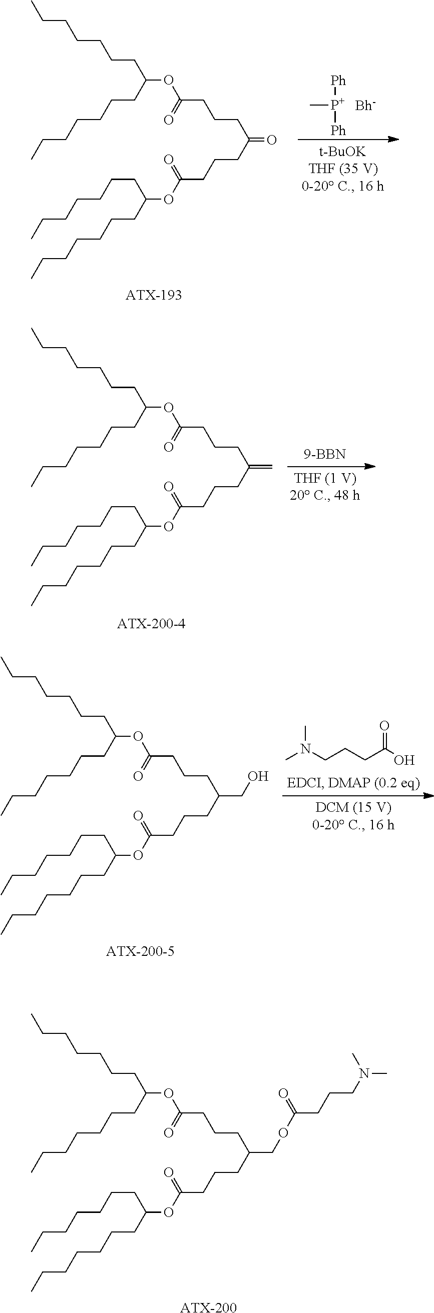

### Synthesis of ATX-200-4

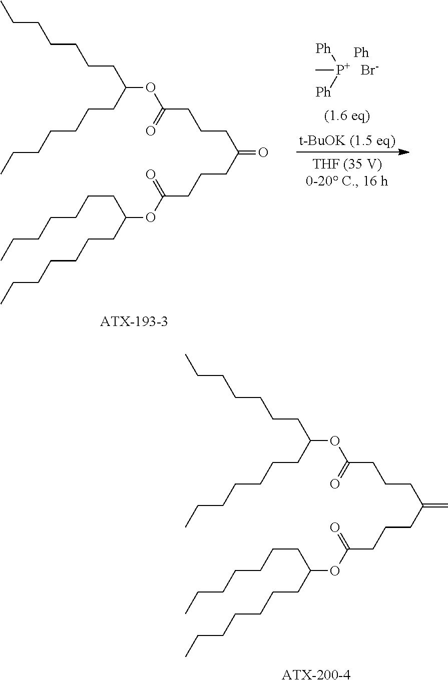

Into a 500-mL 4-necked round-bottom flask purged and maintained with an inert atmosphere of nitrogen, was placed methyltriphenylphosphanium bromide (4540.31 mg, 12.456 mmol, 1.60 equiv, 98%), THF (150.00 mL, 99%). This was followed by the addition of t-BuOK (1323.54 mg, 11.677 mmol, 1.50 equiv, 99%) in several batches at 0° C. in 10 min. To this was added 1,9-bis(pentadecan-8-yl) 5-oxononanedioate (5.00 g, 7.785 mmol, 1.00 equiv, 97%) in THF (25 ml) at 0° C. in 20 min. The resulting solution was stirred for 18 hr at 25° C. The resulting mixture was concentrated. The residue was applied onto a silica gel column with ethyl acetate/petroleum ether (1:50). This resulted in 3.7 g (75.00%) of 1,9-bis(pentadecan-8-yl) 5-methylidenenonanedioate as a colorless oil.

### Synthesis of ATX-200-5

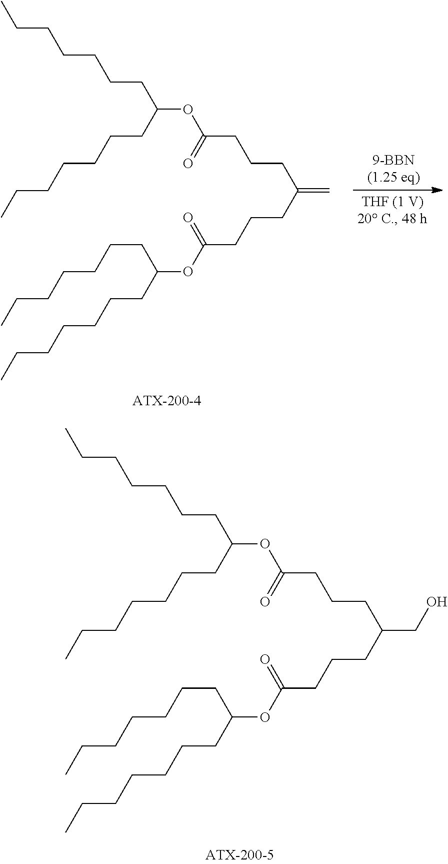

Into a 100-mL 4-necked round-bottom flask purged and maintained with an inert atmosphere of nitrogen, was placed 1,9-bis(pentadecan-8-yl) 5-methylidenenonanedioate (3.7 g, 5.779 mmol, 1.00 equiv, 97%), THE (3.70 mL). This was followed by the addition of 9-BBN (14.90 mL, NaN mmol, 1.25 equiv) dropwise with stirring at 18° C. in 20 min. After the mixture was stirred for 18 h at 18° C., water (1 mL) and 3 N NaOH (5 mL) were added successively. Then, 30% H₂O₂ (10 mL) was added dropwise while maintaining the temperature below 50° C. After being stirred at room temperature for 18 h. The resulting solution was extracted with 2×50 mL of ethyl acetate. The resulting mixture was washed with 3×50 mL of brine. The mixture was dried over anhydrous sodium sulfate and concentrated. The residue was applied onto a silica gel column with ethyl acetate/petroleum ether (1:20). This resulted in 2.6 g (68.29%) of 1,9-bis(pentadecan-8-yl) 5-(hydroxymethyl)nonanedioate as white oil. LCMS (Schimadzu 2020; ELSD A:water/0.05% TFA:B:CH₃CN/0.05% TFA 95:5 to 5:95 A/B at 2.00 min., hold 0.7 min): RT 3.19 min, m/z (Calcd.) 638.5, (found) 661.5 (M+Na).

### Synthesis of ATX-200

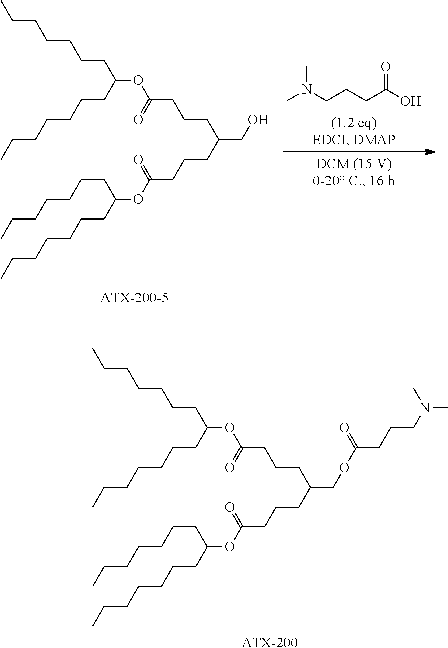

Into a 100-mL 4-necked round-bottom flask purged and maintained with an inert atmosphere of nitrogen, was placed 1,9-bis(pentadecan-8-yl) 5-(hydroxymethyl)nonanedioate (2.60 g, 3.946 mmol, 1.00 equiv, 97%), THE (15.00 mL). This was followed by the addition of 4-(dimethylamino)butanoic acid (633.88 mg, 4.736 mmol, 1.20 equiv, 98%) in several batches at 0° C. in 10 min. To this was added EDCI (926.37 mg, 4.736 mmol, 1.20 equiv, 98%) in several batches at 0° C. in 10 min. To the mixture was added DMAP (98.39 mg, 0.789 mmol, 0.20 equiv, 98%) in several batches at 0° C. in 10 min. The resulting solution was stirred for 18 hr at 18° C. The resulting mixture was concentrated. The residue was applied onto a silica gel column with ethyl DCM:MeOH (30:1). The crude product was purified by Flash-Prep-HPLC with the following conditions (IntelFlash-1): Column, C18 silica gel; mobile phase, 2-Propanol:H₂O=60:40 increasing to 2-Propanol:H₂O=80:20 within 30; Detector, Evaporative light. product was obtained. Then the product was dissolved in heptane (30 mL, 20 V), the organic layer was then washed with MeOH/H₂O (3:1) (20 V). The heptane phase was concentrated under vacuum. This resulted in 1.5 g (50.02%) of 1,9-bis(pentadecan-8-yl) 5-([[4-(dimethylamino)butanoyl]oxy]methyl) nonanedioate as light yellow oil. ELSD A:water/0.05% TFA:B:CH₃CN/0.05% TFA 95:5 to 5:95 A/B at 2.00 min., hold 0.7 min): RT 3.19 min, m/z (Calcd.) 751.67, (found) 752.50 (M+H); H-NMR: (400 MHz, Chloroform-d, ppm): 4.847-4.909 (m, 2H), 4.001-4.015 (m, 2H), 2.241-2.380 (m, 14H), 1.392-1.845 (m, 69H).

### Example 3. Synthesis of ATX-201

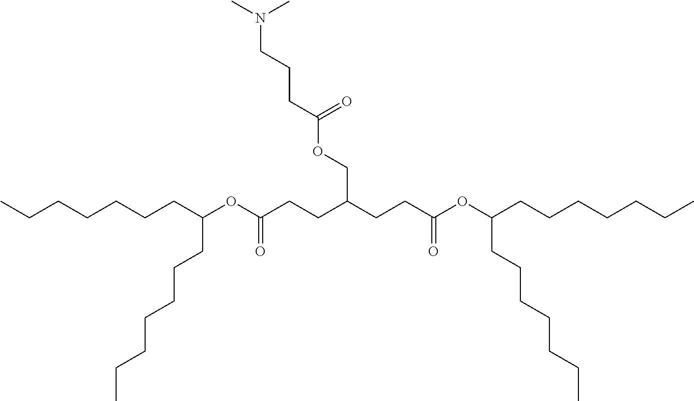 [SMILES: CCCCCCCC(CCCCCCC)OC(=O)CCC(CCC(=O)OC(CCCCCCC)CCCCCCC)COC(=O)CCCN(C)C]

General Scheme.

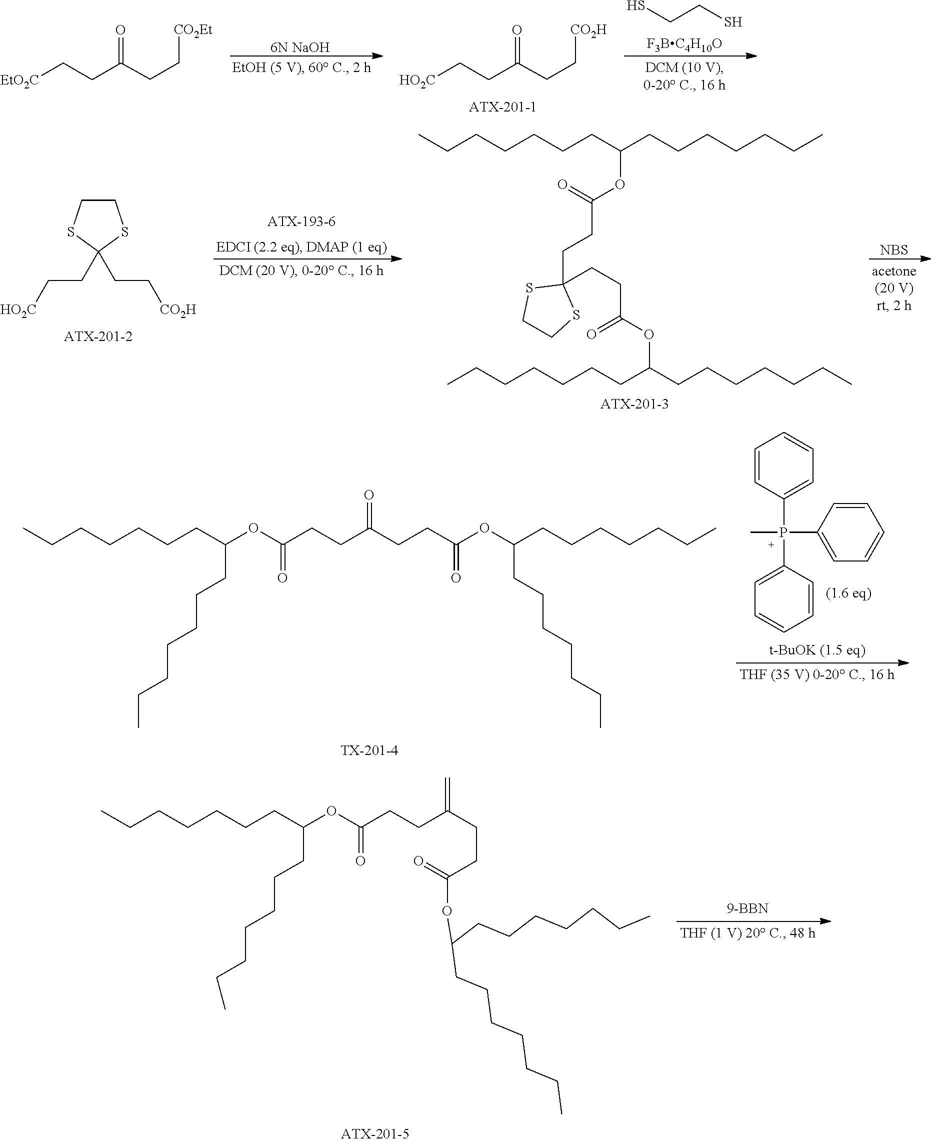

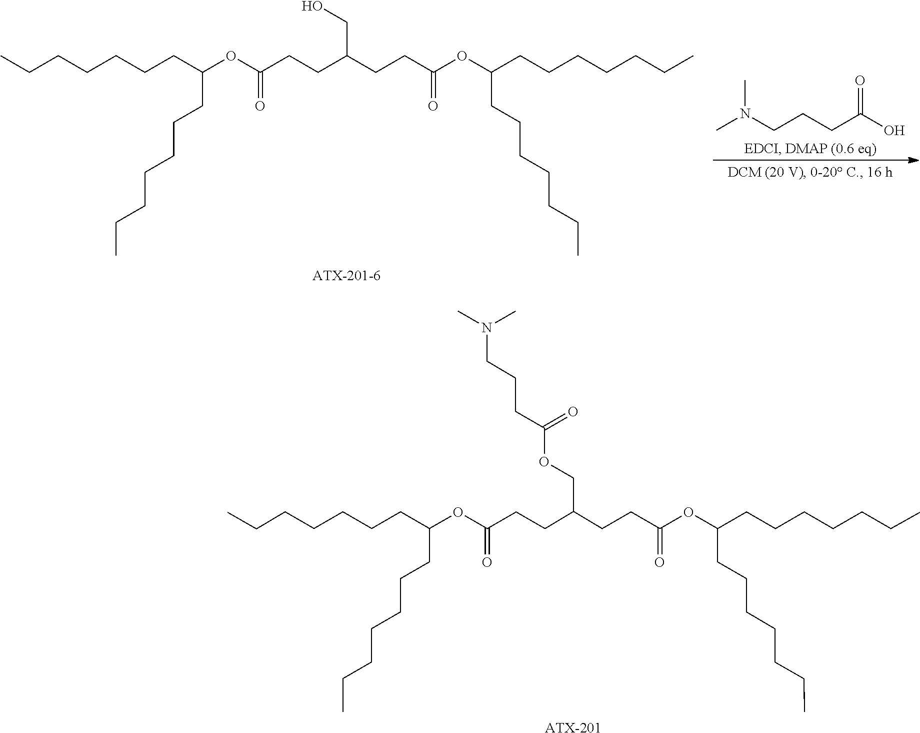

### Synthesis of ATX-201-1

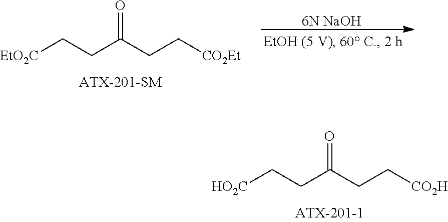 [SMILES: CCOC(=O)CCC(=O)CCC(=O)OCC ; "6N NaOH" ; O=C(O)CCC(=O)CCC(=O)O]

To a three-neck round-bottom flask was added EtOH (25 mL, 5 V) and ATX-201-SM (5 g, 1 eq) at room temperature and stir. 6N NaOH (25 mL, 5 V) was added slowly to the mixture at 0° C. The resulting solution was stirred for 2 h at 60° C., TLC indicated complete consumption of ATX-201-SM. Brine (10 wt. %, 50 mL, 10 V) and DCM (50 mL, 10 V) was added to the mixture and stir for 10 minutes and phase cut, water phase was collected and the pH was adjusted to 3-4 with 3N HCl. The mixture was extracted with DCM (100 mL, 20 V). The organic phase was dried with anhydrous MgSO₄ and then filtered. Concentrated and dried under vacuum to afford the ATX-205-1 (3.2 g, 84.6% yield) as light yellow solid.

### Synthesis of ATX-201-2

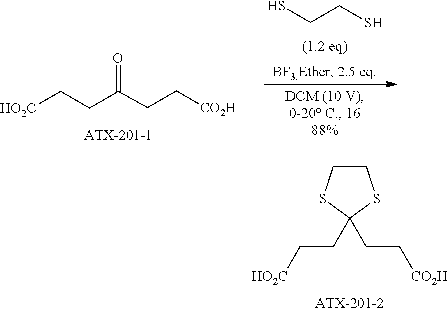

To a three-neck round-bottom flask was added DCM (100 mL, 10 V), ATX-201-1 (3.2 g, 1 eq) and ethane-1,2-dithiol (2.1 g, 1.2 eq) at room temperature. BF₃.Et₂O (2.5 eq) was added slowly to the mixture at 0° C. Resulting solution was stirred for 16 h at 20° C., TLC indicated complete consumption of ATX-201-1. The solid was collected by filtration. The solid was dried under vacuum to afford the ATX-201-2 (4 g, 88% yield) as light yellow solid. This was used as such in the next reaction.

### Synthesis of ATX-201-3

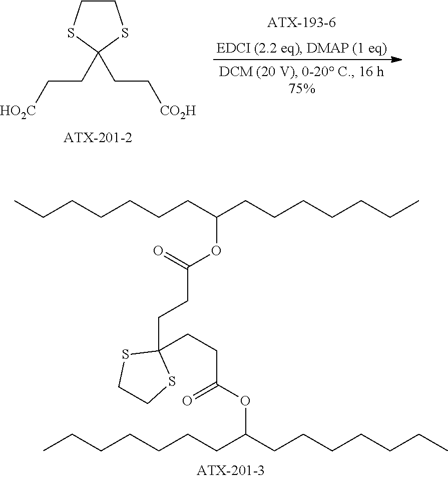 [SMILES: O=C(O)CCC1(CCC(=O)O)SCCS1 ; "EDCI (2.2 eq), DMAP (1 eq)" ; CCCCCCCC(CCCCCCC)OC(=O)CCC1(CCC(=O)OC(CCCCCCC)CCCCCCC)SCCS1]

To a three-neck round-bottom flask was added DCM (80 mL, 20 V), ATX-201-2 (4 g, 1.0 eq), ATX-193-6 (8 g, 2.2 eq) and DMAP (2 g, 1 eq) successively. EDCI (6.7 g, 2.2 eq) was added to the reaction mixture at 0° C. in portions. The resulting solution was stirred for 16 h at 20° C., TLC indicated complete consumption of ATX-201-2. The reaction system was quenched with 10% citric acid solution (40 mL, 10 V). Collected organic phase, the organic phase was washed with 10% citric acid solution (40 mL, 10 V), and washed with brine (40 mL, 10 V). The organic phase was dried with anhydrous MgSO₄ and then filtered. The crude product was adsorbed on 20 g of silica gel and purified on a 100 g silica gel column (type: ZCX-2, 100-200 mesh, 8.00 w./w.) eluting with PE/EA gradient from 100:0 to 99:1. Qualified fractions were combined, concentrated and dried under vacuum to afford the ATX-201-3 (8 g, 75% yield) as colorless oil. ¹H NMR (300 MHz, Chloroform-d) δ 4.86 (p, J=6.2 Hz, 2H), 3.26 (s, 4H), 2.67-2.56 (m, 4H), 2.30-2.15 (m, 4H), 1.50 (t, J=6.3 Hz, 8H), 1.28 (d, J=11.2 Hz, 41H), 0.88 (d, J=6.3 Hz, 12H).

### Synthesis of ATX-201-4

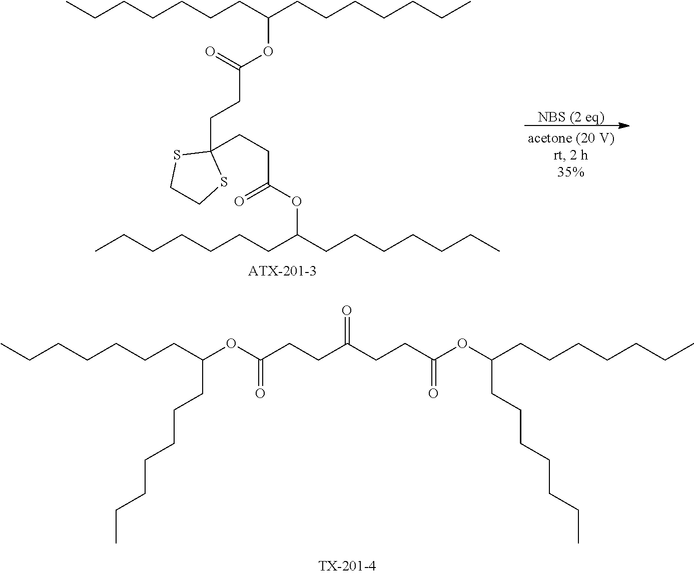

To a three-neck round-bottom flask was added acetone (160 mL, 20 V), ATX-201-3 (8 g, 1.0 eq) successively. NBS (4.25 g, 2 eq) was added to the reaction mixture at 0° C. with portions. The resulting solution was stirred for 2 h at room temperature, TLC indicated complete consumption of ATX-201-3. The solvent was removed under reduced pressure. The crude product was adsorbed on 20 g silica gel and purified on a 100 g of silica gel (type: ZCX-2, 100-200 mesh, 8.00 w./w.) column, using combi-flash system. Products was eluted with PE/EA gradient from 100:0 to 97:3. Qualified products were pooled and concentrated under vacuum to afford the ATX-201-4 (2.5 g, 35% yield) as colorless oil. ¹H NMR (400 MHz, Chloroform-d) δ 4.84 (p, J=6.3 Hz, 2H), 2.76 (t, J=6.7 Hz, 4H), 2.59 (t, J=6.7 Hz, 4H), 1.50 (q, J=6.4, 6.0 Hz, 8H), 1.31-1.21 (m, 40H), 0.91-0.84 (m, 12H).

### Synthesis of ATX-201-5

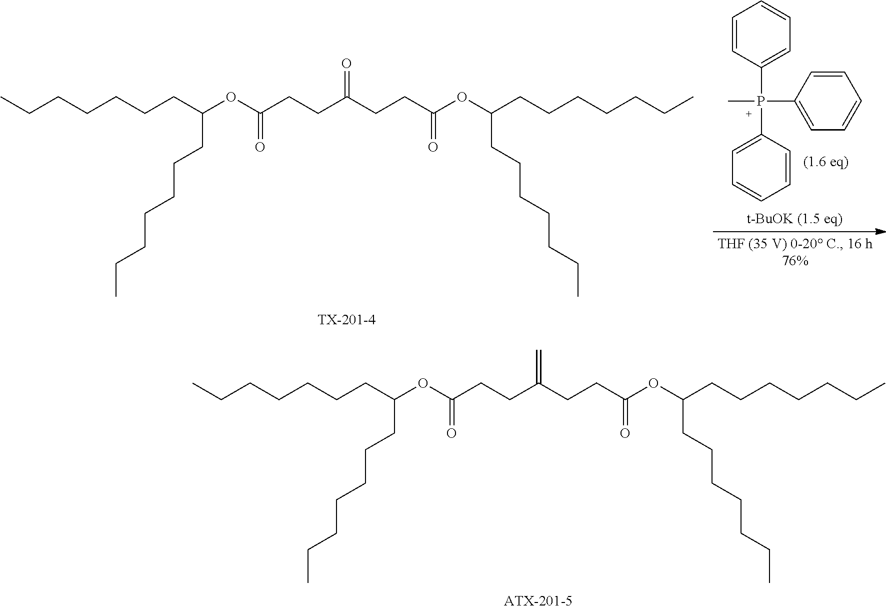

Into a 500-mL 4-necked round-bottom flask purged and maintained with an inert atmosphere of nitrogen, was placed methyltriphenylphosphanium bromide (1.9 g, 1.6 eq), THE (75 mL, 30 V). This was followed by the addition of t-BuOK (0.7 g, 1.5 eq) in several batches at 0° C. in 10 min. To this was added ATX-201-4 (2.5 g, 1 eq) in THE (25 ml) at 0° C. in 20 min. The resulting solution was stirred for 18 hr at 25° C. The resulting mixture was concentrated. The product was adsorbed on 5 g of silica gel and purified on a 25 g of silica gel (type: ZCX-2, 100-200 mesh, 8.00 w./w.) column on Combi-Flash system by eluting with PE/EA gradient from 100:0 to 99:1. Qualified products were combined, concentrated and dried under vacuum to afford the ATX-201-5 (1.9 g, 76% yield) as colorless oil. ¹H NMR (300 MHz, Chloroform-d) δ 4.88 (p, J=6.2 Hz, 2H), 4.78 (s, 2H), 2.47 (ddd, J=8.5, 6.2, 1.8 Hz, 4H), 2.37 (dd, J=8.6, 5.9 Hz, 4H), 1.53 (s, 8H), 1.27 (, 40H), 0.88 (m, 12H).

### Synthesis of ATX-201-6

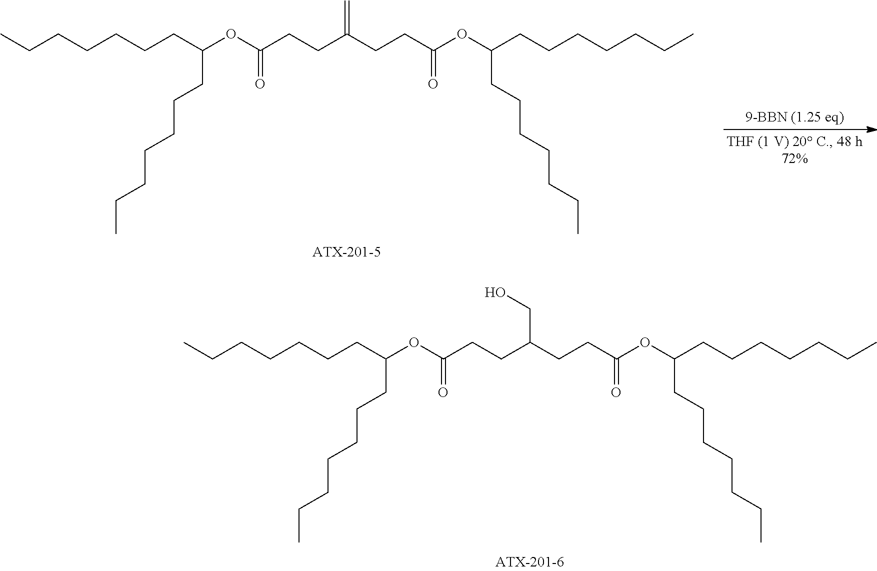

To a three-neck round-bottom flask was added ATX-201-5 (1.9 g, 1 eq), THE (3.70 mL) successively. This was followed by the addition of 0.5 mol 9-BBN in THE (8 mL, 1.25 eq) dropwise with stirring at 18° C. in 20 min. After the mixture was stirred for 18 h at 18° C., water (0.47 mL, 0.25 V) and 3 N NaOH (2.8 mL, 1.5 V) were added successively. Then, 30% H₂O₂ (4.75 ml, 2.5 V) was added dropwise while maintaining the temperature below 50° C. After being stirred at room temperature for 18 h, the resulting solution was extracted with 2×20 mL of ethyl acetate. The combined organic phase was washed with 3×20 mL of brine. The mixture was dried over anhydrous sodium sulfate and filtered. The product was adsorbed on 5 g of silica gel and purified on a 25 g of silica gel (type: ZCX-2, 100-200 mesh, 8.00 w./w.) column on Combi-Flash system by eluting with PE. Qualified products were combined, concentrated and dried under vacuum to afford ATX-201-6 (1.4 g, 76% yield) as colorless oil. ¹H NMR (300 MHz, Chloroform-d) δ 4.87 (p, J=6.3 Hz, 2H), 4.12 (q, J=7.1 Hz, 1H), 3.52 (d, J=4.8 Hz, 2H), 2.40-2.27 (m, 4H), 1.75-1.61 (m, 3H), 1.51 (d, J=6.4 Hz, 9H), 1.27 (t, J=3.7 Hz, 40H), 1.23 (m, 40H), 0.87 (m, 12H).

### Synthesis of ATX-201

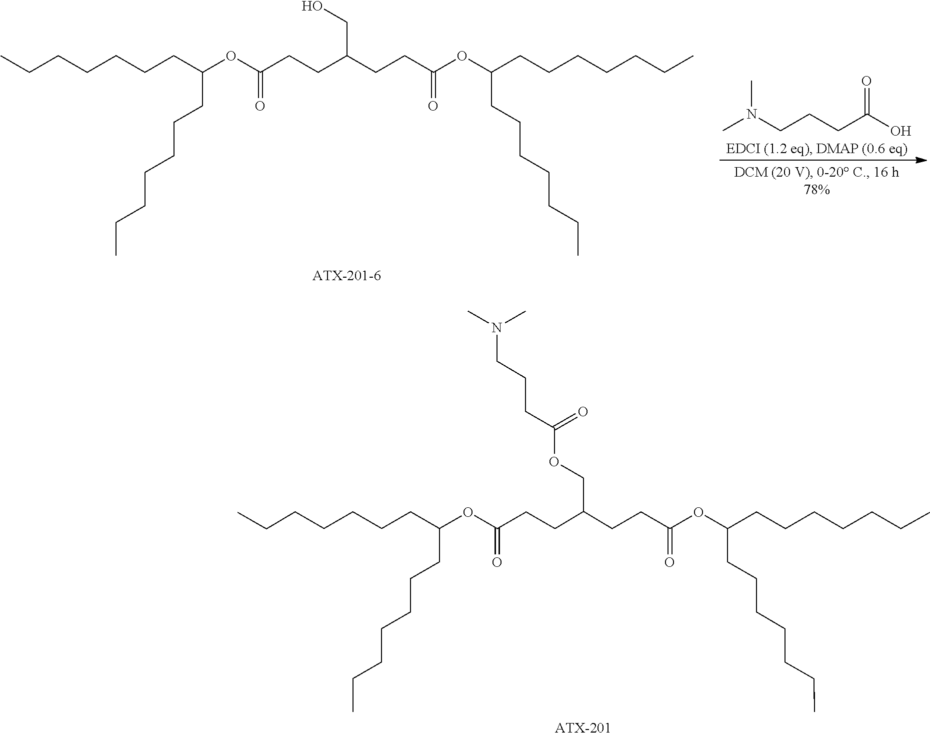

To a three-neck round-bottom flask was added DCM (28 mL, 20 V), ATX-201-6 (1.4 g, 1.0 eq), 4-(dimethylamino)butanoic acid (380 mg, 1 eq) and DMAP (168 mg, 0.6 eq) successively. EDCI (526 mg, 1.2 eq) was added to the reaction mixture at 0° C. with portions. The resulting solution was stirred for 16 h at 20° C., TLC indicated complete consumption of 4-(dimethylamino)butanoic acid. The reaction mixture was quenched with 10% citric acid solution (14 mL, 10 V). Organic phase was collected and washed with 10% citric acid solution (14 mL, 10 V), and washed with brine (14 mL, 10 V). The organic phase was dried with anhydrous MgSO₄ and then filtered. The product was adsorbed on 5 g of silica gel and purified on a 25 g of silica gel (type: ZCX-2, 100-200 mesh, 8.00 w./w.) column on Combi-Flash system by eluting with DCM/MeOH gradient from 100:0 to 95:5. Qualified products were combined, concentrated and dried under vacuum to afford ATX-201 (1.3 g, 78% yield) as light-yellow oil. ELSD A:water/0.05% TFA:B:CH3CN/0.05% TFA 95:5 to 5:95 A/B at 2.00 min., hold 0.7 min): RT 3.19 min, m/z (Calcd.) 723.6, (found) 724.7 ¹H NMR (300 MHz, Chloroform-d) δ 4.831 (p, J=6.2 Hz, 2H), 4.013 (d, J=4.5 Hz, 2H), 3.000-2.881 (m, 2H), 2.70 (s, 6H), 2.464 (t, J=6.7 Hz, 2H), 2.310 (t, J=7.5 Hz, 4H), 2.112 (dq, J=13.7, 6.8 Hz, 2H), 1.653 (t, J=7.2 Hz, 5H), 1.492 (d, J=6.3 Hz, 8H), 1.242 (m, 40H), 0.910-0.762 (m, 12H).

### Example 4. Synthesis of ATX-202

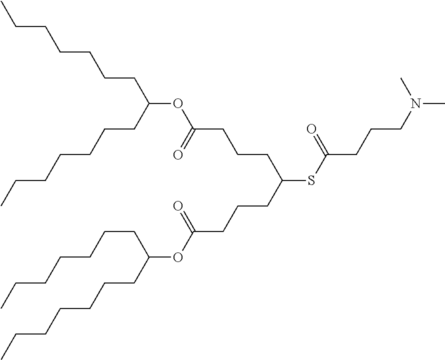 [SMILES: CCCCCCCC(CCCCCCC)OC(=O)CCCC(CCCC(=O)OC(CCCCCCC)CCCCCCC)SC(=O)CCCN(C)C]

General Scheme:

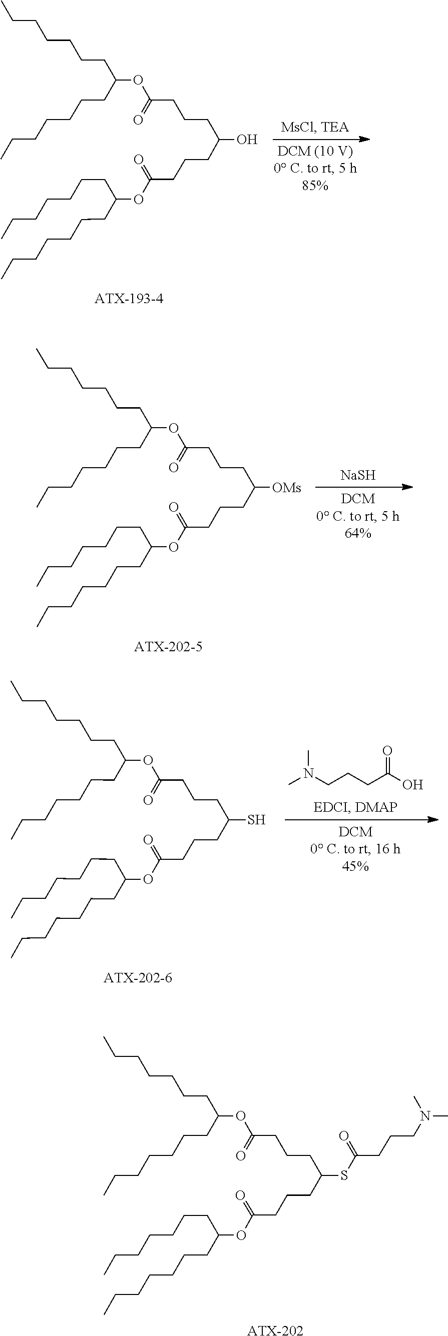

### Synthesis of ATX-202-5

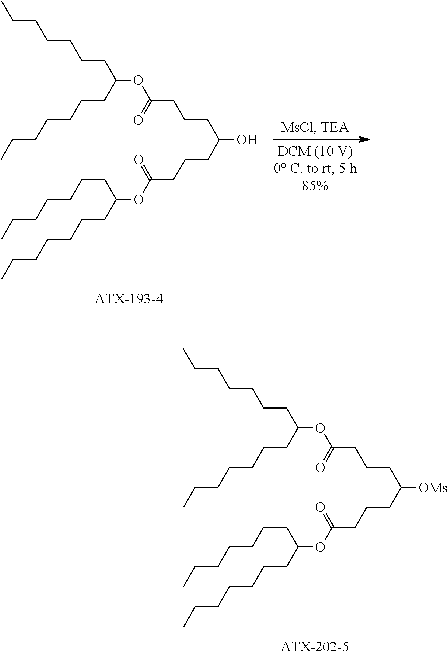 [SMILES: CCCCCCCC(CCCCCCC)OC(=O)CCCC(O)CCCC(=O)OC(CCCCCCC)CCCCCCC ; CCCCCCCC(CCCCCCC)OC(=O)CCCC(CCCC(=O)OC(CCCCCCC)CCCCCCC)OS(C)(=O)=O]

To a 250-mL four-neck round-bottle flask with mechanical agitation under N₂, was charged 5 g 1,9-bis(pentadecan-8-yl) 5-hydroxynonanedioate in DCM (50 mL) at 0° C. This was followed by the addition of TEA (1.6 g, 2 equiv) and MsCl (1.35 g, 1.5 equiv) dropwise with stirring at 0° C. The resulting solution was stirred for 5 h at room temperature. The reaction was then quenched by the addition of 100 mL of H₂O. The phases were separated, and the aqueous layer was extracted with 1×100 mL of DCM. Then combined organic layers; the solvent was dried over anhydrous sodium sulfate, filtered and concentrated under vacuum. This resulted in 4.8 g (85%) of di(pentadecan-8-yl) 5-((methylsulfonyl)oxy)nonanedioate. LCMS (Schimadzu 2020; ELSD A:water/0.05% TFA:B:CH₃CN/0.05% TFA 95:5 to 5:95 A/B at 2.00 min., hold 0.7 min): RT 4.76 min, m/z (Calcd.) 703.12, (found) 725.3 (M+Na).

### Synthesis of ATX-202-6

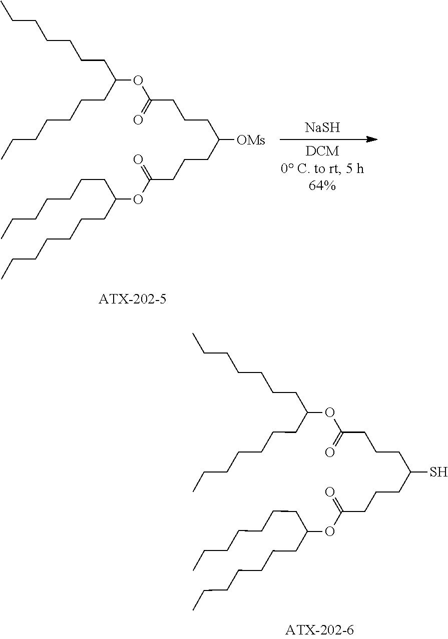 [SMILES: CCCCCCCC(CCCCCCC)OC(=O)CCCC(CCCC(=O)OC(CCCCCCC)CCCCCCC)OS(C)(=O)=O ; "NaSH" ; CCCCCCCC(CCCCCCC)OC(=O)CCCC(S)CCCC(=O)OC(CCCCCCC)CCCCCCC]

Into a 100-mL 3-necked round-bottom flask purged and maintained with an inert atmosphere of nitrogen, was placed 1,9-bis(pentadecan-8-yl) 5-oxononanedioate (4.8 g, 1.00 equiv) in DMF (48 mL). This was followed by the addition of NaHS (2 g, 5.00 equiv) at 0° C. The resulting solution was stirred for 5 h at room temperature. The reaction was then quenched by the addition of 200 mL of water/ice. The resulting solution was extracted with 3×100 mL of ethyl acetate and the organic layers combined. The mixture was dried over anhydrous sodium sulfate and concentrated under vacuum. This resulted in 2.8 g (64%) of di(pentadecan-8-yl) 5-mercaptononanedioate as a light yellow oil. LCMS (Schimadzu 2020; ELSD A:water/0.05% TFA:B:CH₃CN/0.05% TFA 95:5 to 5:95 A/B at 2.00 min., hold 0.7 min): RT 4.84 min, m/z (Calcd.) 640.5, (found) 663.4 (M+Na+H).

### Synthesis of ATX-202

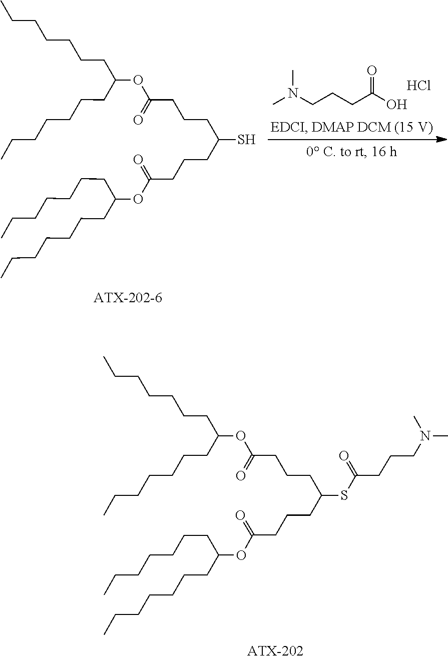

Into a 100-mL 3-necked round-bottom flask purged and maintained with an inert atmosphere of nitrogen, was placed a solution of di(pentadecan-8-yl) 5-mercaptononanedioate (2.8 g, 1.00 equiv) in DCM (28 mL). 4-(dimethylamino)butanoic acid (0.87 g, 1.20 equiv), DMAP (0.1 g, 0.20 equiv) were added, followed by the addition of EDCI (0.95 g, 1.20 equiv) in portions at 0° C. The resulting solution was stirred for 16 h at room temperature. The reaction was then quenched by the addition of 100 mL of HCl (1 mol/L). The resulting solution was extracted with 2×100 mL of DCM and the organic layers combined. The resulting mixture was washed with 2×100 mL of brine. The resulting mixture was concentrated under vacuum and 6 g crude product was obtained. The product was dissolved in 30 mL DCM and 10 g Silica gel (type: ZCX-2, 100-200 mesh) was added. The mixture was concentrated under vacuum. The residue was applied onto atmospheric silica gel column (800 g, type: ZCX-2, 100-200 mesh) with DCM/ME, gradient from I/O to 30/1 and collect product eluent (from 50/1-30/1). The collected product phase was concentrated under vacuum. Then the product was dissolved in heptane (30 mL, 20 V), the organic layer was then washed with MeOH/H₂O (3:1) 30 mL (20 V). The heptane phase was concentrated under vacuum. This resulted in 1.3 g (45%) of 1,9-bis(pentadecan-8-yl)₅-[[4-(dimethylamino)butanoyl]oxy] nonanedioate as a colorless oil. LCMS (Schimadzu 2020; ELSD A:water/0.05% TFA:B:CH₃CN/0.05% TFA 95:5 to 5:95 A/B at 2.00 min., hold 0.7 min): RT 2.83 min, m/z (Calcd.) 754.25, (found) 754.45 (M); ¹H-NMR (300 MHz, Chloroform-d, ppm): δ 4.83-4.87 (m, 2H), 3.51-3.54 (s, 1H), 2.55-2.60 (m, 2H), 2.21-2.30 (m, 12H), 1.40-1.91 (m, 19H), 1.11-1.30 (m, 41H), 1.28 (s, 40H), 0.82-0.91 (m, 12H).

### Example 5. Synthesis of ATX-209

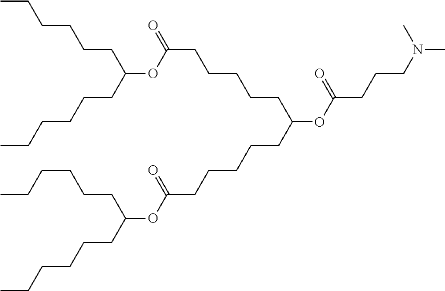 [SMILES: CCCCCCC(CCCCCC)OC(=O)CCCCCC(CCCCCC(=O)OC(CCCCCC)CCCCCC)OC(=O)CCCN(C)C]

General Scheme:

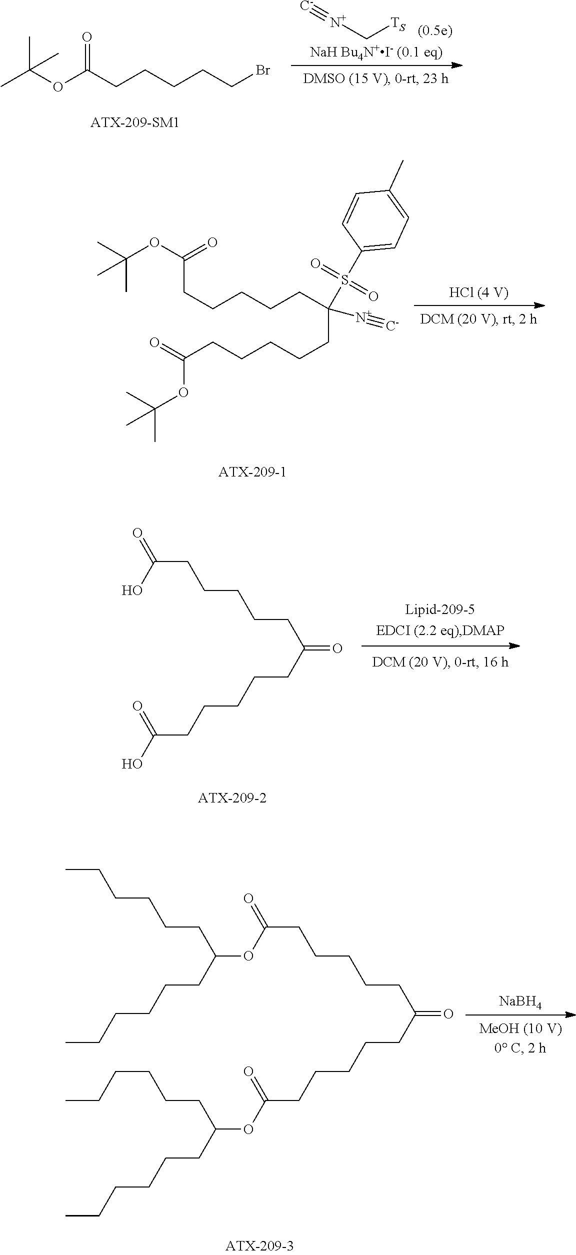

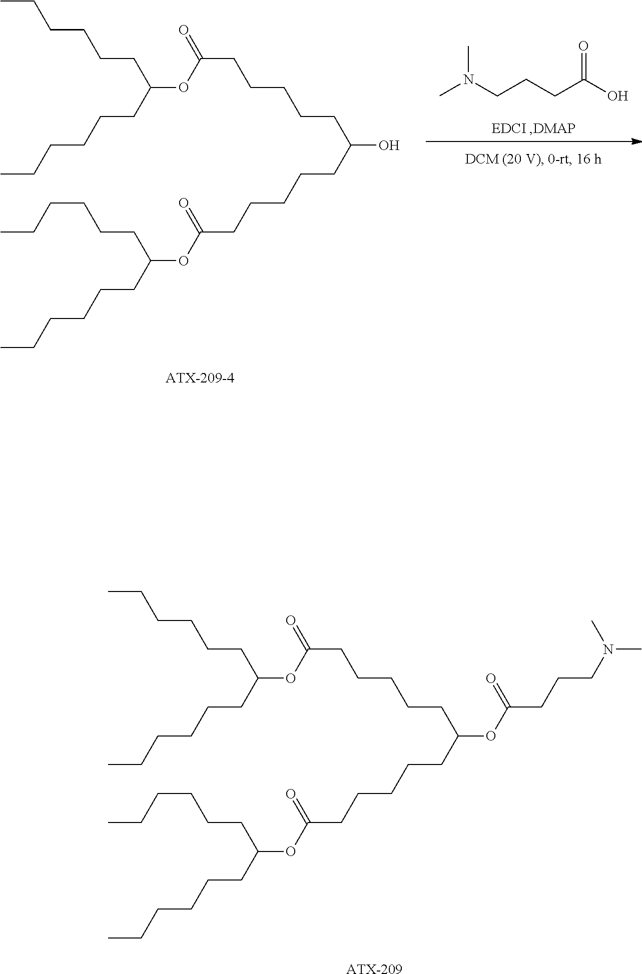

### Synthesis of ATX-209-1

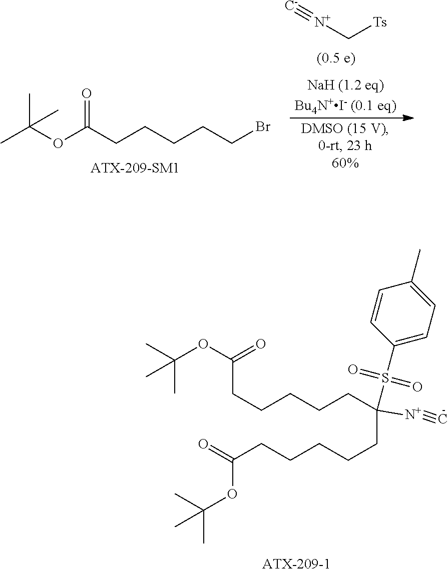 [SMILES: [C-]#[N+]CS(=O)(=O)c1ccc(C)cc1 ; CC(C)(C)OC(=O)CCCCCBr ; C#[N+]C(CCCCCC(=O)OC(C)(C)C)(CCCCCC(=O)OC(C)(C)C)S(=O)(=O)c1ccc(C)cc1]

To a three-neck round-bottom flask was added DMSO (38 mL, 15 V), ATX-209-SM1 (2.5 g, 1 eq) and 1 ((isocyanomethyl)sulfonyl)-4-methylbenzene (1 g, 0.5 eq) at room temperature. To the mixture was added slowly NaH (0.30 g, 1.2 eq) and tetrabutylammonium iodide (0.37 g, 0.1 eq) successively at 0° C. The resulting solution was stirred for 2 h at 60° C., TLC indicated complete consumption of ATX-209-SM1. The reaction was then quenched by the addition of 25 mL of water. The solution was extracted with DCM (3×25 mL). The organic phase was washed with 2×25 mL of saturated brine. The organic phase was dried over anhydrous magnesium sulfate. The organic phase was dried with anhydrous MgSO₄ and then filtered, concentrated and dried under vacuum to afford the ATX-209-1 (3.2 g, 60% yield) as colorless oil. LCMS (Schimadzu 2020; ELSD A:water/0.05% TFA:B:CH₃CN/0.05% TFA 95:5 to 5:95 A/B at 2.00 min., hold 0.7 min): RT 0.84 min, m/z (Calcd.) 535.30, (found) 558.20 (M+Na).

### Synthesis of ATX-209-2

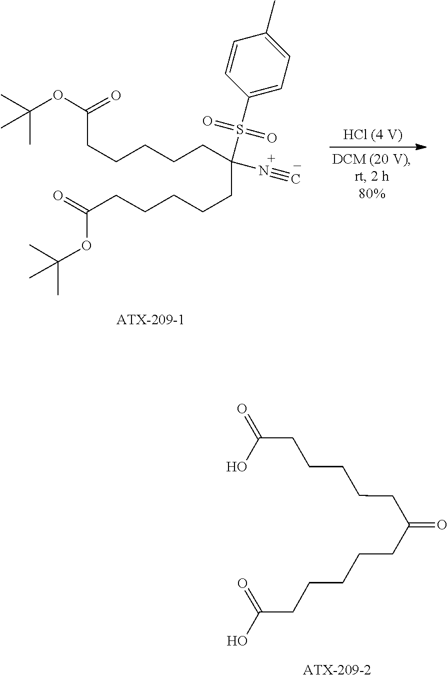

To a three-neck round-bottom flask was added DCM (30 mL, 10 V), ATX-209-1 (3 g, 1 eq) one portion at room temperature. HCl (6 ml, 2 V) was added slowly to the mixture at 0° C., The resulting solution was stirred for 2 h at 0° C., TLC indicated complete consumption of ATX-209-1. The reaction was then quenched by the addition of 30 mL of sodium bicarbonate. The organic phase was washed with 2×30 mL of saturated brine. The organic phase was dried with anhydrous MgSO₄ and then filtered. To the filtrate was added 5 g of silica gel (type: ZCX-2, 100-200 mesh, 2.00 w./w.), concentrated to no fraction under vacuum while maintaining the temperature below 35° C. Charged 25 g of silica gel (type: ZCX-2, 100-200 mesh, 8.00 w./w.) to the column, followed by the last step prepared dry silica gel which absorbed the reaction mixture. Using combi-flash to purify the product. Eluted with DCM/MeOH (volume ratio). (gradient from 100:0 to 20:1 and collected every 100±50 mL). Took sample for TLC analysis. Combined qualified products. Concentrated to dry under vacuum to afford the ATX-209-2 (1.15 g, 80% yield) as a white solid. ¹H NMR (300 MHz, DMSO-d6) δ 2.36 (t, J=7.2 Hz, 4H), 2.16 (t, J=7.3 Hz, 4H), 1.43 (8H), 1.21-1.12 (m, 4H).

### Synthesis of ATX-209-3

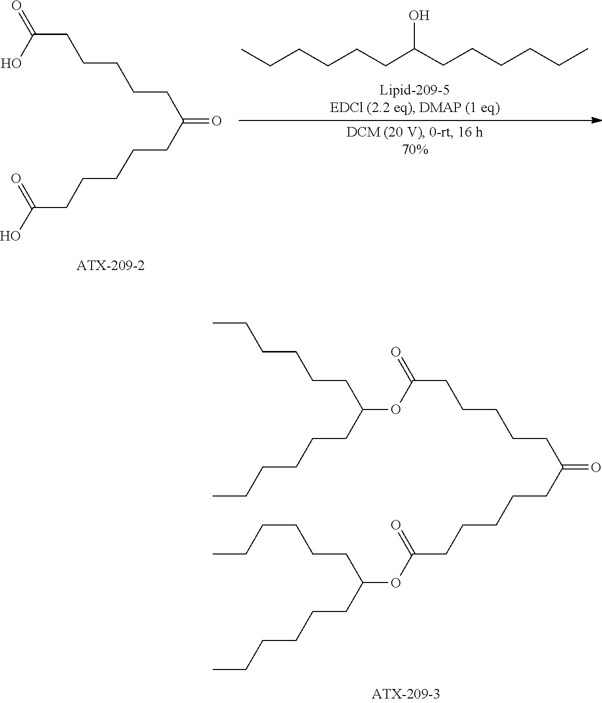 [SMILES: O=C(O)CCCCCC(=O)CCCCCC(=O)O ; CCCCCCC(O)CCCCCC ; CCCCCCC(CCCCCC)OC(=O)CCCCCC(=O)CCCCCC(=O)OC(CCCCCC)CCCCCC]

To a three-neck round-bottom flask was added DCM (20 mL, 20 V), ATX-209-2 (1 g, 1.0 eq), ATX-209-5 (1.71 g, 2.2 eq) and DMAP (0.47 g, 1 eq) successively. EDCI (1.63 g, 2.2 eq) was added to the reaction mixture at 0° C. with portions. The resulting solution was stirred for 16 h at 20° C., TLC indicated completed consumption of ATX-209-2. The reaction system was quenched with 10% citric acid solution (10 mL, 10 V). Collected the organic phase, the organic phase was washed with 10% citric acid solution (10 mL, 10 V), and washed with brine (10 mL, 10 V). The organic phase was dried with anhydrous MgSO₄ and then filtered. To the filtrate was added 5 g of silica gel (type: ZCX-2, 100-200 mesh, 2.00 w./w.), concentrated to no fraction under vacuum while maintaining the temperature below 35° C. Charged 25 g of silica gel (type: ZCX-2, 100-200 mesh, 8.00 w./w.) to the column, followed by the last step prepared dry silica gel which absorbed the reaction mixture. Using combi-flash to purify the product. Eluted with PE/EA (volume ratio). (gradient from 100:0 to 50:1 collect every 20±10 ml). Took sample for TLC analysis. Combine qualified products. Concentrated to dry under vacuum to afford the ATX-209-3 (1.68 g, 70% yield) as colorless oil. LCMS (Schimadzu 2020; ELSD A:water/0.05% TFA:B:CH₃CN/0.05% TFA 95:5 to 5:95 A/B at 5.00 min., hold 0.7 min): RT 4.83 min, m/z (Calcd.) 622.55, (found) 645.3 (M+Na).

### Synthesis of ATX-209-4

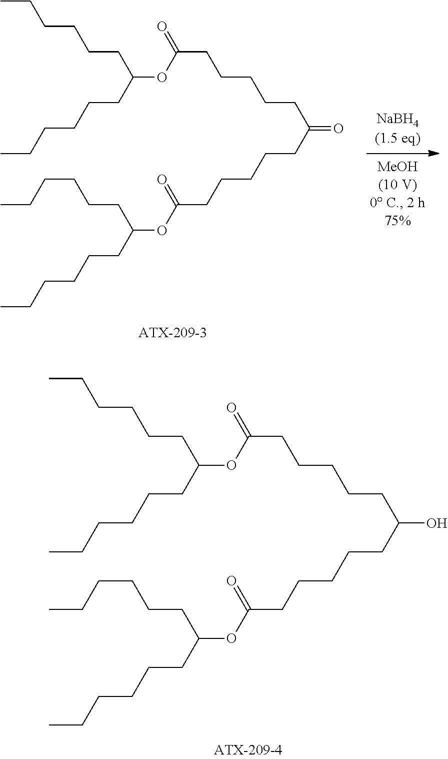 [SMILES: CCCCCCC(CCCCCC)OC(=O)CCCCCC(=O)CCCCCC(=O)OC(CCCCCC)CCCCCC ; CCCCCCC(CCCCCC)OC(=O)CCCCCC(O)CCCCCC(=O)OC(CCCCCC)CCCCCC]

To a 100 ml three-neck flask was added MeOH (20 ml, 10V), ATX-209-3 (2 g, 1 eq) at room temperature. NaBH₄ (0.18 g, 1.5 eq) was added to the reaction mixture at 0° C. with portions. The resulting solution was stirred for 2 h at 0° C., TLC indicated complete consumption of ATX-209-3. The reaction was then quenched by the addition of 20 mL of water. The system was extracted again with METB (2×10 ml, 10 V). The organic phase was dried with anhydrous MgSO₄ and then filtered. Concentrated to dry under vacuum to afford the ATX-209-3 (1.5 g, 75% yield) as colorless oil. LCMS (Schimadzu 2020; ELSD A:water/0.05% TFA:B:CH ₃CN/0.05% TFA 95:5 to 5:95 A/B at 5.50 min., hold 0.7 min): RT 4.83 min, m/z (Calcd.) 624.57, (found) 647.35 (M+Na).

### Synthesis of ATX-209-5

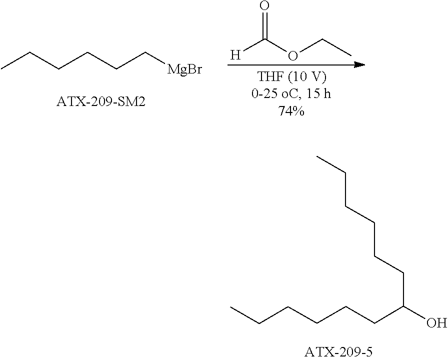

To a 100 ml four-neck round-bottle flask with mechanical agitation under N₂ was added ATX-209-SM2 (1 mol/L, 31 ml) in THE (10 mL) at 25° C. Ethyl formate (1 g, 1.00 eq) was added dropwise with stirring at 0° C. The resulting solution was stirred for 15 h at room temperature. The reaction was then quenched by the addition of NH₄Cl solution (20 mL, 20 V). The phases were separated, and the aqueous layer was extracted with ethyl acetate (2×20 mL). Then organic layers were combined. The solvent was dried over anhydrous sodium sulfate. Filtered and concentrated under vacuum. The residue was slurred with 6 mL of ACN. The solids were collected by filtration. This resulted in ATX-209-5 (2 g, 74% yield) as white powder.

### Synthesis of ATX-209

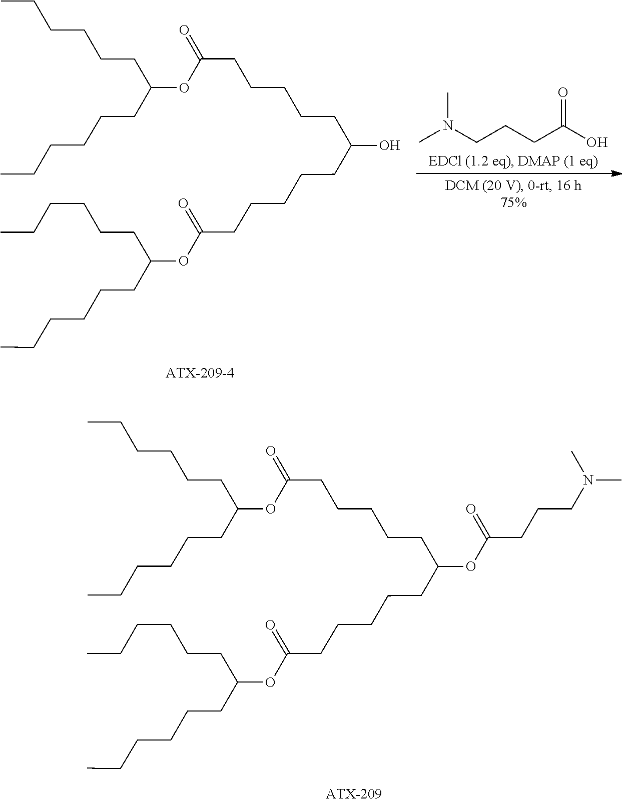

To a three-neck round-bottom flask was added DCM (30 mL, 20 V), 4-(dimethylamino)butanoic acid (0.45 g, 1.1 eq), ATX-209-4 (1.5 g, 1 eq) and DMAP (0.29 g, 1 eq) successively. EDCI (0.60 g, 1.3 eq) was added to the reaction mixture at 0° C. with portions. The resulting solution was stirred for 16 h at 20° C., TLC indicated complete consumption of ATX-209-4. The reaction system was quenched with 10% citric acid solution (15 mL, 10 V). Collected organic phase, the organic phase was washed with 10% citric acid solution (15 mL, 10 V), and washed with brine (15 mL, 10 V). The organic phase was dried with anhydrous MgSO₄ and then filtered. To the filtrate was added 5 g of silica gel (type: ZCX-2, 100-200 mesh, 2.00 w./w.), concentrated to no fraction under vacuum while maintaining the temperature below 35° C. Charged 25 g of silica gel (type: ZCX-2, 100-200 mesh, 8.00 w./w.) to the column, followed by the last step prepared dry silica gel which absorbed the reaction mixture. Using combi-flash to purify the product. Elute with PE/EA (volume ratio). (gradient from 100:0 to 50:1 collect every 20±10 ml). Take sample for TLC analysis. Combine qualified products. Concentrated to dry under vacuum to afford the ATX-209 (1.2 g, 75% yield) as colorless oil. ELSD A:water/0.05% TFA:B:CH₃CN/0.05% TFA 95:5 to 5:95 A/B at 5.50 min., hold 0.7 min): RT 4.83 min, m/z (Calcd.) 737.5, (found) 738.3 (M+H); ¹H NMR (300 MHz, Chloroform-d) δ 4.860 (t, J=6.2 Hz, 3H), 2.370-2.201 (m, 14H), 1.801 (q, J=7.4 Hz, 2H), 1.611-1.470 (m, 16H), 1.272 (m, 40H), 0.920-0.821 (m, 12H).

### Example 6. Synthesis of ATX-210

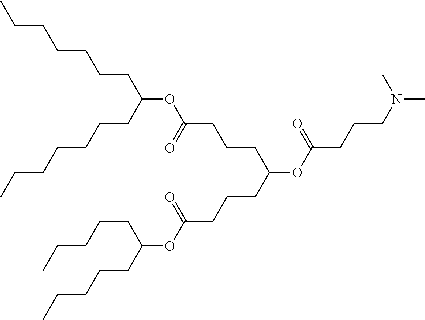 [SMILES: CCCCCCCC(CCCCCCC)OC(=O)CCCC(CCCC(=O)OC(CCCCC)CCCCC)OC(=O)CCCN(C)C]

General Scheme:

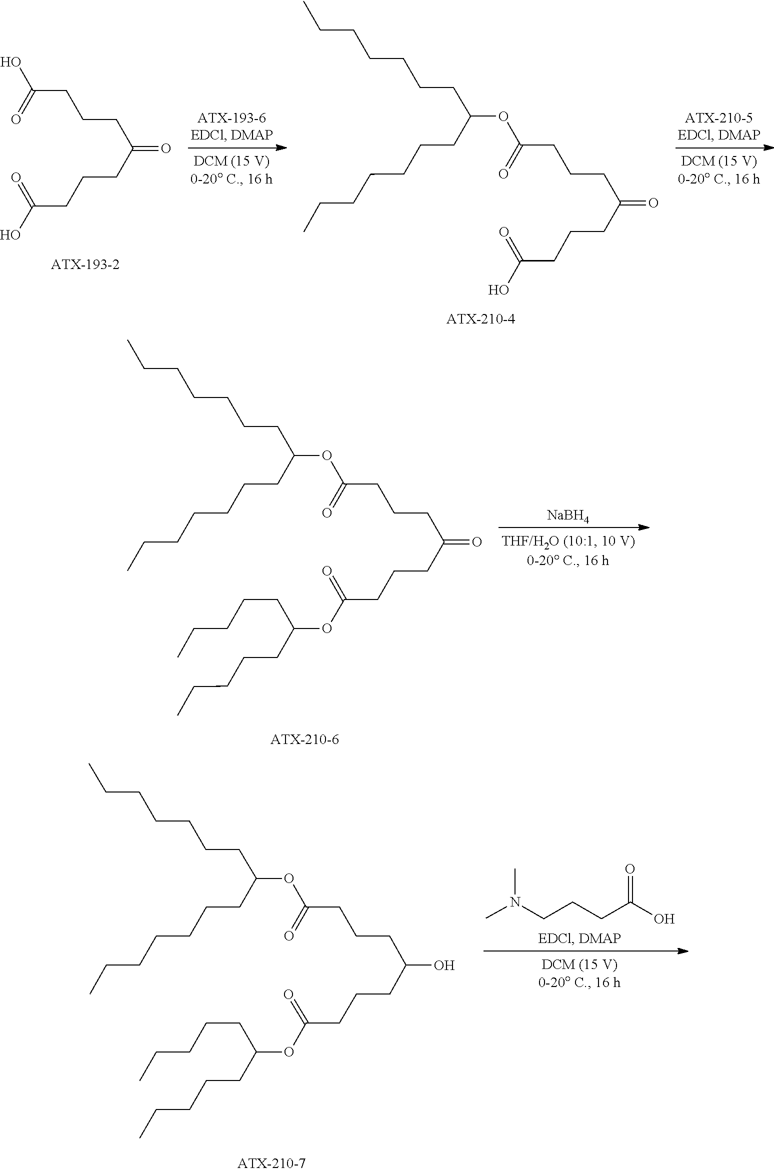

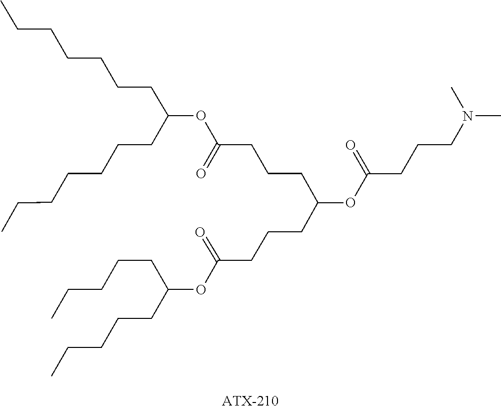

### Synthesis of ATX-210-4

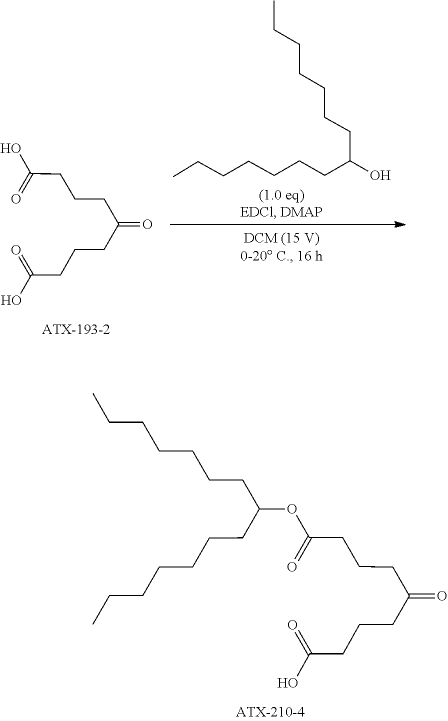 [SMILES: O=C(O)CCCC(=O)CCCC(=O)O ; CCCCCCCC(O)CCCCCCC ; CCCCCCCC(CCCCCCC)OC(=O)CCCC(=O)CCCC(=O)O]

Into a 1-L 3-necked round-bottom flask purged and maintained with an inert atmosphere of nitrogen, was placed 5-oxononanedioic acid (6 g, 1.00 eq), DCM (90 mL). This was followed by the addition of pentadecan-8-ol (6.77 g, 0.0 eq), DMAP (0.72 g, 0.2 eq), to this was added EDCI (6.84 g, 1.2 eq) at 0° C. The resulting solution was stirred for 16 h at room temperature. The reaction was then quenched by the addition of 75 mL of HCl (1 mol/L). The resulting solution was extracted with 2×100 ml of DCM and the organic layers combined. The resulting mixture was washed with 2×100 ml of NaCl. The organic layers were concentrated under vacuum. The product was dissolved in 60 mL DCM and 40 g Silica gel (type: ZCX-2, 100-200 mesh) was added. The mixture was concentrated under vacuum. The residue was applied onto atmospheric silica gel column (400 g, type: ZCX-2, 100-200 mesh) with MeOH/DCM, gradient from 0/1 to 1/10 and collect product eluent (from 1/20-1/10). The collected product phase was concentrated under vacuum. This resulted in 7.2 g (58.8%) of ATX-210-4 as yellow oil. ELSD A:water/0.05% TFA:B:CH₃CN/0.05% TFA 95:5 to 5:95 A/B at 2 min., hold 0.7 min): RT 1.60 min, m/z (Calcd.) 412.32, (found) 435.15 (M+Na).

### Synthesis of ATX-210-5

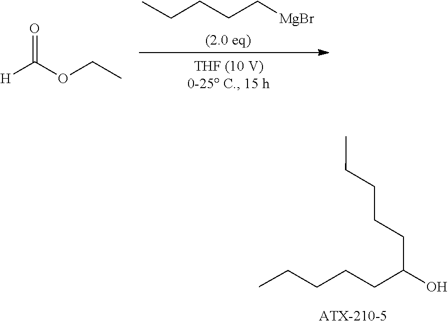

To a 1-L four-neck round-bottle flask with mechanical agitation under N₂, was charged 540 mL of pentylmagnesium bromide (1 mol/L) in THE (200 mL) at 25° C. Charged ethyl formate (20.0 g, 1.0 eq) dropwise with stirring at 0° C. The resulting solution was stirred for 15 h at room temperature. The reaction was then quenched by the addition of 500 mL of NH₄C1. The phases were separated, and the aqueous layer was extracted with 2×500 mL of ethyl acetate. Then combined organic layers; The solvent was dried over anhydrous sodium sulfate. Filtered and concentrated under vacuum. This resulted in 38.9 g (83.6%) of undecan-6-ol as a yellow oil.

### Synthesis of ATX-210-6

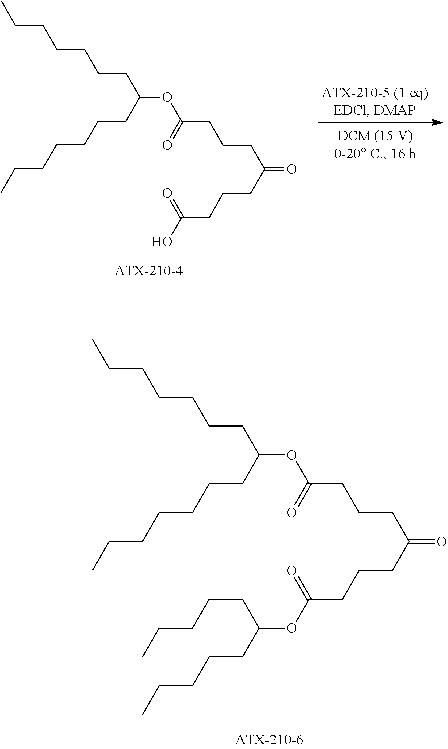 [SMILES: CCCCCCCC(CCCCCCC)OC(=O)CCCC(=O)CCCC(=O)O ; CCCCCCCC(CCCCCCC)OC(=O)CCCC(=O)CCCC(=O)OC(CCCCC)CCCCC]

Into a 1-L 3-necked round-bottom flask purged and maintained with an inert atmosphere of nitrogen, was placed ATX-210-4 (7.2 g, 1.00 eq), DCM (108 mL). This was followed by the addition of undecan-6-ol (3.0 g, 1.0 eq), DMAP (0.43 g, 0.2 eq), to this was added EDCI (4.1 g, 1.2 eq) at 0° C. The resulting solution was stirred for 16 h at room temperature. The reaction was then quenched by the addition of 75 mL of HCl (1 mol/L). The resulting solution was extracted with 2×100 ml of DCM and the organic layers combined. The resulting mixture was washed with 2×100 ml of NaCl. The mixture was dried over anhydrous sodium sulfate and concentrated under vacuum. This resulted in 10 g (99.9%) of ATX-210-6 as a yellow oil and used directly to the next step without further purification. ELSD A:water/0.05% TFA:B:CH₃CN/0.05% TFA 95:5 to 5:95 A/B at 5 min., hold 0.7 min): RT 3.62 min, m/z (Calcd.) 566.49, (found) 589.40 (M+Na).

### Synthesis of ATX-210-7

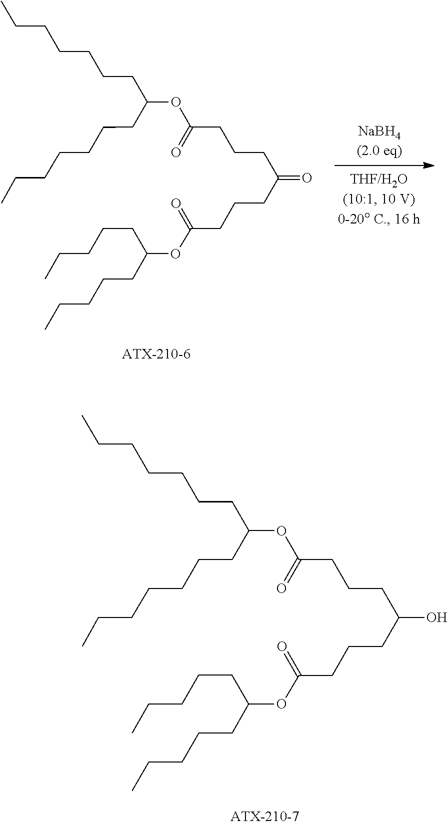 [SMILES: CCCCCCCC(CCCCCCC)OC(=O)CCCC(=O)CCCC(=O)OC(CCCCC)CCCCC ; CCCCCCCC(CCCCCCC)OC(=O)CCCC(O)CCCC(=O)OC(CCCCC)CCCCC]

Into a 250-mL 3-necked round-bottom flask purged and maintained with an inert atmosphere of nitrogen, was placed ATX-210-6 (10 g, 1.0 eq), THF/H₂O (10:1, 100 mL). This was followed by the addition of NaBH₄ (1.34 g, 2.0 eq) at 0° C. The resulting solution was stirred for 16 h at room temperature. The reaction was then quenched by the addition of 100 mL of water/ice. The resulting solution was extracted with 3×100 mL of ethyl acetate and the organic layers combined. The resulting mixture was washed with 2×100 ml of NaCl. The mixture was dried over anhydrous sodium sulfate and the organic layers was concentrated under vacuum. The product was dissolved in 10 mL DCM and 40 g Silica gel (type: ZCX-2, 100-200 mesh) was added. The mixture was concentrated under vacuum. The residue was applied onto atmospheric silica gel column (400 g, type: ZCX-2, 100-200 mesh) with PE/EA, gradient from I/O to 10/1 and collect product eluent (from 20/1-10/1). The collected product phase was concentrated under vacuum. This resulted in 7.1 g (70.7%) of ATX-210-7 as yellow oil and used directly to the next step without further purification. ELSD A:water/0.05% TFA:B:CH₃CN/0.05% TFA 95:5 to 5:95 A/B at 5 min., hold 0.7 min): RT 3.64 min, m/z (Calcd.) 568.50, (found) 591.35 (M+Na).

### Synthesis of ATX-210

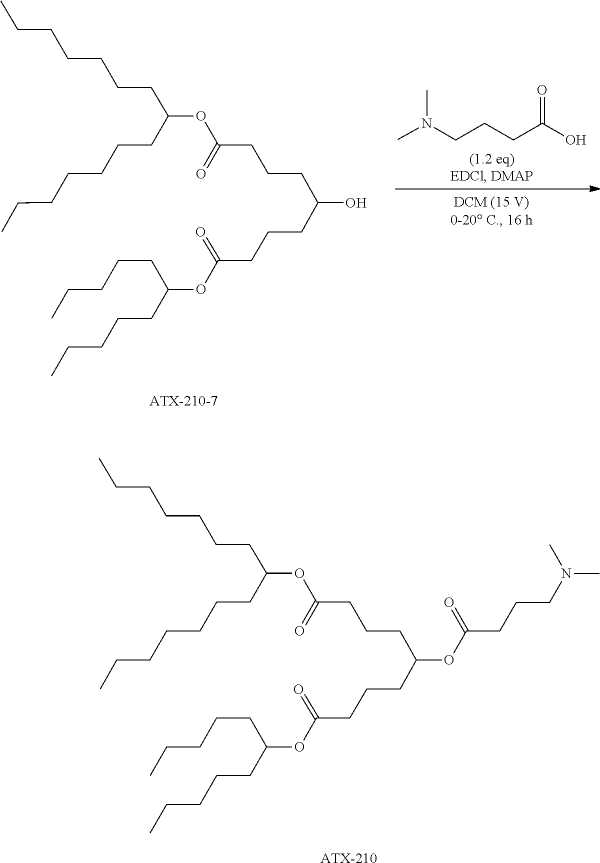

Into a 100-mL 3-necked round-bottom flask purged and maintained with an inert atmosphere of nitrogen, was placed a solution of ATX-210-7 (3.3 g, 1.00 eq) in DCM (50 mL). 4-(dimethylamino)butanoic acid (1.16 g, 1.20 eq), DMAP (0.14 g, 0.20 eq) were added, followed by the addition of EDCI (1.34 g, 1.20 eq) in portions at 0° C. The resulting solution was stirred for 16 h at room temperature. The reaction was then quenched by the addition of 50 mL of NaHCO₃ (1 mol/L). The resulting solution was extracted with 3×50 mL of DCM and the organic layers combined. The resulting mixture was washed with 2×50 mL of brine. The organic layers were concentrated under vacuum. The product was dissolved in 5 mL DCM and 15 g Silica gel (type: ZCX-2, 100-200 mesh) was added. The mixture was concentrated under vacuum. The residue was applied onto atmospheric silica gel column (150 g, type: ZCX-2, 100-200 mesh) with PE/EA, gradient from I/O to 10/1 and collect product eluent (from 20/1-10/1). The collected product phase was concentrated under vacuum. The product was dissolved in heptane (60 mL, 20 V) and the heptane phase was concentrated under vacuum. This resulted in 2.3 g (60.0%) of ATX-210 as yellow oil. ELSD A:water/0.05% TFA:B:CH₃CN/0.05% TFA 95:5 to 5:95 A/B at 2 min., hold 0.7 min): RT 1.84 min, m/z (Calcd.) 681.59, (found) 682.40 (M+H); H-NMR-PH-ARC-LIPID-210-0: (300 MHz, Chloroform-d): δ 4.821-4.904 (3H, m), 2.235-2.357 (8H, m), 2.187-2.204 (6H, s), 1.571-1.831 (16H, m), 1.261 (32H, s), 0.855-0.899 (12H, m).

### Example 7. Synthesis of ATX-230

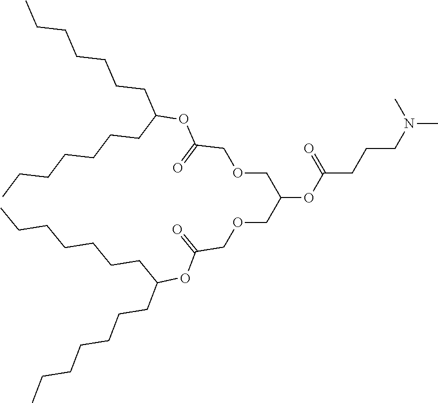 [SMILES: CCCCCCCC(CCCCCCC)OC(=O)COCC(COCC(=O)OC(CCCCCCC)CCCCCCC)OC(=O)CCCN(C)C]

General Scheme:

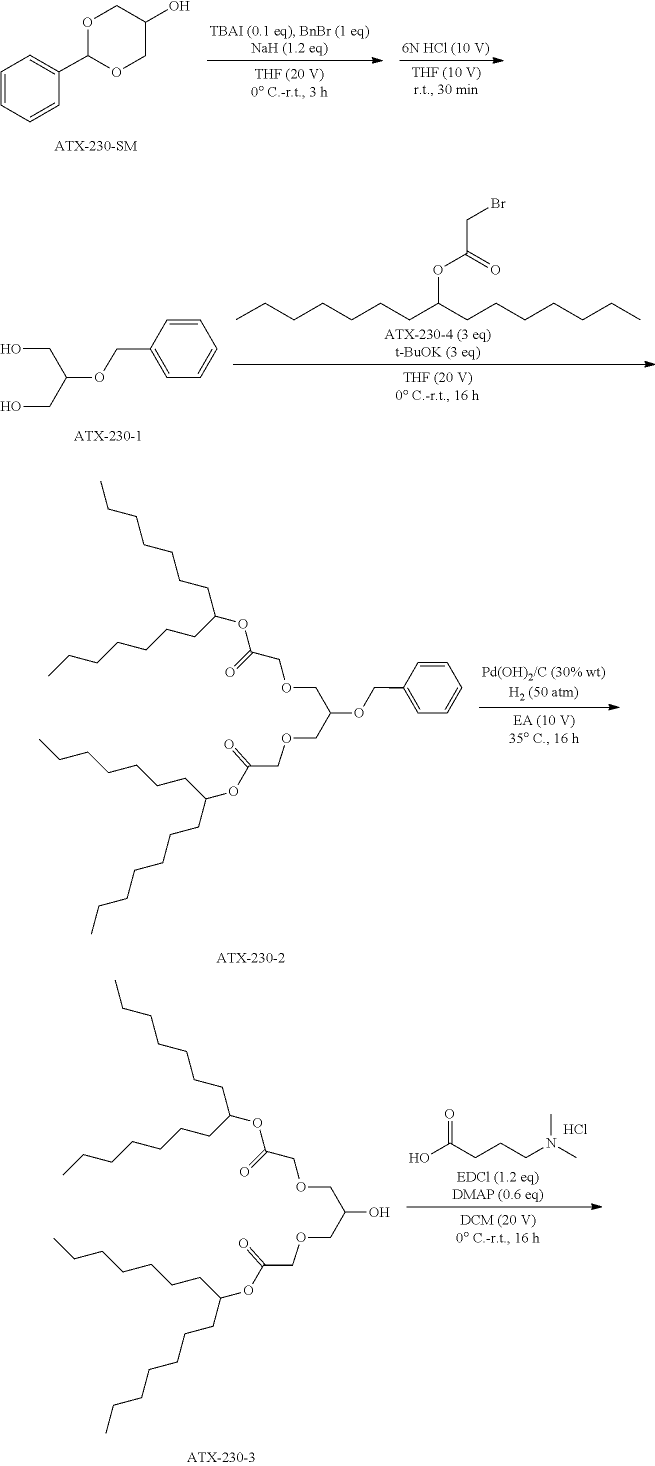

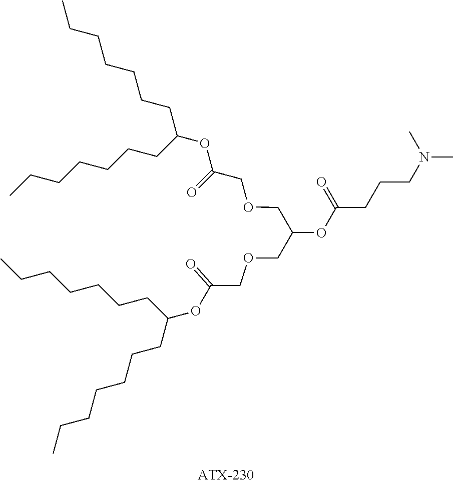

### Synthesis of ATX-230-1

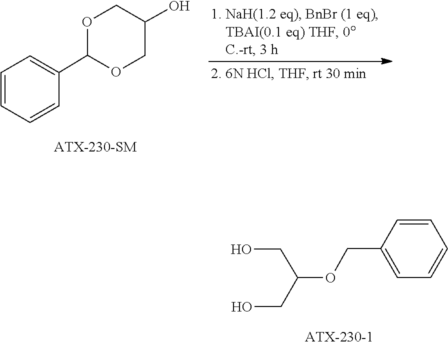 [SMILES: OC1COC(c2ccccc2)OC1 ; OCC(CO)OCc1ccccc1]

Into a 100 mL three-necked round-bottom flask was added ATX-230-SM (2.5 g, 1.0 equiv) in THF (50 mL, 20 V). NaH (560 mg, 60% in mineral oil, 1.2 equiv) was added to the reaction mixture at 0° C. in several portions and stirred for 30 min. Benzyl bromide (2.4 g, 1.0 equiv) and tetra-n-butyl ammonium iodide (TBAI) (1.5 g, 0.1 equiv) were added to the reaction mixture at 0° C. The resulting solution was stirred for 2 h at room temperature, HPLC indicated complete consumption of ATX-230-SM. The reaction was quenched by adding ice-water to the system carefully and stirred for 10 min. The organic solvent was evaporated in vacuum and the aqueous phase was extracted with DCM (2×25 mL, 20 V). Concentrated the organic solvent under vacuum. The residue was dissolved in THF (25 mL, 10 V), and added 6 mol/L aqueous HCl (25 mL, 10 V) at room temperature. The resulting solution was stirred for 30 min at room temperature. The pH value of the solution was adjusted to 7-8 with aqueous NaHCO₃ solution. The resulting solution was extracted with ethyl ether (2×25 mL, 20 V). The organic layers were combined, dried with anhydrous MgSO₄ and then filtered. To the filtrate was charge 8 g of silica gel (type: ZCX-2, 100-200 mesh, 3.20 w./w.), concentrate to no fraction under vacuum while maintaining the temperature below 20° C. Charge 40 g of silica gel (type: ZCX-2, 100-200 mesh, 16.00 w./w.) to the column, followed by the last step prepared dry silica gel which absorbed the reaction mixture. Using combi-flash to purify the product. Elute with PE/EA (volume ratio, gradient from 100/0 to 95:5). Concentrated product fraction under vacuum to afford the ATX-230-1 (1.5 g, 60% yield) as a white solid. ELSD A:water/0.05% TFA:B:CH₃CN/0.05% TFA 95:5 to 5:95 A/B at 2 min., hold 0.7 min): RT 0.79 min, m/z (Calcd.) 182.09, (found) 205.10 (M+Na); H-NMR-PH-ARC-LIPID-230-1: (300 MHz, Chloroform-d): δ 7.40-7.29 (5H, m), 4.66 (2H, s), 3.83-3.71 (4H, m), 3.64-3.58 (1H, m).

### Synthesis of ATX-230-2

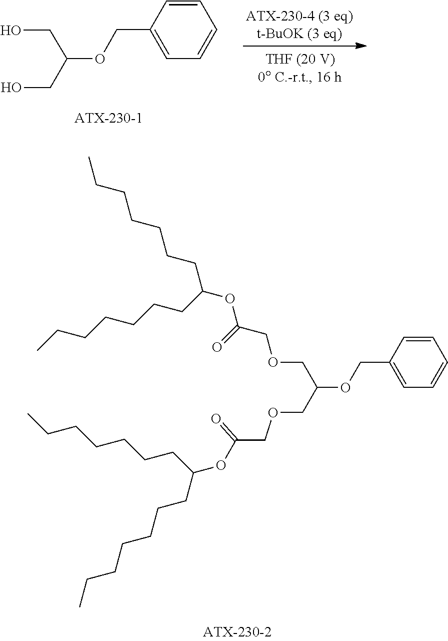 [SMILES: OCC(CO)OCc1ccccc1 ; CCCCCCCC(CCCCCCC)OC(=O)COCC(COCC(=O)OC(CCCCCCC)CCCCCCC)OCc1ccccc1]

Step 1: To a solution of pentadecane-8-ol (150.0 g, 1.0 equiv) in DCM (3 L, 20 V) in dry three-necked flask with N₂, and then TEA (266.0 g, 4.0 equiv) was added in one portion, followed by the addition of bromoacetylbromide (526.0 g, 4.0 equiv) at 0° C. The reaction was stirred for 3 days at room temperature and quenched by the addition of a saturated aqueous NH₄Cl solution (10 L, 66.7 V) at 0° C. The crude compound was extracted DCM (10 L*3, 200 V). The combined organic fractions were washed with brine (10 L, 66.7 V) and dried over anhydrous MgSO₄ and filtered. To the filtrate was added 500 g of silica gel (type: ZCX-2, 100-200 mesh, 3.33 w./w.), concentrate to no fraction under vacuum while maintaining the temperature below 35° C. Charge 2.5 kg of silica gel (type: ZCX-2, 100-200 mesh, 16.67 w./w.) to the column, followed by the last step prepared dry silica gel which absorbed the reaction mixture. Using combi-flash to purify the product. Elute with PE/EA (volume ratio). (gradient at 100:0 collect every 3±0.5 L). Take sample for TLC (PE:EA=8:1, Rf=0.2) analysis. Combine qualified fractions and concentrated to dry. ELSD A:water/0.05% TFA:B:CH₃CN/0.05% TFA 80:20 to 20:80 A/B at 3 min., hold 0.98 min): RT 0.98 min, m/z (Calcd.) 348.17, (found) 390.30 (M+Na+H₂O); H-NMR-PH-ARC-LIPID-230-4: (300 MHz, Chloroform-d): δ 5.01-4.87 (1H, m), 3.81 (2H, s), 1.57 (4H, m), 1.34 (22H, m), 0.88 (6H, t).

Step 2: Into a 100 mL three-necked round-bottom flask was added ATX-230-1 (1.5 g, 1.0 equiv) in THE (30 mL, 20 V). t-BuOK (1.38 g, 1.5 equiv) was added to the reaction mixture at 0° C. with portions and stirred for 30 min. ATX-230-4 (4.3 g, 1.5 equiv) was added to the reaction mixture at 0° C. with portions. The resulting solution was stirred for 16 h at room temperature. Additional t-BuOK (1.38 g, 1.5 equiv) and ATX-230-4 (4.3 g, 1.5 equiv) was added to the reaction mixture at room temperature. The resulting solution was stirred for 16 h at room temperature. LCMS indicated complete consumption of ATX-230-1. The reaction was then quenched by the addition of ammonium chloride solution (15 mL, 10 V). The resulting solution was extracted with Et₂O (2*30 mL, 40 V). The organic layers were combined, dried with anhydrous MgSO₄ and then filtered. To the filtrate was charged 3 g of silica gel (type: ZCX-2, 100-200 mesh, 2.00 w./w.), concentrated under vacuum while maintaining the temperature below 20° C. to adsorb the compound. The material was purified on a 20 g of Combi-flash silica gel column using PE/EA (volume ratio, gradient from 100/0 to 95:5) to elute the product. Fractions were pooled and concentrated under vacuum to afford the ATX-230-2 (2.1 g, 35.5% yield) as a yellow solid. ELSD A:water/0.05% TFA:B:CH₃CN/0.05% TFA 80:20 to 20:80 A/B at 3 min., hold 2.6 min): RT 2.62 min, m/z (Calcd.) 718.57, (found) 741.50 (M+Na); H-NMR-PH-ARC-LIPID-230-2: (300 MHz, Chloroform-d): δ 7.39-7.27 (5H, m), 4.99-4.93 (2H, m), 4.52 (2H, s), 4.10 (4H, s), 3.99-3.68 (m, 5H), 1.53 (9H, m), 1.49 (42H, m), 1.26-1.24 (12H, t).

### Synthesis of ATX-230-3

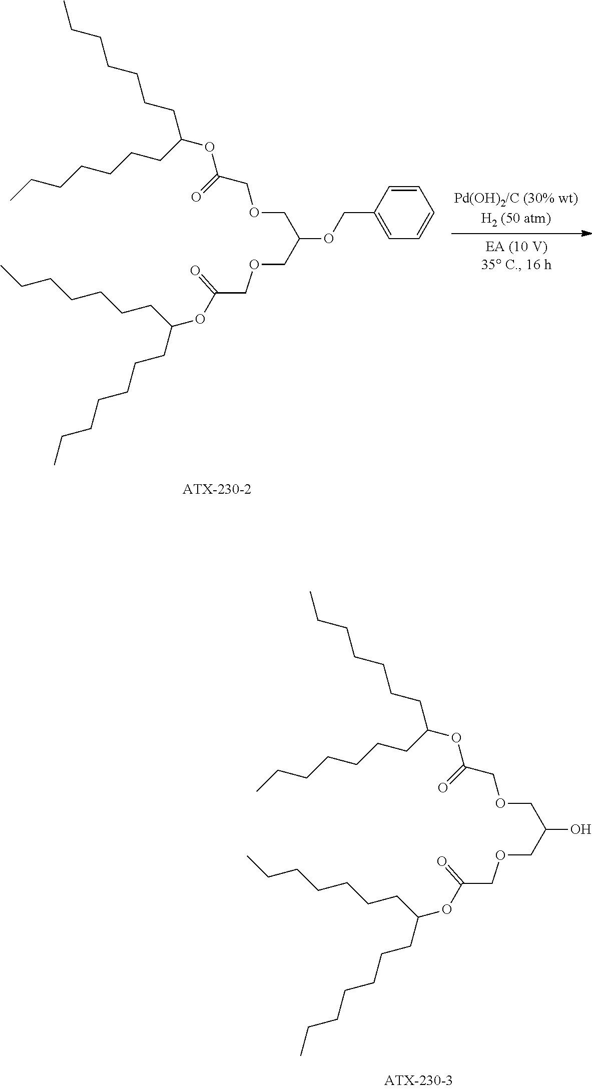 [SMILES: CCCCCCCC(CCCCCCC)OC(=O)COCC(COCC(=O)OC(CCCCCCC)CCCCCCC)OCc1ccccc1 ; CCCCCCCC(CCCCCCC)OC(=O)COCC(O)COCC(=O)OC(CCCCCCC)CCCCCCC]

Charge ATX-230-2 (2.1 g, 1.0 equiv) and 20% Pd(OH)₂/C (0.63 g, 30% wt) in EA (21 mL, 10 V) into autoclave at room temperature. Stirred for 16 h at 35° C. under the hydrogen atmosphere (50 atm). TLC showed that ATX-230-2 was completely converted. The reaction mixture was filtered and concentrated under vacuum at 40° C. to get ATX-230-3 (1.7 g, 95% yield) as a white solid. ELSD A:water/0.05% TFA:B:CH₃CN/0.05% TFA 80:20 to 20:80 A/B at 3 min., hold 2.6 min): RT 1.90 min, m/z (Calcd.) 628.53, (found) 651.50 (M+Na); H-NMR-PH-ARC-LIPID-230-3: (300 MHz, Chloroform-d): δ 4.99-4.91 (2H, dd), 4.03 (4H, s), 3.67-3.37 (4H, m), 1.56-1.49 (9H, m), 1.29-1.25 (40H, m), 0.97-0.85 (12H, t).

### Synthesis of ATX-230

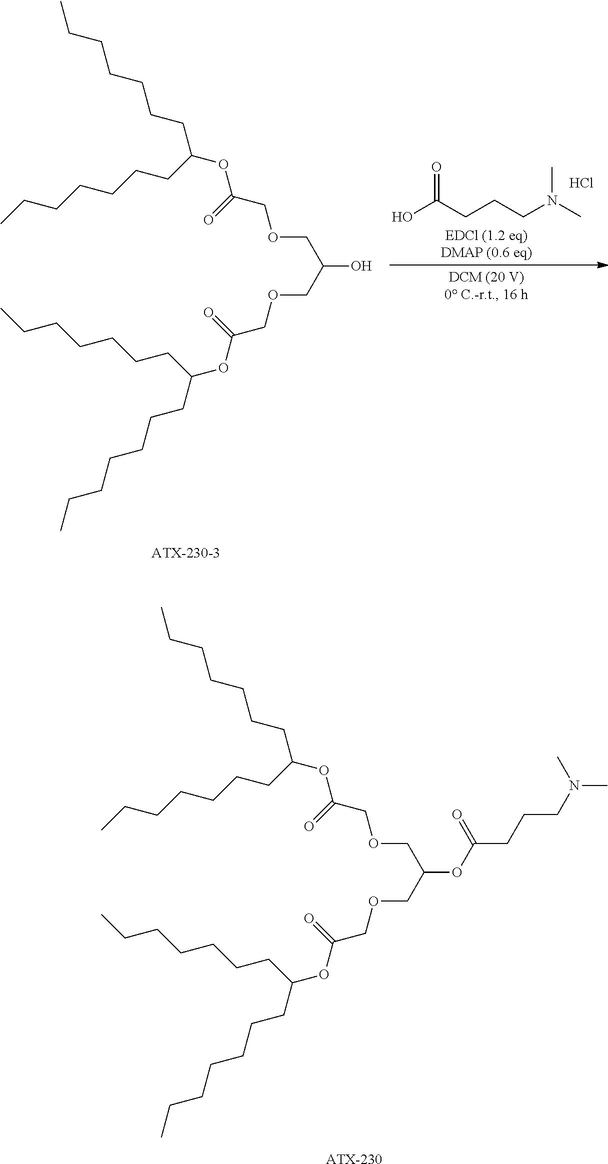

To a 100 mL three-necked round-bottom flask was added ATX-230-3 (1.7 g, 1.0 equiv), 4 (dimethylamino)butanoic acid hydrochloride (450 mg, 1.0 equiv) and DMAP (198 mg, 0.6 equiv) in DCM (34 mL, 20 V). EDCI (620 mg, 1.2 equiv) was added to the reaction mixture at 0° C. in several portions. The resulting solution was stirred for 16 h at 20° C. The reaction system was quenched with 10% aqueous citric acid solution (17 mL, 10 V) and the organic phase was collected. The organic solution was washed with 10% aqueous citric acid solution (17 mL, 10 V) followed by brine (17 mL, 10 V). The organic phase was dried with anhydrous MgSO₄ and filtered. The mixture was adsorbed on 5 g of silica gel (type: ZCX-2, 100-200 mesh, 2.94 w/w) and purified on combi-flash silica gel column (40 g) by eluting with DCM/MeOH gradient from 100:0 to 98:2. Product containing fractions were pooled and concentrated under vacuum to afford 1.2 g (65% yield) ATX-230 as a light-yellow oil. ELSD A:water/0.05% TFA:B:CH₃CN/0.05% TFA 80:20 to 20:80 A/B at 3 min., hold 2.6 min): RT 0.96 min, m/z (Calcd.) 741.62, (found) 742.6 [M+1]⁺; H-NMR-PH-ARC-ATX-230-0: (400 MHz, CDCl₃, ppm) δ 5.181 (quint, J=5.0 Hz, 1H), 4.931 (quint, J=6.3 Hz, 2H), 4.081 (s, 4H), 3.830-3.700 (m, 4H), 2.341 (dt, J=41.4, 7.4 Hz, 4H), 2.212 (s, 6H), 1.800 (quint, J=7.4 Hz, 2H), 1.530 (d, J=3.9 Hz, 8H), 1.25 (m, 40H), 0.900-0.830 (m, 12H).

### Example 8. Synthesis of ATX-231

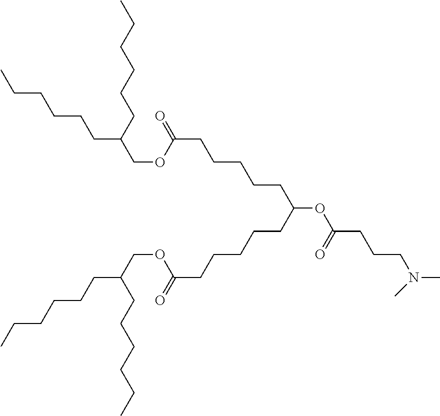 [SMILES: CCCCCCC(CCCCCC)COC(=O)CCCCCC(CCCCCC(=O)OCC(CCCCCC)CCCCCC)OC(=O)CCCN(C)C]

General Scheme:

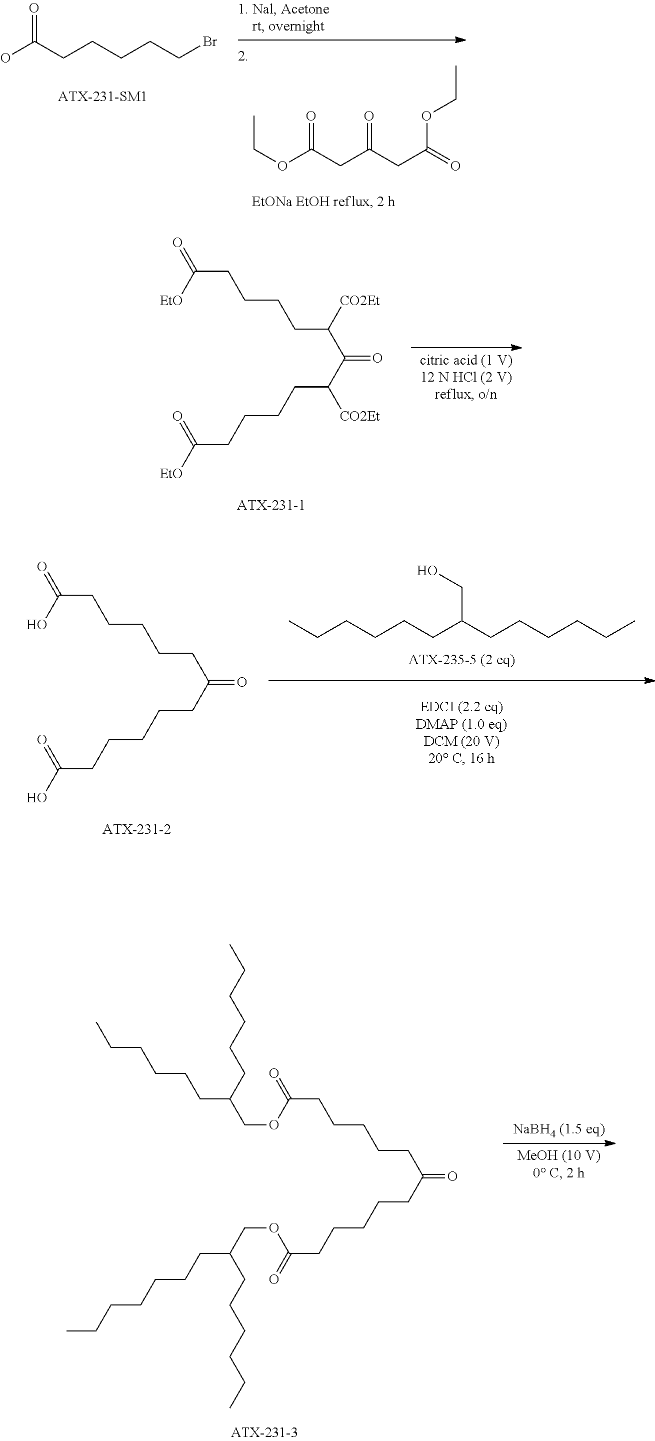 [SMILES: O=C(O)CCCCCBr ; CCOC(=O)CC(=O)CC(=O)OCC ; CCOC(=O)CCCCC(C(=O)OCC)C(=O)C(CCCCC(=O)OCC)C(=O)OCC ; O=C(O)CCCCCC(=O)CCCCCC(=O)O ; CCCCCCC(CO)CCCCCC ; CCCCCCCC(CCCCCC)COC(=O)CCCCCC(=O)CCCCCC(=O)OCC(CCCCCC)CCCCCC]

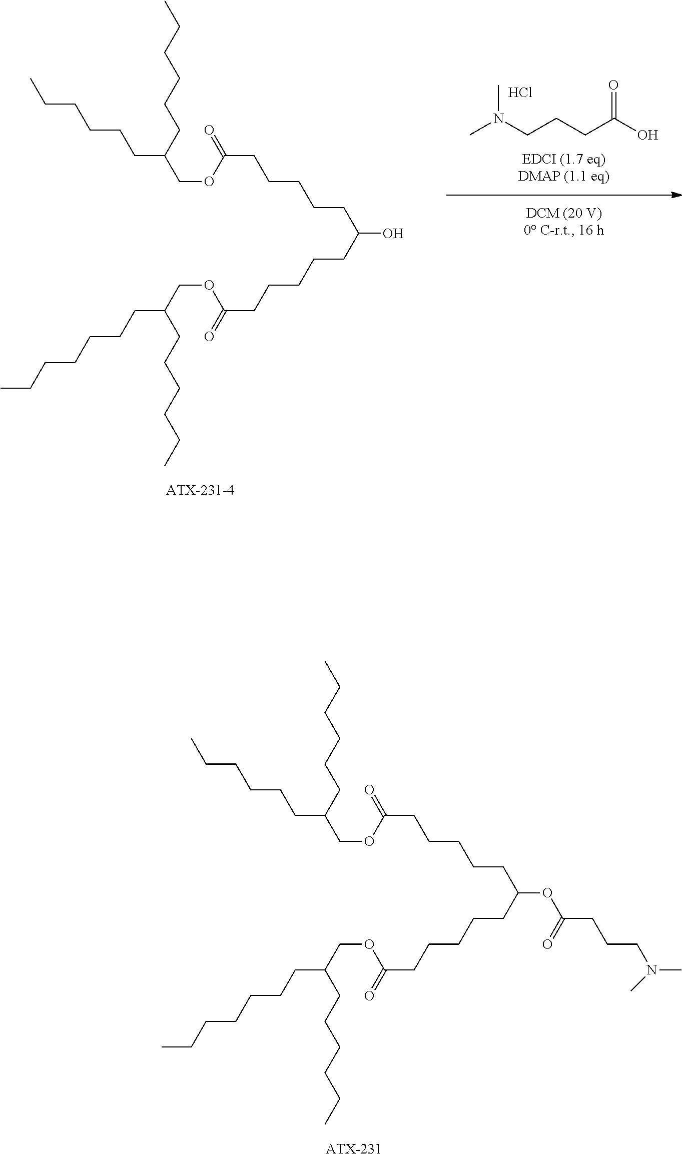

### Synthesis of ATX-231-1

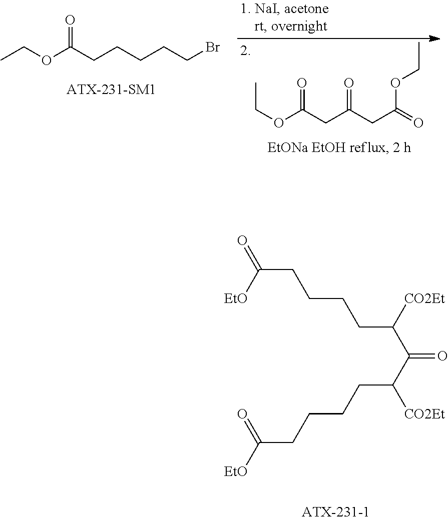 [SMILES: CCOC(=O)CCCCCBr ; CCOC(=O)CC(=O)CC(=O)OCC ; CCOC(=O)CCCCC(C(=O)OCC)C(=O)C(CCCCC(=O)OCC)C(=O)OCC]

To a 1 L three-necked round-bottom flask was added ethyl ATX-231-SM1 (50.0 g, 1.0 equiv) and sodium iodide (180 g, 4.4 equiv) in acetone (500 mL, 10 V). The reaction was stirred at room temperature for overnight. The reaction mixture was diluted with water (400 mL, 8 V) and extracted with diethyl ether (400 mL, 8 V). The organic fraction was washed with water, dried over anhydrous magnesium sulfate, filtered and removed the solvent. Sodium ethoxide (10.8 g, 2.1 equiv) was dissolved in absolute ethanol (90 mL, 2 V). Diethylacetone dicarboxylate (36.0 g, 1.12 equiv) was added and the solution heated to reflux. Then ethyl 6-iodocaproate (24.0 g, 1.0 equiv) was added slowly and the solution refluxed for an hour. A solution of sodium ethoxide (10.8 g, 2.1 equiv) in ethanol (90 mL, 2 V) was added, followed by ethyl 6-iodovalerate (24.0 g, 1.0 equiv). The solution was refluxed overnight. The reaction mixture was cooled, diluted with water (400 mL, 8 V) and extracted with diethyl ether (400 mL, 8 V). Concentrated under vacuum to afford 47.5 g (crude) ATX-231-1 as yellow oil.

### Synthesis of ATX-231-2

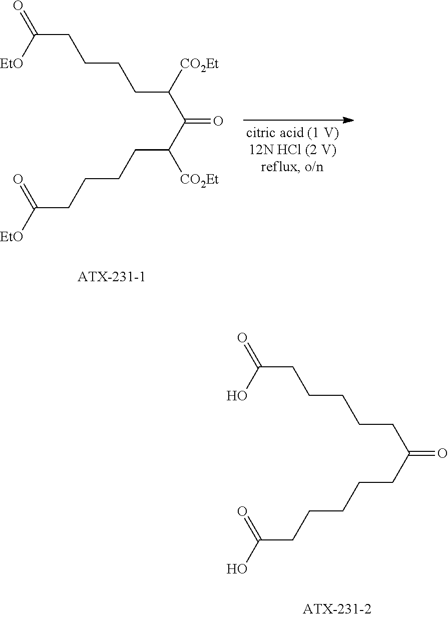 [SMILES: CCOC(=O)CCCCC(C(=O)OCC)C(=O)C(CCCCC(=O)OCC)C(=O)OCC ; O=C(O)CCCCCC(=O)CCCCCC(=O)O]

To a 100 mL three-necked round-bottom flask was added ATX-231-1 (40.0 g, 1.0 equiv) in citric acid (40 mL, 1 V) and HCl (80 mL, 2V, 12 mol/L). The reaction solution was refluxed for overnight. The solution was cooled, diluted with water, and extracted with dichloromethane. The solvent was removed and the residue was recrystallized from acetone and dried under vacuum to get 4 g (14%) ATX-231-2 as a white solid. ELSD A:water/5 mM NH₄HCO₃:B:CH₃CN 80:20 to 90:10 A/B at 2 min.): RT 0.16 min, m/z (Calcd.) 258.15, (found) 257.30 [M+1]+; H-NMR-PH-ARC-ATX-231-1: (400 MHz, CDCl₃, ppm) δ 2.5-2.49 (m, 2H), 2.42-2.32 (m, 2H), 2.19-2.15 (m, 4H), 2.00-1.98 (m, 8H), 1.51-1.47 (m, 4H).

### Synthesis of ATX-231-3

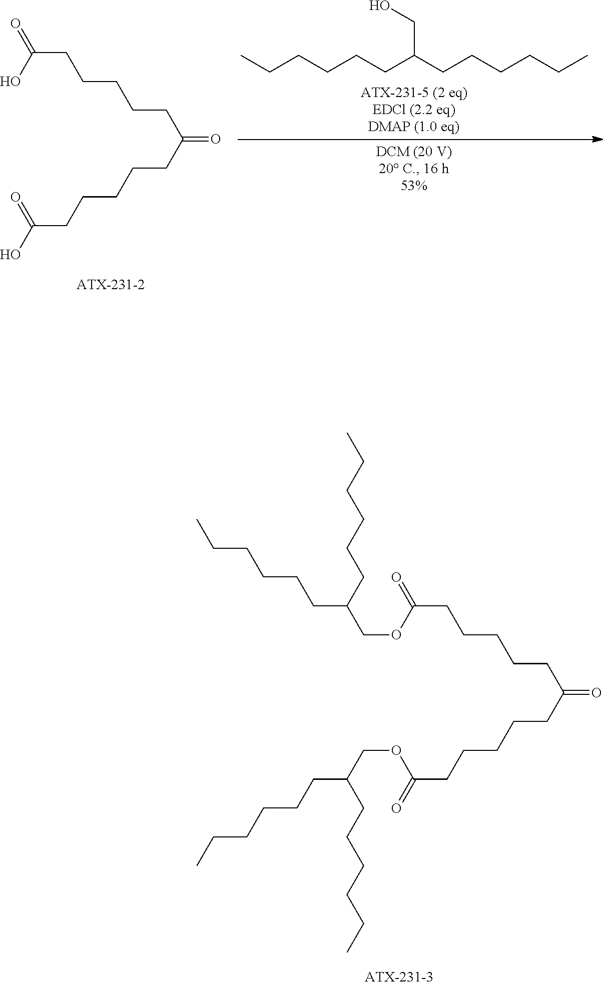 [SMILES: O=C(O)CCCCCC(=O)CCCCCC(=O)O ; CCCCCCC(CO)CCCCCC ; CCCCCCC(CCCCCC)COC(=O)CCCCCC(=O)CCCCCC(=O)OCC(CCCCCC)CCCCCC]

Step 1:

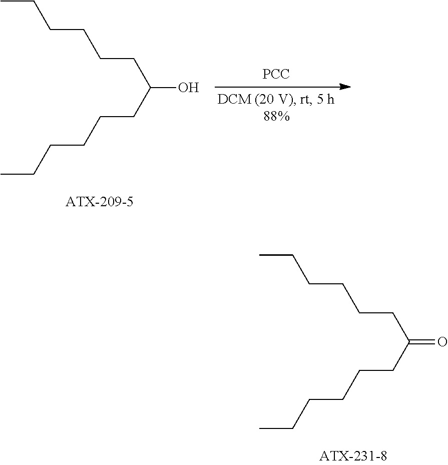

Added DCM (300 ml, 20 V), ATX-209-5 (15 g, 1 eq) and pyridinium chlorochromate (PCC, 40 g, 2.5 eq) to a 500 ml three-neck flask. The resulting solution was stirred for 5 h at room temperature. TLC observation indicated complete conversion of ATX-209-5. The solvent was removed by distillation under vacuum. The crude product was applied onto a silica gel column and the product was eluted with ethyl acetate/petroleum ether (1:10) gradient to get the ATX-231-8 (13 g, 88% yield) as colorless clear oil. ¹H NMR (300 MHz, DMSO-d6) δ 2.38 (t, J=7.3 Hz, 4H), 1.53-1.36 (m, 4H), 1.34-1.15 (m, 12H), 0.89-0.80 (m, 6H).

Step 2:

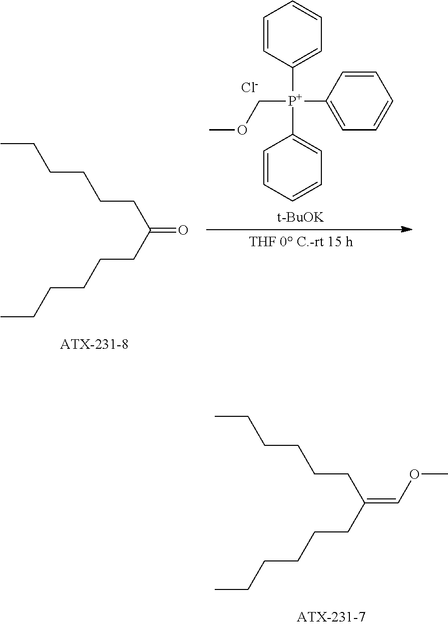 [SMILES: "t-BuOK" ; CCCCCCC(=O)CCCCCC ; COC[P+](c1ccccc1)(c1ccccc1)c1ccccc1 ; [Cl-] ; CCCCCCC(=COC)CCCCCC]

Added THE (260 ml, 20 V) and (methoxymethy)triphenylphosphonium chloride (32 g, 1.6 eq) to a 500 ml three-neck flask followed by t-BuOK (11.8, 1.6 eq) to the mixture in batches at 0° C. Stirred at 0° C. for 1 h. Added ATX-231-8 (13 g, 1 eq) to the reaction mixture. Stirred at room temperature for 15 h. Added Ammonium chloride aqueous solution (10 wt %, 260 ml, 20 V) to the system to quench. Added MTBE (260 ml, 20 V) and extracted to the reaction mixture and collected organic phase. After concentration of the organic phase, the mixture was applied onto a silica gel column with ethyl acetate/petroleum ether (2:98). Got the ATX-231-7 (10 g, 70% yield) as oil. ¹H NMR (300 MHz, Chloroform-d) δ 5.74 (s, 1H), 3.51 (s, 3H), 2.03 (t, J=7.3 Hz, 2H), 1.88-1.81 (m, 2H), 1.42-1.20 (m, 16H), 0.93-0.83 (m, 6H).

Step 3:

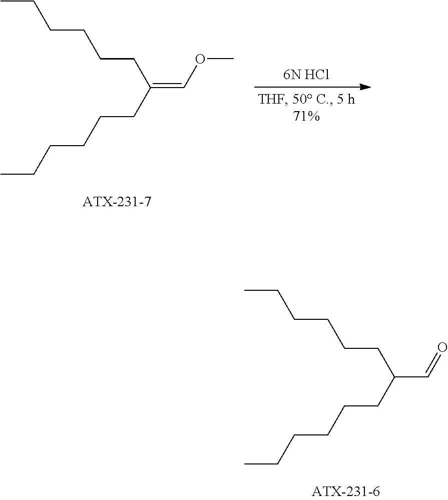

Added THF (50 ml, 5 V), ATX-231-7 (10 g, 1 eq) and 6N HCl (20 ml, 2 V) to a 250 ml three-neck flask at room temperature. Stirred at 50° C. for 5 h. Added 3N NaOH (40 ml, 4 V) and MTBE (100 ml, 10 V) to the reaction mixture and the product was extracted into the ether phase. Collected ether phase and concentrated under vacuum to get the ATX-231-6 (6.57 g, 71% yield) as an oil. ¹H NMR (300 MHz, Chloroform-d) δ 9.49 (d, J=3.1 Hz, 1H), 3.62 (m, 1H), 1.22 (m, 20H), 0.88-0.78 (m, 6H).

Step 4:

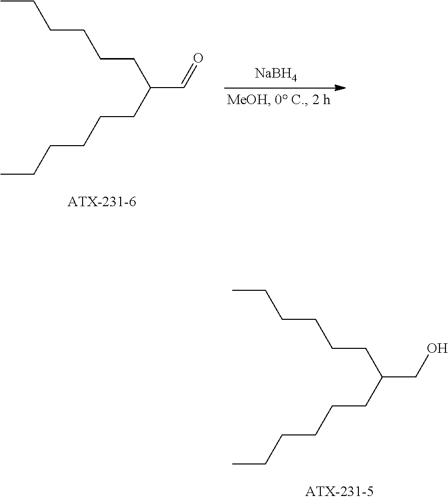

Added MeOH (65 ml, 10 V) and ATX-231-6 (6.57 g, 1 eq) to a 100 ml three-neck flask at room temperature. Added NaBH₄ (1.76, 1.5 eq) in batches to the reaction mixture at 0° C. and stirred at 0° C. for 2 h. Added citric acid solution (10 wt %, 65.7 ml, 10 V) to the reaction mixture at 0° C. The product was extracted into methyl tert-butyl ether (MTBE, 65 ml, 10 V), organic phase was collected and concentrated under vacuum to get the ATX-231-5 (5.2 g, 79% yield) as an oil. ¹H NMR (300 MHz, Chloroform-d) δ 3.53 (d, J=5.4 Hz, 2H), 3.48 (s, 1H), 1.28 (m, 20H), 0.93-0.83 (m, 6H).

Step 5: To a 250 mL three-necked round-bottom flask was added ATX-231-2 (3.0 g, 1.0 equiv), ATX-231-5 (4.97 g, 2.0 equiv) and DMAP (1.42 g, 1.0 equiv) in DCM (60 mL, 20 V). Then, EDCI (4.9 g, 2.2 equiv) was added to the reaction mixture at 0° C. in several portions. The resulting solution was stirred for 16 h at 20° C., TLC indicated complete consumption of ATX-231-2. The reaction was quenched with 10% aqueous citric acid solution (30 mL, 10 V). The isolated organic phase was washed once more with 10% aqueous citric acid solution (30 mL, 10 V) followed by brine (30 mL, 10 V). The organic phase was dried with anhydrous MgSO₄ and then filtered. The crude product was adsorbed on 6 g of silica gel (type: ZCX-2, 100-200 mesh, 2.00 w./w), and purified on a 30 g of silica gel column, using petroleum ether/ethyl acetate gradient from 100:0 to 98:2. Qualified fractions, post TLC analysis (10:1 PE:EA), were pooled and concentrated to dryness under vacuum to afford 4 g (53% yield) ATX-231-3 as a colorless oil. ELSD A:water/0.05% TFA:B:CH₃CN/0.05% TFA 80:20 to 20:80 A/B at 3.5 min): RT 2.89 min, m/z (Calcd.) 650.58, (found) 673.50 (M+Na); H-NMR-PH-ARC-LIPID-230-2: (300 MHz, Chloroform-d): δ 3.97-3.96 (d, J=2.4 Hz, 4H), 2.45-2.43 (m, 4H), 2.38-2.28 (m, 4H), 1.66-1.60 (9H, m), 1.49 (48H, m), 0.86-0.88 (12H, t).

### Synthesis of ATX-231-4

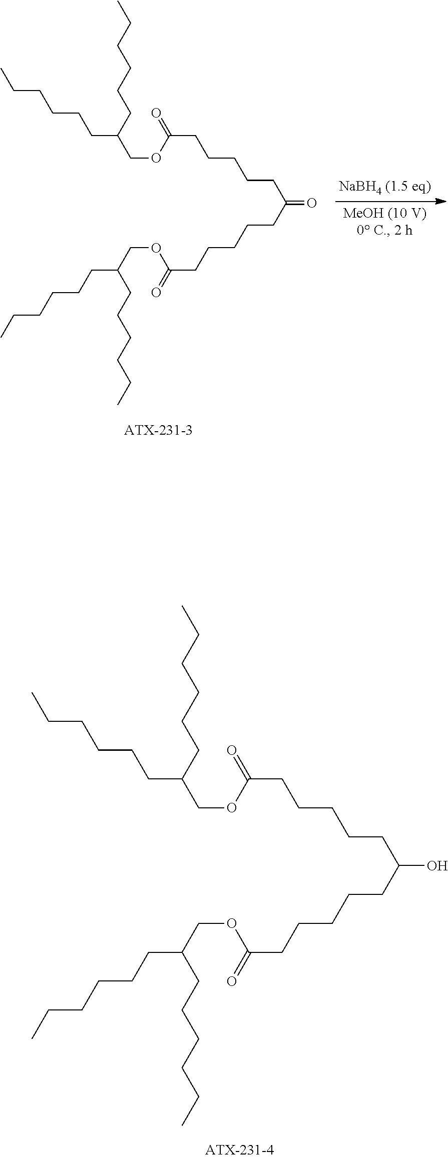 [SMILES: CCCCCCC(CCCCCC)COC(=O)CCCCCC(=O)CCCCCC(=O)OCC(CCCCCC)CCCCCC ; CCCCCCC(CCCCCC)COC(=O)CCCCCC(O)CCCCCC(=O)OCC(CCCCCC)CCCCCC]

To a 100 mL three-necked flask was added ATX-231-3 (4.0 g, 1 equiv) in MeOH (40 mL, 10 V) at room temperature. Then, NaBH₄ (0.34 g, 1.5 equiv) was added to the reaction mixture at 0° C. in several portions. The resulting solution was stirred for 2 h at 0° C. TLC analysis indicated complete consumption of ATX-231-3. The reaction was quenched by the addition of water (40 mL, 10 V). The product was extracted with MTBE twice (2×20 ml, 10 V). The organic phase was dried with anhydrous MgSO₄, filtered and concentrated to dryness under vacuum to afford 3.4 g (85% yield) ATX-231-4 as colorless oil. ELSD A:water/0.05% TFA:B:CH₃CN/0.05% TFA 80:20 to 20:80 A/B at 3.5 min): RT 3.03 min, m/z (Calcd.) 652.60, (found) 675.50 (M+Na); H-NMR-PH-ARC-LIPID-230-2: (300 MHz, Chloroform-d): δ 4.00-3.98 (d, J=7.6 Hz, 4H), 3.59-3.51 (m, 1H), 2.35-2.30 (m, 4H), 1.68-1.63 (m, 6H), 1.55-1.29 (m, 55H), 0.92-0.88 (12H, t).

### Synthesis of ATX-231

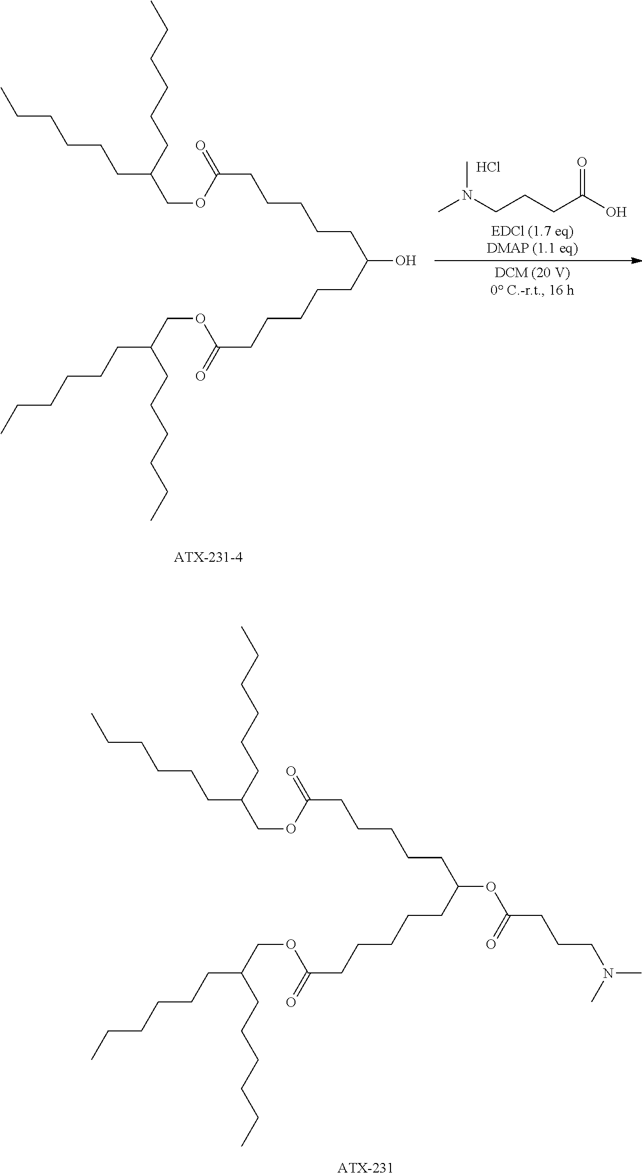

To a 100 mL three-necked round-bottom flask was added ATX-231-4 (2.0 g, 1.0 equiv), 4-(dimethyl-amino)butanoic acid hydrochloride (0.81 g, 1.6 equiv) and DMAP (0.4 g, 1.1 equiv) in DCM (60 mL, 30 V). Then, EDCI (1.0 g, 1.7 equiv) was added to the reaction mixture at 0° C. in several portions. The resulting solution was stirred for 16 h at room temperature, TLC indicated complete consumption of ATX-231-4. The reaction was quenched with 10% aqueous citric acid solution (20 mL, 10 V) and organic phase was isolated. The organic phase was washed with 10% additional aqueous citric acid solution (20 mL, 10 V) followed by brine (20 mL, 10 V), dried with anhydrous MgSO₄ and filtered. The crude product was adsorbed on 6 g of silica gel (type: ZCX-2, 100-200 mesh, 3.00 w./w.) and purified on a Combi-flash system using a 30 g of silica gel column. The product was eluted with a gradient of 100:0 to 98:2 petroleum ether ethyl acetate. Fractions were analyzed (TLC, EA:PE=1:10), pooled concentrated to dryness under vacuum to afford 1.5 g (75% yield) ATX-231 as light-yellow oil. ELSD A:water /0.05% TFA:B:CH₃CN/0.05% TFA 80:20 to 20:80 A/B at 3.5 min): RT 1.90 min, m/z (Calcd.) 766.25, (found) 767.23 (M+H). ¹H-NMR-PH-ARC-ATX-231-0: (300 MHz, CDCl₃, ppm) δ 4.892-4.851 (m, 1H), 3.988-3.969 (d, J=5.8 Hz, 4H), 2.957-2.905 (t, J=8.2 Hz, 2H), 2.713 (s, 6H), 2.445 (t, J=6.8 Hz, 2H), 2.308 (t, J=7.4 Hz, 4H), 2.117 (quint, J=6.9 Hz, 2H), 1.650-1.521 (m, 10H), 1.288 (bs, 48H), 0.921-0.900 (m, 12H).

### Example 9. Synthesis of ATX-232

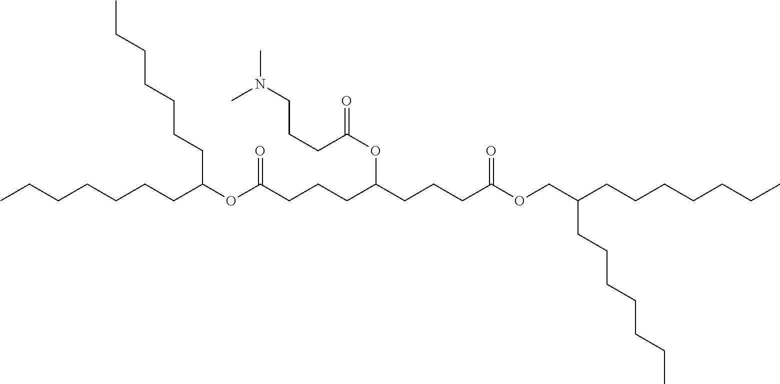 [SMILES: CCCCCCCC(CCCCCCC)COC(=O)CCCC(CCCC(=O)OC(CCCCCCC)CCCCCCC)OC(=O)CCCN(C)C]

General Scheme:

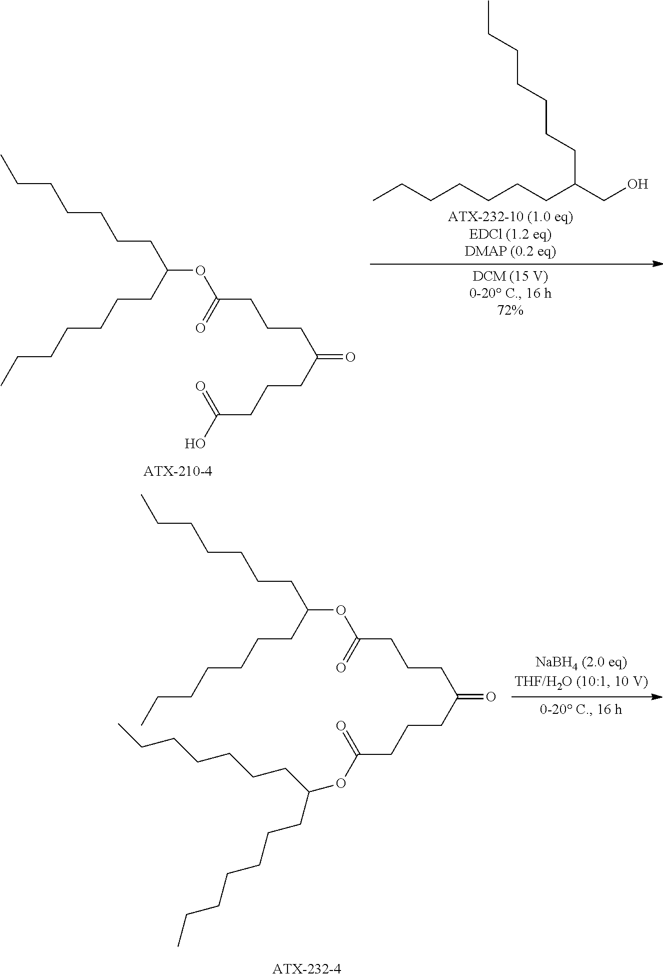 [SMILES: CCCCCCCC(CCCCCCC)OC(=O)CCCC(=O)CCCC(=O)O ; CCCCCCCC(CO)CCCCCCC ; CCCCCCCC(CCCCCCC)OC(=O)CCCC(=O)CCCC(=O)OC(CCCCCCC)CCCCCCC]

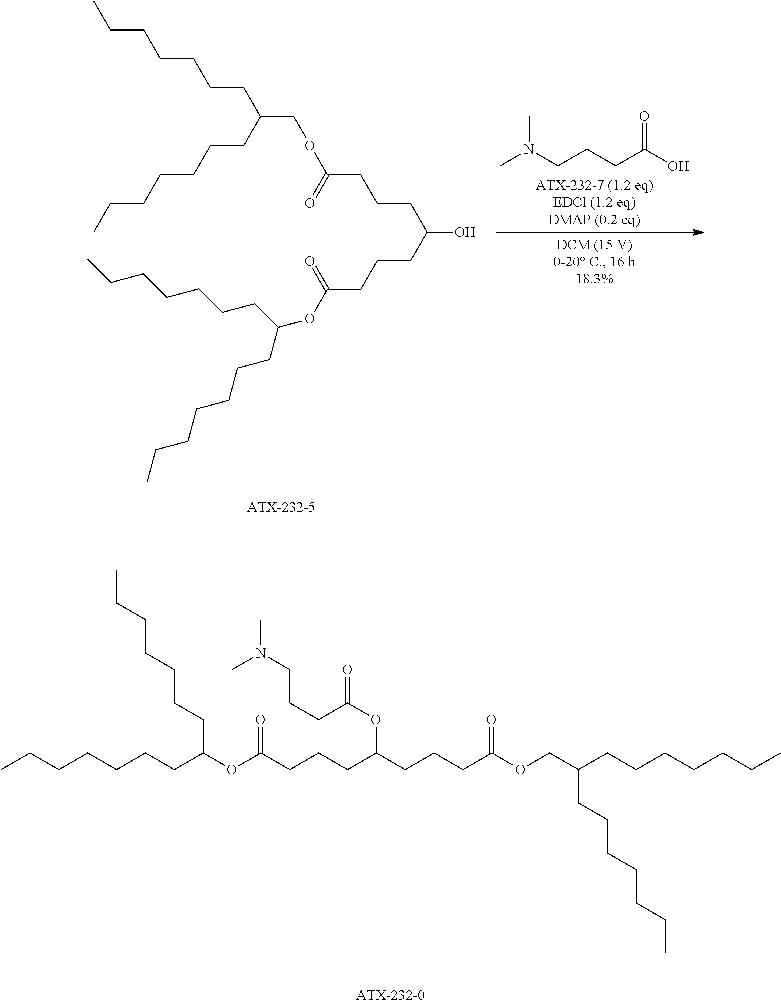

### Synthesis of ATX-232-4

Step 1:

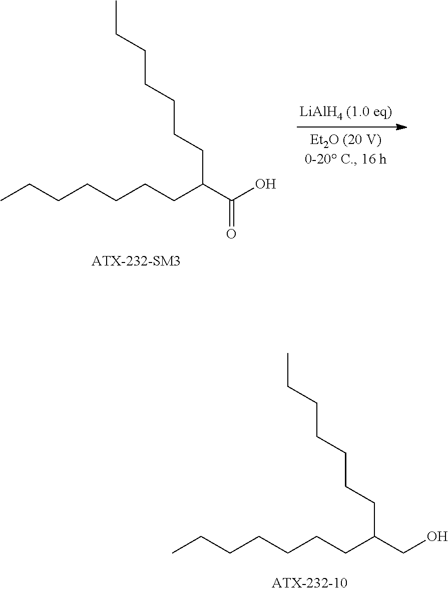 [SMILES: CCCCCCCC(CCCCCCC)C(=O)O ; CCCCCCCC(CO)CCCCCCC]

Into a 250 mL 3-necked round-bottom flask purged and maintained with an inert atmosphere of nitrogen, was placed ATX-232-SM3 (10.0 g, 1.0 equiv) in Et₂O (100 mL, 10 V) at room temperature. This was followed by LiAlH₄ (1.48 g, 1.0 equiv) at 0° C. The resulting solution was stirred for 16 h at room temperature. The reaction was then quenched by the addition of ice water (50 mL, 5 V). The resulting solution was extracted with EA (3*200 mL, 60 V) and the organic layers were combined. The organic layer was washed with brine (2*100 mL, 20 V) and dried with anhydrous Na₂SO₄, filtered and concentrated under vacuum. This resulted in 7.0 g (75% yield) ATX-232-10 as yellow oil. ¹H NMR (300 MHz, Chloroform-d) δ 4.18-4.11 (m, 1H), 3.57-3.55 (d, J=8 Hz, 2H), 1.44-1.28 (m, 25H), 0.93-0.85 (m, 6H).

Step 2:

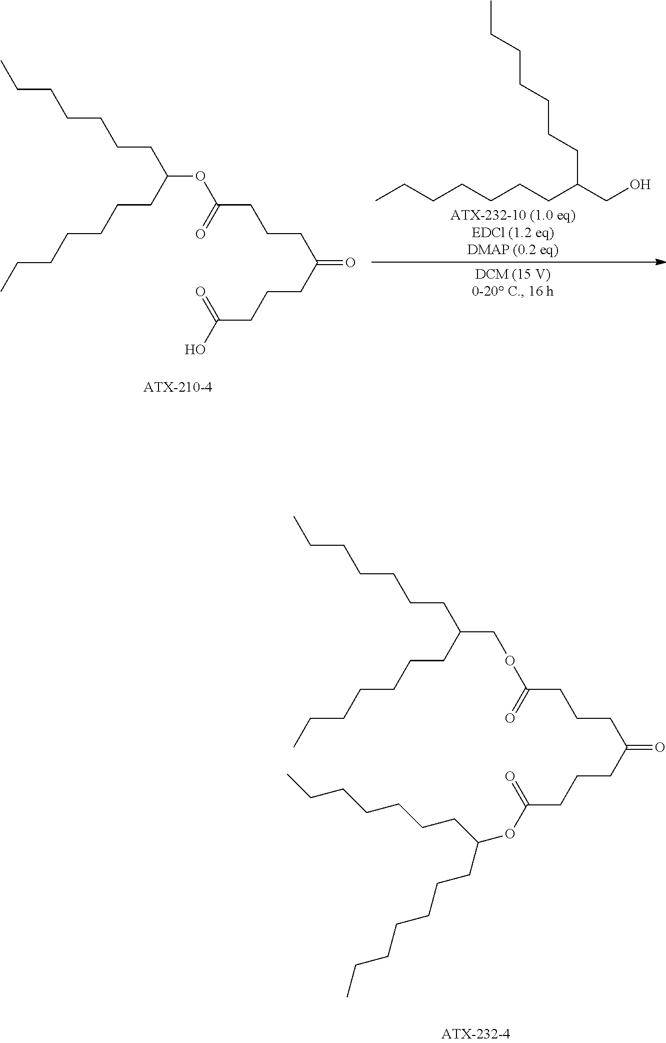 [SMILES: CCCCCCCC(CCCCCCC)OC(=O)CCCC(=O)CCCC(=O)O ; CCCCCCCC(CO)CCCCCCC ; CCCCCCCC(CCCCCCC)COC(=O)CCCC(=O)CCCC(=O)OC(CCCCCCC)CCCCCCC]

Into a 250 mL 3-necked round-bottom flask purged and maintained with an inert atmosphere of nitrogen, was placed ATX-210-4 (5.0 g, 1.0 equiv) and DCM (75 mL, 15 V) at room temperature. This was followed by the addition of ATX-232-10 (2.91 g, 1.0 equiv) and DMAP (0.3 g, 0.2 equiv) at room temperature, then EDCI (2.74 g, 1.2 equiv) was added at 0° C. The resulting solution was stirred for 16 h at room temperature. The reaction was then quenched by the addition of 1 mol/L aqueous HCl solution (25 mL, 5 V). The resulting solution was extracted with DCM (3*165 mL, 100 V) and the organic layers were combined. The organic layer was washed with brine (2*150 mL, 60 V) and dried with anhydrous Na₂SO₄, filtered and concentrated under vacuum. The crude mixture was adsorbed on 10 g of silica gel (type: ZCX-2, 100-200 mesh, 2.00 w./w.), and purified on a 100 g silica gel column, by eluting with DCM/MeOH gradient from 100:0 to 90:10. Fractions were pooled post TLC analysis. (DCM:MeOH=10:1) and concentrated under reduced pressure to get 5.5 g (72% yield) ATX-232-4 as yellow oil. ELSD A:water/0.05% TFA:B:CH₃CN/0.05% TFA 80:20 to 20:80 A/B at 3.5 min): RT 2.81 min, m/z (Calcd.) 636.57, (found) 659.55 (M+Na); H-NMR-PH-ARC-LIPID-230-2: (300 MHz, Chloroform-d): δ 4.89-4.85 (m, 1H), 3.99-3.97 (d, J=8 Hz, 2H), 2.50-2.46 (m, 4H), 2.36-2.29 (m, 4H), 1.95-1.85 (m, 4H), 1.52-1.51 (6H, m), 1.28 (48H, m), 0.91-0.84 (m, 12H).

### Synthesis of ATX-232-5

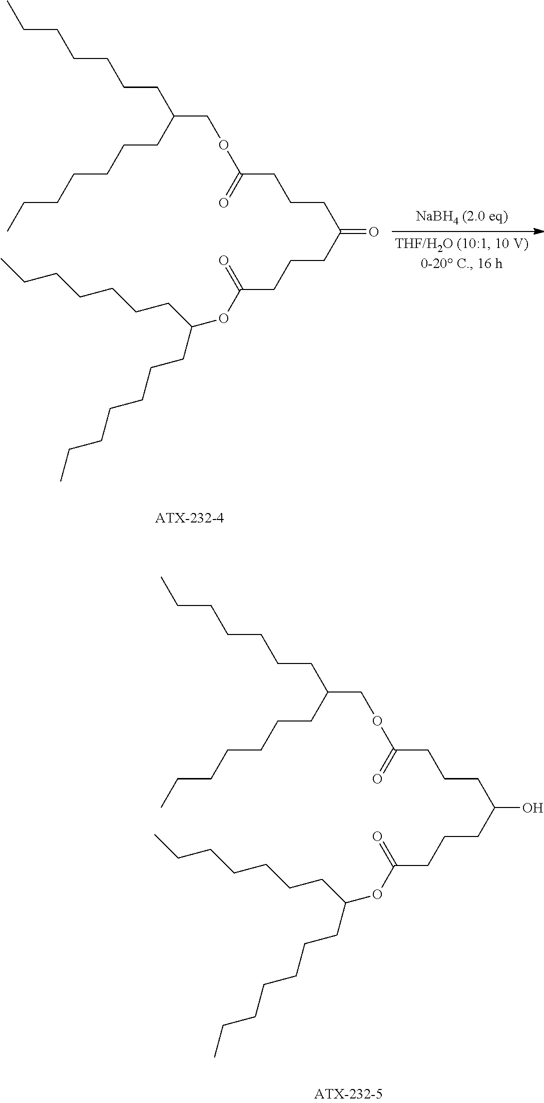 [SMILES: CCCCCCCC(CCCCCCC)COC(=O)CCCC(=O)CCCC(=O)OC(CCCCCCC)CCCCCCC ; CCCCCCCC(CCCCCCC)COC(=O)CCCC(O)CCCC(=O)OC(CCCCCCC)CCCCCCC]

Into a 100 mL 3-necked round-bottom flask purged and maintained with an inert atmosphere of nitrogen, was placed ATX-232-4 (5.5 g, 1.0 equiv) in THF/H₂O (10/1, 55 mL, 10 V) at room temperature. This was followed by the addition of NaBH₄ (0.88 g, 2.0 equiv) in several batches at 0° C. The resulting solution was stirred for 16 h at room temperature. The reaction was then quenched by the addition of ice water (27.5 mL, 5 V). The resulting solution was extracted with ethyl acetate (3*90 mL, 50 V) and the organic layers were combined. The organic layer was washed with brine (2*110 mL, 40 V), dried with anhydrous Na₂SO₄, filtered and concentrated under vacuum. The crude mixture was adsorbed on 11 g of silica gel (type: ZCX-2, 100-200 mesh, 2.00 w./w.), and purified on a 60 g silica gel column, by eluting with DCM/MeOH gradient from 100:0 to 90:10. Fractions were pooled post TLC analysis. (DCM:MeOH=10:1) and concentrated under reduced pressure to get 5.1 g (93% yield) ATX-232-5 as yellow oil. ELSD A:water/0.05% TFA:B:CH₃CN/0.05% TFA 80:20 to 20:80 A/B at 3.5 min): RT 2.81 min, m/z (Calcd.) 638.58, (found) 661.55 (M+Na); H-NMR-PH-ARC-LIPID-230-2: (300 MHz, Chloroform-d): δ 4.93-4.83 (m, 1H), 4.00-3.98 (d, J=8 Hz, 2H), 3.66-3.62 (m, 12H), 2.50-2.46 (m, 4H), 2.64-2.69 (m, 4H), 1.88-1.85 (m, 6H), 1.95-1.85 (m, 4H), 1.58-1.52 (m, 7H), 1.47-1.44 (m, 44H), 0.96-0.88 (m, 12H).

### Synthesis of ATX-232

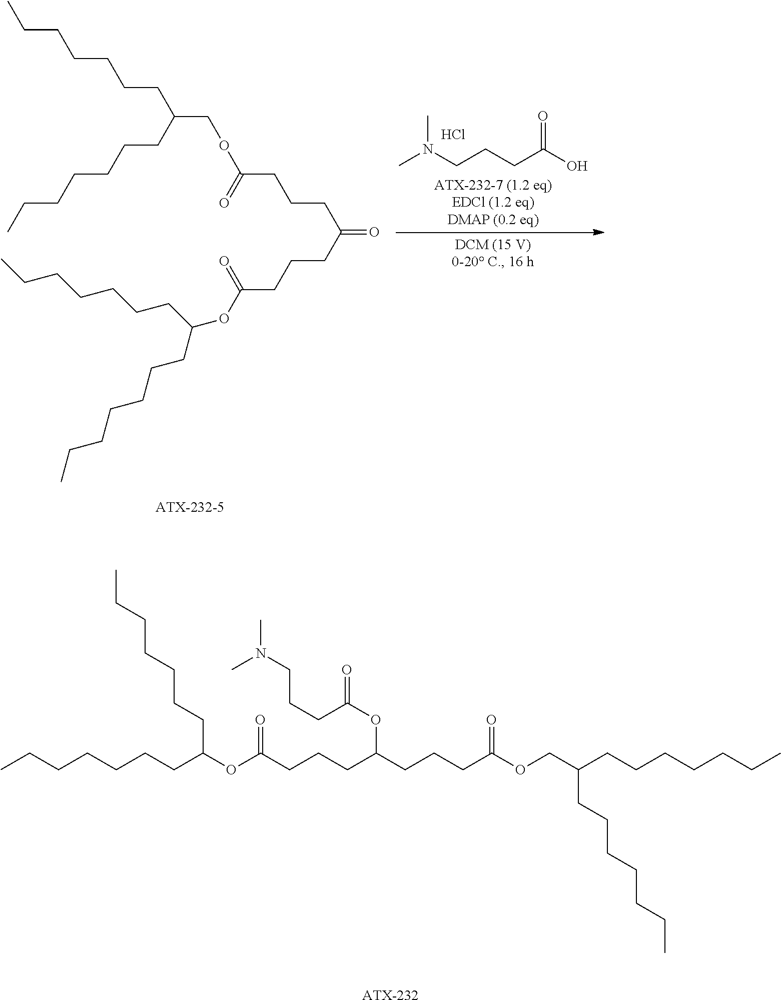

Into a 100 mL 3-necked round-bottom flask purged and maintained with an inert atmosphere of nitrogen, was placed ATX-232-5 (5.1 g, 1.0 equiv) in DCM (80 mL, 15 V) at room temperature. This was followed by the addition of ATX-232-7 (1.6 g, 1.2 equiv) and DMAP (0.21 g, 0.2 equiv) at room temperature, then EDCI (1.92 g, 1.2 equiv) was added at 0° C. The resulting solution was stirred for 16 h at room temperature. The reaction was then quenched by the addition of ice water (25 mL, 5 V). The resulting solution was extracted with DCM (3*80 mL, 50 V) and the organic layers were combined. The organic layer was washed with brine (2*100 mL, 40 V) and dried with anhydrous Na₂SO₄, filtered and concentrated under vacuum. The crude mixture was adsorbed on 11 g of silica gel (type: ZCX-2, 100-200 mesh, 2.00 w./w.), and purified on a 60 g silica gel column, by eluting with DCM/MeOH gradient from 100:0 to 90:10. Fractions were pooled post TLC analysis. (DCM:MeOH=10:1) and concentrated under reduced pressure to get 1.1 g (18.3% yield) ATX-232 as yellow oil. LC-MS-PH-ARC-ATX-232-0: (ES, m z): 752 [M+1]*; H-NMR-PH-ARC-ATX-232-0: (300 MHz, CDCl₃, ppm): δ 4.997-4.858 (m, 2H), 3.983 (d, J=5.7 Hz, 2H), 2.386-2.261 (m, 6H), 2.261 (s, 6H), 1.823 (quint, J=7.2 Hz, 2H), 1.799-1.512 (m, 13H), 1.289 (s, 46H), 0.922-0.882 (m, 12H).

### Example 10. Biological Data of the Compounds of the Present Invention

A variety of assays were conducted to assess the efficacy of lipids of the present disclosure. A description of these assays follows.

### Protocol for Factor VII Knock Down Evaluation

Lipid formulations comprising a FVII siRNA further described below were evaluated for their knockdown activity using the protocol of this example. In the FVII evaluation, seven to eight week-old, female Balb/C mice were purchased from Charles River Laboratories (Hollister, Calif.). The mice were held in a pathogen-free environment and all procedures involving the mice were performed in accordance with guidelines established by the Institutional Animal Care and Use Committee (IACUC). Lipid nanoparticles containing factor VII siRNA were administered intravenously at a dosing volume of 10 mL/kg and two dose levels (0.03 and 0.01 mg/kg). After 48 h, the mice were anesthetized with isoflurane and blood was collected retro-orbitally into Microtainer® tubes coated with 0.109 M sodium citrate buffer (BD Biosciences, San Diego, Calif.) and processed to plasma. Plasma specimens were tested for factor VII levels immediately or stored at −80° C. for later analysis. Measurement of FVII protein in plasma was determined using the colorimetric Biophen VII assay kit (Aniara Diagnostica, USA). Absorbance was measured at 405 nm and a calibration curve was generated using the serially diluted control plasma to determine levels of factor VII in plasma from treated animals, relative to the saline-treated control animals.

Protocol for hEPO mRNA Expression Evaluation
Lipid formulations comprising a hEPO mRNA below were evaluated for their ability to express hEPO in vivo according to the protocol of this example. All animal experiments were conducted using institutionally-approved protocols (IACUC). In this protocol, female Balb/c mice at least 6-8 weeks of age were purchased from Charles River Laboratory. The mice were intravenously injected with hEPO-LNPs via the tail vein with one of two dose levels of hEPO (0.1 and 0.03 mg/kg). After 6 hr, blood was collected with serum separation tubes, and the serum was isolated by centrifugation. Serum hEPO levels were then measured using an ELISA assay (Human Erythropoietin Quantikine IVD ELISA Kit, R&D Systems, Minneapolis, Md.).

### Mouse Plasma Stability

Lipid stock solution was prepared by dissolution of the lipid in isopropanol at the concentration of 5 mg/mL. A requisite volume of the lipid-isopropanol solution was then diluted to 100 μM concentration at a total volume of 1.0 mL with in 50:50 (v/v) ethanol/water. Ten microliters of this 100 μM solution was spiked into 1.0 mL of mouse plasma (BioIVT, Cat. No.: MSE00PLNHUNN, CD-1 mouse, anticoagulant: sodium heparin, not filtered) that was pre-warmed to 37° C. and and was stirred at 50 rpm with a magnetic stir bar. The starting concentration of lipids in plasma was thus 1 μM. At time points 0, 15, 30, 45, 60 and 120 min, 0.1 mL of the plasma was withdrawn from the reaction mixture and the protein was precipitated by adding 0.9 mL of ice-cold 4:1 (v/v) acetonitrile/methanol with 1 μg/mL of a selected internal standard lipid added. After filtration through a 0.45 micron 96-well filtering plate, the filtrates were analyzed by LC-MS (Thermo Fisher's Vanquish UHPLC-LTQ XL linear ion trap Mass Spectrometer); Waters XBridge BEH Shield RP18 2.5 □m (2.1×100 mm) column with its matching guard column. Mobile phase A was 0.1% formic acid in water, and mobile phase B was 0.1% formic acid in 1:1 (v/v) acetonitrile/methanol. Flow rate was 0.5 min/min. Elution gradient was: Time 0-1 min: 10% B; 1-6 min: 10%-95% B; 6-8.5 min: 95% B; 8.5-9 min: 95%-10% B; 9-10 min: 10% B. Mass spectrometry was in positive scanning mode from 600-1100 m/z. The peak of the molecular ion of the lipids was integrated in the extracted ion chromatography

(XIC) using Xcalibur software (Thermo Fisher). The relative peak area compared to T=0, after normalization by the peak area of the internal standard, was used as the percentage of the lipid remaining at each time point. T_{1/2} values were calculated using the first-order decay model.

### In Vivo Biodegradability Assay

In vivo biodegradability assay was performed to assess the biodegradability of lipids in the LNP. Briefly, mice were injected with either 0.1 or 0.03 mg/Kg dose and after 24 or 48 hours mice livers were collected. To measure the concentration of lipids in the mouse liver, liver samples were homogenized in appropriate buffer in 1-10 dilution and mixed with the same amount of stabilized plasma. The samples were then mixed with organic solvents spiked with internal standard to precipitate proteins. After centrifugation, supernatant was diluted further with organic solvent before sample analysis by LC-MS. In LC-MS analysis, positive electrospray ionization was used, and multiple reaction monitoring (MRM) parameters were set up to specifically target the lipid analyte and internal standard. Calibration standards were prepared in stabilized plasma and mixed with same amount of homogenization buffer before protein precipitation. Quality control samples with known amounts of lipid was prepared in blank liver homogenate to monitor the precision and accuracy of the assay.

TABLE 1
Biological Assays Data

| | Compound- | ATX- | ATX- | ATX- | ATX- | ATX- | ATX- | ATX- | ATX- |
|---|---|---|---|---|---|---|---|---|---|
| ATX#/Attributes | 10111 | 193 | 200 | 201 | 202 | 209 | 210 | 231 | 232 |
| FVII KD % _ | 95 | 100 | 97.5 | 97.5 | 83.22 | 90.6 | 97.65 | 86 | 90 |
| 0.03 mpk | | | | | | | | | |
| FVII KD % _ | 89 | 95.6 | 87.1 | 87.4 | 61.42 | 65.7 | 87 | 72 | 74 |
| 0.01 mpk | | | | | | | | | |
| EPO expression | 105 | 264 | 494 | 523 | 447 | 65 | 329 | 1706 | 2115 |
| (ng/mL)_0.03 | | | | | | | | | |
| mpk | | | | | | | | | |
| EPO expression | 213 | 498 | 828 | 680 | 531 | 290 | 662 | 5446 | 510 |
| (ng/mL)_0.1 | | | | | | | | | |
| mpk | | | | | | | | | |

TABLE 2
Half-Life and Degradability Data

| ATX#/ | Compound- | ATX- | ATX- |
|---|---|---|---|
| Attributes | 10111 | 209 | 231 |
| Plasma half life | 91 | 80 | 89 |
| % remaining | | | |
| after 2 h | | | |
| In vivo | 566 | 0 | 0 |
| degradability | | | |
| ng/g of tissue at | | | |
| 48 h_0.03 mpk | | | |
| In vivo | 2613 | 647 | 0 |
| degradability | | | |
| ng/tissue at | | | |
| 48 h_0.1 mpk | | | |

Compound-10111 is shown below, and listed in page 243 of WO 2021/030701:

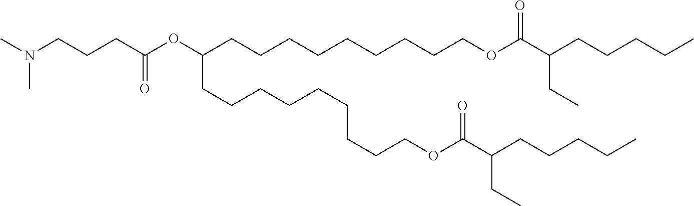 [SMILES: CCCCCC(CC)C(=O)OCCCCCCCCCC(CCCCCCCCCOC(=O)C(CC)CCCCC)OC(=O)CCCN(C)C]

Table 3 below shows the calculated Log D (c Log D) and calculated pKa (cpKa), as well as measured pKa in parenthesis values of the ATX compounds. The c Log D and cpKa values are generated by ACD Labs Structure Designer v12.0.

TABLE 3
cLogD and cpKa values

| | | c-pKa |
|---|---|---|
| ATX-# | cLogD | (pKa) |
| 193 | 13.74 | 9.36 |
| | | (6.35) |
| 200 | 14.01 | 9.37 |
| | | (6.78) |
| 201 | 13.23 | 9.37 |
| | | (6.74) |
| 202 | 14.42 | 9.31 |
| | | (6.17) |
| 209 | 13.51 | 9.37 |
| 210 | 11.70 | 9.35 |
| | | (6.53) |
| 230 | 12.56 | 9.34 |
| 231 | 14.02 | 9.35 |
| 232 | 14.25 | 9.36 |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula I, or a pharmaceutically acceptable salt thereof: 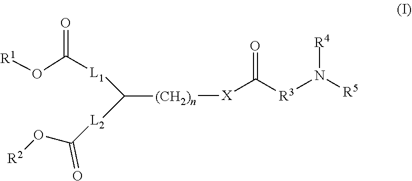
wherein:
R¹ and R² are each independently (CH₃(CH₂)ₘ)₂CH—, (CH₃(CH₂)ₘ)(CH₃(CH₂)ₘ₋₁)CH, (CH₃(CH₂)ₘ)(CH₃(CH₂)ₘ₋₂)CH, (CH₃(CH₂)ₘ)₂CHCH₂—, or (CH₃(CH₂)ₘ)(CH₃(CH₂)ₘ₋₁)CHCH₂—, wherein m is 4-11;
L¹ and L² are each independently absent, a linear C₁₋₅ alkylene, or (CH₂)ₚ—O—(CH₂)_{q}, wherein p and q are each independently 1-3;
R³ is a linear C₂₋₅ alkylene optionally substituted with one or two methyl groups;
R⁴ and R⁵ are each independently H or C₁₋₆ alkyl;
X is O or S; and
n is 0-2.

2. The compound of claim 1, wherein R¹ and R² are each independently selected from (CH₃(CH₂)ₘ)₂CH—, and (CH₃(CH₂)ₘ)₂CHCH₂—.

3.-4. (canceled)

5. The compound of claim 1, wherein R¹ and R² are each independently selected from (CH₃(CH₂)ₘ)(CH₃(CH₂)ₘ₋₁)CH, (CH₃(CH₂)ₘ)(CH₃(CH₂)ₘ₋₂)CH, and (CH₃(CH₂)ₘ)(CH₃(CH₂)ₘ₋₁)CHCH₂—.

6. (canceled)

7. The compound of claim 1, wherein m is 4 to 8.

8.-11. (canceled)

12. The compound of claim 1, wherein L¹ and L² are each independently C₂₋₅ alkylene or (CH₂)ₚ—O—(CH₂)_{q}.

13-14. (canceled)

15. The compound of claim 1, wherein L¹ and L² are each absent.

16.-19. (canceled)

20. The compound of claim 1, wherein n is 0-1.

21.-22. (canceled)

23. The compound of claim 1, selected from the group consisting of: 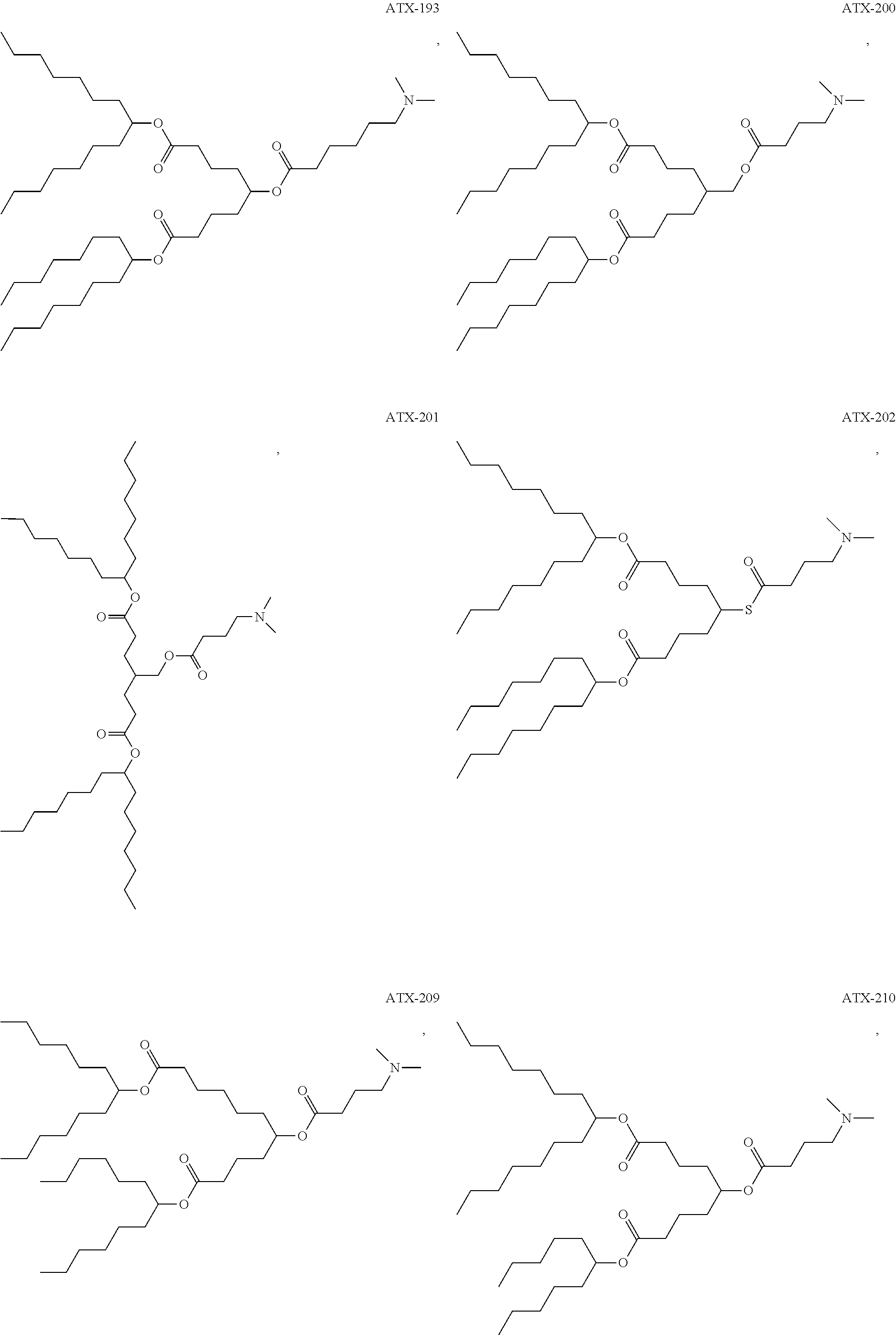 [SMILES: CCCCCCCC(CCCCCCC)OC(=O)CCCC(CCCC(=O)OC(CCCCCCC)CCCCCCC)OC(=O)CCCCCN(C)C ; CCCCCCCC(CCCCCCC)OC(=O)CCCC(CCCC(=O)OC(CCCCCCC)CCCCCCC)COC(=O)CCCN(C)C ; CCCCCCCC(CCCCCCC)OC(=O)CCC(CCC(=O)OC(CCCCCCC)CCCCCCC)COC(=O)CCCN(C)C ; CCCCCCCC(CCCCCCC)OC(=O)CCCC(CCCC(=O)OC(CCCCCCC)CCCCCCC)SC(=O)CCCN(C)C ; CCCCCCC(CCCCCC)OC(=O)CCCCCC(CCCC(=O)OC(CCCCCC)CCCCCC)OC(=O)CCCN(C)C ; CCCCCCCC(CCCCCCC)OC(=O)CCCC(CCCC(=O)OC(CCCCC)CCCCC)OC(=O)CCCN(C)C] 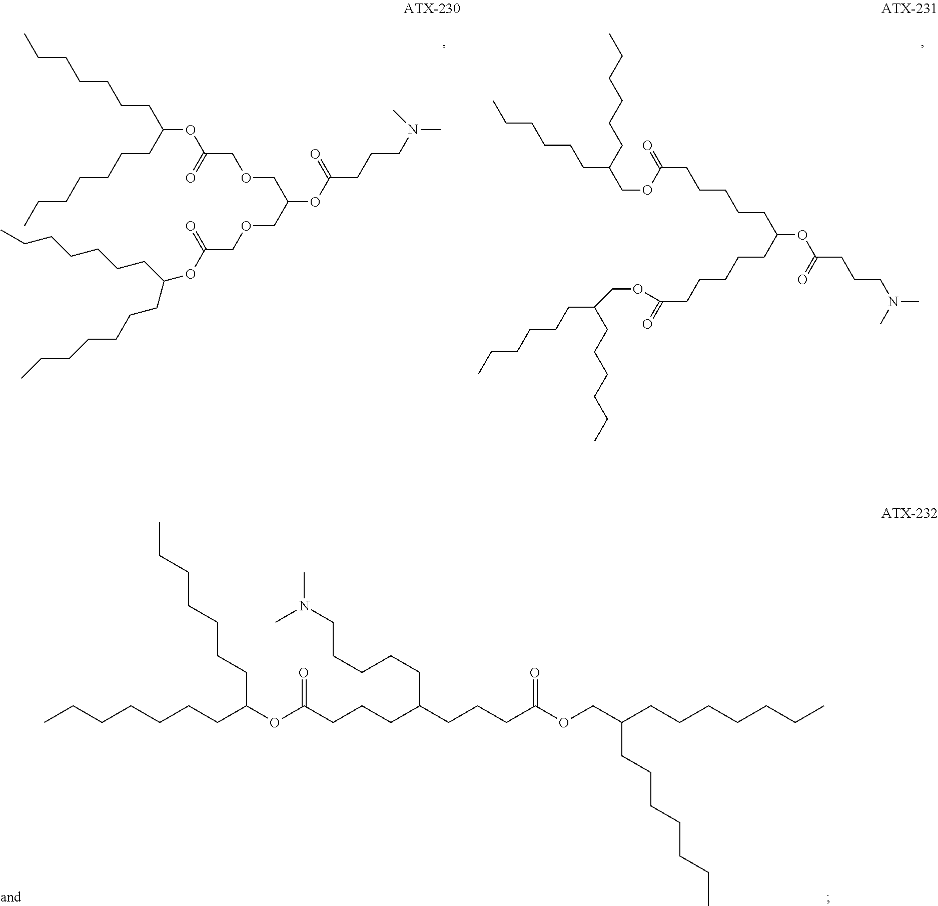 [SMILES: CCCCCCCC(CCCCCCC)OC(=O)COCC(COCC(=O)OC(CCCCCCC)CCCCCCC)OC(=O)CCCN(C)C ; CCCCCCC(CCCCCC)COC(=O)CCCCCC(CCCCCC(=O)OCC(CCCCCC)CCCCCC)OC(=O)CCCN(C)C ; CCCCCCCC(CCCCCCC)COC(=O)CCCC(CCCCCN(C)C)CCCC(=O)OC(CCCCCCC)CCCCCCC]
or a pharmaceutically acceptable salt thereof.

24. The compound of claim 23, wherein the compound is ATX-193:

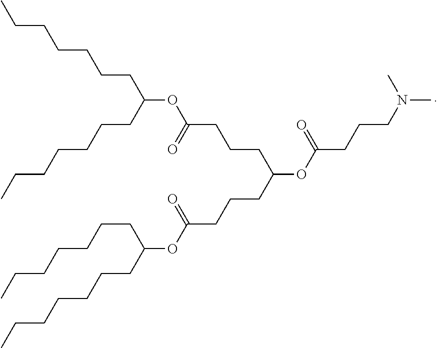 [SMILES: CCCCCCCC(CCCCCCC)OC(=O)CCCC(CCCC(=O)OC(CCCCCCC)CCCCCCC)OC(=O)CCCN(C)C]

25. The compound of claim 23, wherein the compound is ATX-200:

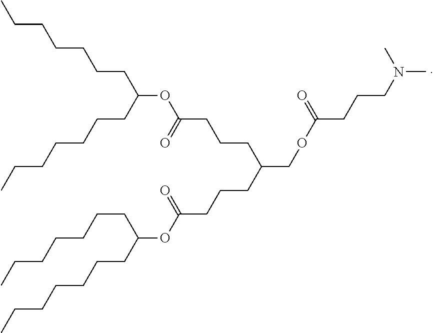 [SMILES: CCCCCCCC(CCCCCCC)OC(=O)CCCC(CCCC(=O)OC(CCCCCCC)CCCCCCC)COC(=O)CCCN(C)C]

26. The compound of claim 23, wherein the compound is ATX-201:

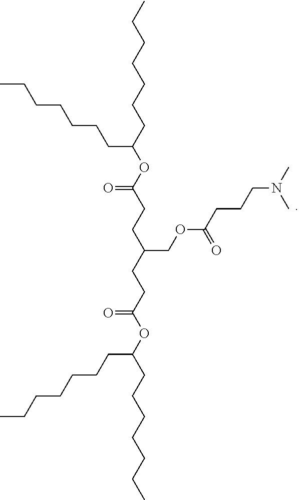 [SMILES: CCCCCCCC(CCCCCCC)OC(=O)CCC(CCC(=O)OC(CCCCCCC)CCCCCCC)COC(=O)CCCN(C)C]

27. The compound of claim 23, wherein the compound is ATX-202:

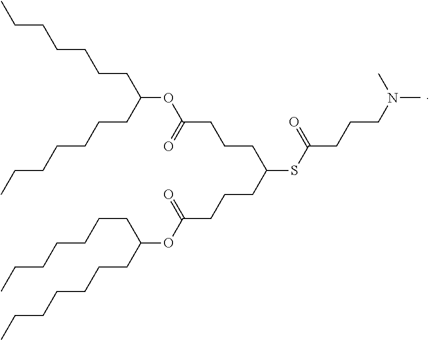 [SMILES: CCCCCCCC(CCCCCCC)OC(=O)CCCC(CCCC(=O)OC(CCCCCCC)CCCCCCC)SC(=O)CCCN(C)C]

28. The compound of claim 23, wherein the compound is ATX-209:

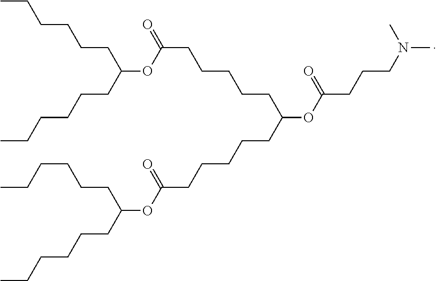 [SMILES: CCCCCCC(CCCCCC)OC(=O)CCCCCC(CCCCCC(=O)OC(CCCCCC)CCCCCC)OC(=O)CCCN(C)C]

29. The compound of claim 23, wherein the compound is ATX-210:

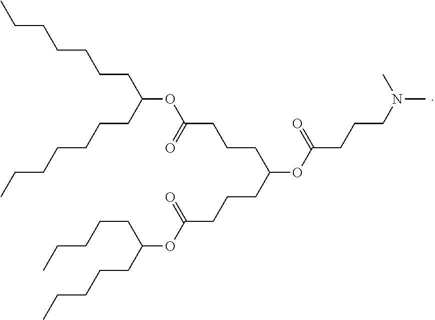 [SMILES: CCCCCCCC(CCCCCCC)OC(=O)CCCC(CCCC(=O)OC(CCCCC)CCCCC)OC(=O)CCCN(C)C]

30. The compound of claim 23, wherein the compound is ATX-230:

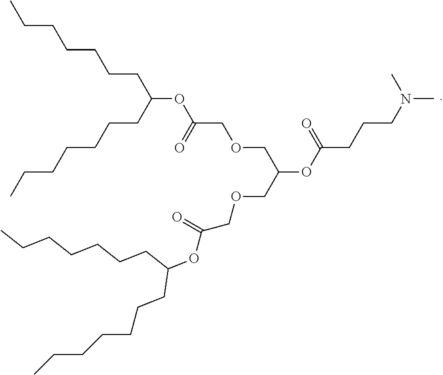 [SMILES: CCCCCCCC(CCCCCCC)OC(=O)COCC(COCC(=O)OC(CCCCCCC)CCCCCCC)OC(=O)CCCN(C)C]

31. The compound of claim 23, wherein the compound is ATX-231:

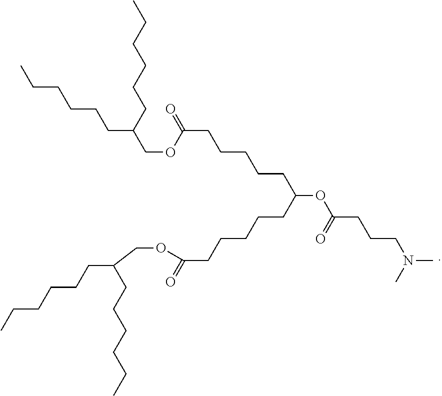 [SMILES: CCCCCCC(CCCCCC)COC(=O)CCCCCC(CCCCCC(=O)OCC(CCCCCC)CCCCCC)OC(=O)CCCN(C)C]

32. The compound of claim 23, wherein the compound is ATX-232:

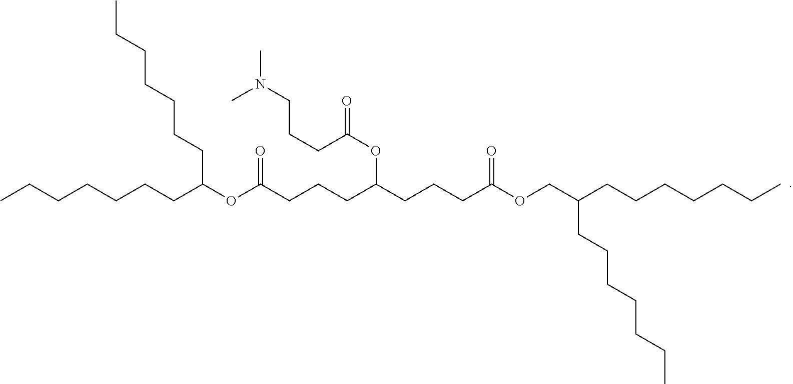 [SMILES: CCCCCCCC(CCCCCCC)COC(=O)CCCC(CCCC(=O)OC(CCCCCCC)CCCCCCC)OC(=O)CCCN(C)C]

33. A lipid composition comprising a nucleic acid and a compound of claim 1.

34. The lipid composition of claim 33, wherein the nucleic acid is selected from an siRNA, an mRNA, a self-replicating RNA, a DNA plasmid, and an antisense oligonucleotide.

35. The lipid composition of claim 33, wherein the nucleic acid is a mRNA or a self-replicating RNA comprising a coding region that encodes a therapeutic protein of interest.

36. The lipid composition of claim 35, wherein the therapeutic protein of interest is an enzyme, and antibody, an antigen, a receptor, or a transporter.

37. The lipid composition of claim 35, wherein the therapeutic protein of interest is a gene-editing enzyme.

38. The lipid composition of claim 37, wherein the gene-editing enzyme is selected from a TALEN, a CRISPR, a meganuclease, or a zinc finger nuclease.

39.-40. (canceled)

41. The lipid composition of claim 33, comprising a lipid nanoparticle encapsulating the nucleic acid, wherein the average particle size of the lipid nanoparticle is less than about 100 nm.

42.-48. (canceled)

49. The lipid composition of claim 41 wherein the lipid nanoparticle further comprises a helper lipid selected from: dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), and phosphatidylcholine (PC).

50. (canceled)

51. The lipid composition of claim 41 further comprising cholesterol.

52. The lipid composition of claim 41 further comprising a polyethylene glycol(PEG)-lipid conjugate.

53.-54. (canceled)

55. The lipid composition of claim 41, wherein the lipid nanoparticle comprises about 45 mol % to 65 mol % of claim 1 about 2 mol % to about 15 mol % of a helper lipid, about 20 mol % to about 42 mol % of cholesterol, and about 0.5 mol % to about 3 mol % of a PEG-lipid conjugate.

56.-57. (canceled)

58. The lipid composition of claim 41, wherein the lipid nanoparticle has a total lipid:nucleic acid weight ratio of about 50:1 to about 10:1.

59.-70. (canceled)

71. A method of treating a disease in a subject in need thereof, comprising administering a therapeutically effective amount to the subject, the lipid composition of claim 41.

72. (canceled)

73. A method of expressing a protein or polypeptide in a target cell, comprising contacting the target cell with a lipid composition of claim 41.

74. (canceled)

75. (canceled)
